(19) United States
(12) Patent Application Publication
(10) Pub. No.: US 2017/0007610 A1
(43) Pub. Date: Jan. 12, 2017

(54) NOVEL HETEROBICYCLIC COMPOUNDS AS KAPPA OPIOID AGONISTS

(71) Applicant: CADILA HEALTHCARE LIMITED, Ahmedabad, Gujarat (IN)

(72) Inventors: Ranjit C. DESAI, Ahmedabad, Gujarat (IN); Rajesh BAHEKAR, Ahmedabad, Gujarat (IN); Vijay PRAJAPATI, Ahmedabad, Gujarat (IN); Pankaj R. PATEL, Ahmedabad, Gujarat (IN)

(21) Appl. No.: 15/113,497

(22) PCT Filed: Jan. 23, 2015
(86) PCT No.: PCT/IN2015/000043

§ 371 Date: Jul. 22, 2016

(30) Foreign Application Priority Data
Jan. 24, 2014 (IN) 272/MUM/2014

(51) Int. Cl. A61K 31/517; C07D 401/12; A61K 31/4709; A61K 31/4725; C07D 405/12; C07D 401/14; C07D 403/12; A61K 31/498; A61K 31/404; C07D 513/06; A61K 31/437; A61K 45/06; A61K 31/4025

(52) U.S. Cl. CPC A61K 31/517; A61K 45/06; C07D 401/12; A61K 31/4709; A61K 31/4725; C07D 405/12; A61K 31/4025; C07D 403/12; A61K 31/498; A61K 31/404; C07D 513/06; A61K 31/437; C07D 401/14

(57) ABSTRACT

The present invention relates to novel compounds of the general formula (I), which are selective and peripherally acting KOR agonist, their tautomeric forms, their enantiomers, their diastereoisomers, their stereoisomers, their pharmaceutically accepted salts, or prodrugs thereof which are useful in the treatment or prevention of diseases in which the Kappa (κ) opioid receptors (KOR) are involved, such as treatment or prevention of visceral pain, hyperalgesia, rheumatoid arthritic inflammation, osteoarthritic inflammation, IBD inflammation, IBS inflammation, ocular inflammation, otitic inflammation or autoimmune inflammation. The invention also relates to process for the manufacture of said compounds, and pharmaceutical compositions containing them and their use.

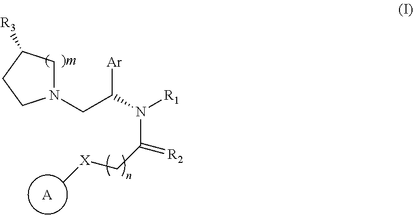

### FIELD OF INVENTION

The present invention relates to novel compounds of the general formula (I), which are selective and peripherally acting KOR agonist, their tautomeric forms, their enantiomers, their diastereoisomers, their stereoisomers, their pharmaceutically accepted salts, or prodrugs thereof which are useful in the treatment or prevention of diseases in which the Kappa (κ) opioid receptors (KOR) are involved, such as treatment or prevention of visceral pain, hyperalgesia, rheumatoid arthritic inflammation, osteoarthritic inflammation, IBD inflammation, IBS inflammation, ocular inflammation, otitic inflammation or autoimmune inflammation. The invention also relates to process for the manufacture of said compounds, and pharmaceutical compositions containing them and their use.

### BACKGROUND OF THE INVENTION

There are three types of opioid receptors Mu (μ), Kappa (κ), and Delta (δ)), found to be expressed in both the CNS and in the periphery and the available opioid analgesics mediate their effects through these opioid receptors (Evans, C., Keith, J. D., Morrison, H., Magendzo, K and Edwards, R., Science, 258, 1952-1955, 1992; Cox, B. M., Mol. Pharmacol., 83, 723-728, 2013; Chen, Y., Mestek, A., Liu, J., Hurley, J and Yu, L., Mol. Pharmacol., 44, 8-12, 1993; Meng, F., Xie, G. X., Thompson, R., Mansour, A., Goldstein, A., Watson, S. J and Akil, H., Proc. Natl. Acad. Sci., U.S.A., 90, 9954-9958, 1993; Simonin, F., Gaveriaux, R. C., Befort, K., Matthes, H., Lannes, B., Micheletti, G., Maffei, M. G., Charron, G., Bloch, B and Kieffer, B., Proc. Natl. Acad. Sci., U.S.A., 92, 7006-7010, 1995; Stein, C., Anesth. Analg., 76, 182-191, 1993). Most of the opioid analgesics at present, for example, morphine, act by binding to the μ-opioid receptor, and their analgesic potency are associated with a spectrum of undesirable side effects, such as physical dependence, respiratory depression, urinary retention, constipation, euphoria/dysphoria and constipation (Pasternak, G. W., Clin. Neuropharmacol., 16, 1-18, 1993).

In recent years, considerable attention has been focused on the development of receptor selective κ-agonists as potent and efficacious analgesics devoid of the undesirable side effects of the μ analgesics (Barber, A and Gottschlich, R., Med. Res. Rev., 12, 525-562, 1992). Unlike agonist at δ and μ receptors, agonist at κ-opioid receptors do not elicit constipation and euphoria. The κ-opioid receptors are members of the superfamily of G protein-coupled receptors (GPCRs). Agonist binding to the receptors activates the intracellular associated Gi protein, which decreases Ca²⁺ channel conductance or inhibits adenylyl cyclase (AC) (Prather, P. L., McGinn, T. M., Claude, P. A., Liu-Chen, L. Y., Loh, H. H and Law, P. Y., Mol. Brain. Res., 29, 336-346, 1995). Hence, κ-opioid agonists have been suggested to have potential for treatment of incisional/inflammatory pain, burn injury pain (Field, M. J., Carnell, A. J., Gonzalez, M. I., McCleary, S., Oles, R. J., Smith, R., Hughes, J and Singh, L., Pain, 80, 383-389, 1999), neuropathic pain (Catheline, G., Guilbaud, G and Kayser, V., Eur. J. Pharmacol., 357, 171-178, 1998), visceral pain including dysmenorrhea or gastrointestinal pain (DelgadoAros, S., Chial, H. J., Camilleri, M., Szarka, L. A., Weber, F. T., Jacob, J., Ferber, I., McKinzie, S., Burton, D. D and Zinsmeister, A. R., Am. J. Physiol. Gastrointest. Liver Phsyiol., 284, G558-G566, 2002), Irritable bowel syndrome (IBS) (Dapoigny, M., Abitbol, J. L., Fraitag, B., Digest. Dis. Sci., 40, 2244-2249, 1995; Mangel, A. W., Bornstein, J. D., Hamm, L. R., Buda, J., Wang, J., Irish, W., Urso, D., Pharmacol. Ther., 28, 239-249, 2008) rheumatoid arthritis (Endoh, T., Tajima, A., Suzuki, T., Kamei, J., Suzuki, T., Narita, M., Tseng, L and Nagase, H., Eur. J. Pharmacol. 387, 133-140, 2000) and anti-pruritis effects (Peters, G and Gaylor, S., Clin. Pharmacol. Ther., 51, PPF-5, 1989). Walker et al., (Walker, J. S., Adv. Exp. Med. Biol., 521, 148-60, 2003) appraised the anti-inflammatory properties of kappa agonists for treatment of osteoarthritis, rheumatoid arthritis, inflammatory bowel disease and eczema.

Bileviciute-Ljungar et al., (Bileviciute-Ljungar, T. Saxne, and M. Spetea, Rheumatology, 45, 295-302, 2006) describe the reduction of pain and degeneration in Freund's adjuvant-induced arthritis by the kappa agonist U-50,488. Thus, the κ-receptors represent important therapeutic targets (Pan, Z. Z., Tershner, S. A., Fields, H. L., Nature, 389, 382-385, 1997; Chavkin, C., Neuropsychopharmacology, 36, 369-370, 2011).

κ-opioid receptors exist extensively in the central nervous system (CNS) and play important roles in many physiological and pathological functions. Inspite of such potential applications, clinical studies have shown that κ-receptor agonist elicit severe centrally mediated side effects generally described as “dysphoric actions” (Pfeiffer, A., Brantl, V., Herz, A and Emrich, H. M., Science, 233, 774-776, 1986), water diuresis (Dykstra, L. A., Gmerek, D. E., Winger, G and Woods, J. H., J. Pharmacol. Exp. Ther., 242, 413-420, 1987) and psychotomimetic effects (Rimoy, G. H., Wright, D. M., Bhaskar, N. K., Rubin, P. C, Eur. J. Clin. Pharmacol. 46 (3), 203-207, 1994). These side effects have apparently halted further clinical development for this class of compounds. Many studies have shown that opiates have peripheral analgesic effects, especially under inflammatory or hyperalgesic conditions (Barber, A and Gottschlich, R., Med. Res. Rev., 12, 525-562, 1992).

Agonist at κ-opioid receptors have been shown to produce analgesia and decrease inflammation in models of rheumatoid arthritis after local administration (Wilson, J. L., Nayanar, V and Walker, J. S., Br. J. Pharmacol., 118, 1754-1760, 1996). Restricted CNS penetration is a common strategy to reduce central side effects of drugs with beneficial peripheral actions. Attempts were made to develop peripherally restricted κ-opioid agonists, such as ICI204448 (Shaw, J. S., Carroll, J. A., Alcoc, P and Main, B. G., Br. J. Pharmacol., 96, 986-992, 1989), GR94839 (Rogers, H., Birch, P. J., Harrison, S. M., Palmer, E., Manchee, G. R., Judd, D. B., Naylor, A., Scopes, D. I. C and Hayes, A. G., Br. J. Pharmacol., 106, 783-789, 1992) and EMD61753/Asimadoline (Barber, A., Bartoszyk, G. D., Bender, H. M., Gottschlich, R., Greiner, H. E., Harting, J., Mauler, F., Minck, K. O., Murray, R. D., Simon, M and Seyfried, C. A., Br. J. Pharmacol., 113, 1317-1327, 1994).

Unfortunately, other than Asimadoline, most of these compounds were discontinued in clinical trials due to either poor bioavailability, lack of efficacy or CNS side effects at analgesic doses (Barber, A and Gottschlich, R., Exp. Opin. Invest. Drugs, 6, 1351-1368, 1997). Asimadoline was designed and synthesized to differentiate itself from other reported peripheral KOR agonists such as ICI 204448, GR94839, and BRL 52974. Asimadoline is an amphiphilic molecule that contains a hydrophobic diphenyl methyl group and a hydrophilic hydroxyl group. Asimadoline successfully passed a phase II clinical trial in irritable bowel syndrome (IBS) and currently, it is under phase III clinical trial for the treatment of patients with diarrhea-predominant IBS (D-IBS). CR665 and CR845 are tetrapeptides consisting of all D-amino acids that bind very potently and selectively to KOR. Dooley et al., (Dooley, C. T., Ny, P., Bidlack, J. M and Houghten, R. A., J. Biol. Chem., 273, 18848-18856, 1998) reported the discovery of tetrapeptide (FE200041/CR665) as a high affinity and selective κ-opioid agonist. The data demonstrate that FE200041 is a highly selective κ-opioid antinociceptive agent without CNS side effects at doses higher than efficacy doses. The peripheral antinociceptive actions of FE20041 suggest that it is possible to develop peripherally restricted opioid peptides for use in controlling pain. Similarly, in Phase I study, CR845 appeared to be well tolerated with no signs of dysphoria or psychotomimetic effects and provides the opportunity to see the potential analgesic activity of a peripheral KOR agonist which to date has been shown to be devoid of serious CNS adverse events.

### PRIOR ART

The most important selective κ-agonists developed so far are the arylacetamide derivatives. Since the discovery of the one of the first selective arylacetamide κ-agonists (U-50,488), in the early 1970s, which displayed analgesic effects invivo and did not produce respiratory depression, constipation, or tolerance, a number of related, but chemically diverse, arylacetamide κ-agonists have been reported (Lahti, R. A., VonVoigtlander, P. F., Barsuhn, C., Life Sci., 31, 2257-2260, 1982). Among them, ICI 199441, were found to be 146-fold more potent than U-50,488 invitro. However, these centrally acting κ-agonists produced their own set of CNS side effects such as dysphoria and diuresis, which prevented their further development as analgesic therapeutics. There has been an interest in the preparation of peripherally acting opioid agonists that have limited or no access to the CNS in an effort to reduce or eliminate these side effects (Stein, C., Anesth. Analg., 76, 182-191, 1993; Stein, C and Lang, L. J., Curr. Opin. Pharmacol., 9, 3-8, 2009).

Introducing polar or charged group into ligands has been attempted in order to enhance their CNS/PNS (peripheral nervous system) selectivity. However, polarization of the opioid may result in significant reduction in potency. Thus a continuing need exists for selective and potent opioid ligands with high κ-receptor activity and low CNS penetration (DeHaven-Hudkins, D. L and Dolle, R. E., Curr. Pharm. Des., 10, 743-757, 2004). Various classes of compounds featuring KOR agonist activity have been described in the literature.

U.S. Pat. No. 5,688,955 discloses substituted piperidines, substituted naphthalenes, aryl-substituted amides and cyclohexyl-substituted amides of the following general formula having κ opioid agonist activity (US, 1997, 5688955).

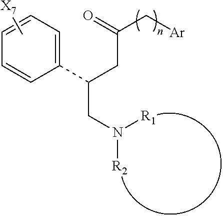

U.S. Pat. No. 5,804,595 discloses amino acid conjugates of substituted 2-phenyl-N-[1-(phenyl)-2-(1-heterocycloalkyl-or heterocycloaryl-)ethyl]acetamides allegedly useful for selectively agonizing κ opioid receptors in mammalian tissue (US, 1998, 5804595).

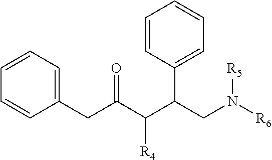

U.S. Pat. No. 6,133,307 discloses κ opioid agonists which are useful in the treatment of arthritis, hypertension, pain, inflammation, migraine, inflammatory disorders of the gastrointestinal tract, IBS and psoriasis (US, 2000, 6133307).

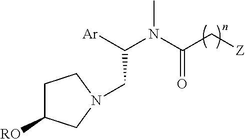

U.S. Pat. No. 7,160,902 discloses amide derivatives which are useful for treating and/or preventing gastrointestinal disorders, pain and pruritus (US, 2007, 7160902).

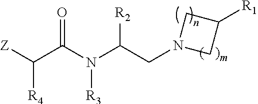

We herein disclose series of novel compounds of the general formula (I), which are selective and peripheral KOR agonist, useful for the treatment or prevention of diseases in which the Kappa (κ) opioid receptors (KOR) are involved, such as treatment or prevention of visceral pain, hyperalgesia, rheumatoid arthritic inflammation, osteoarthritic inflammation, IBD inflammation, IBS inflammation, ocular inflammation, otitic inflammation or autoimmune inflammation.

### SUMMARY OF THE INVENTION

The present invention relates to novel compounds of the general formula (I) their tautomeric forms, their enantiomers, their diastereoisomers, their stereoisomers, their pharmaceutically accepted salts, which are useful in the treatment or prevention of diseases in which the Kappa (κ) opioid receptors (KOR) are involved, such as treatment or prevention of visceral pain, hyperalgesia, rheumatoid arthritic inflammation, osteoarthritic inflammation, IBD inflammation, IBS inflammation, ocular inflammation, otitic inflammation or autoimmune inflammation. The invention also relates to process for the manufacture of said compounds, and pharmaceutical compositions containing them and their use.

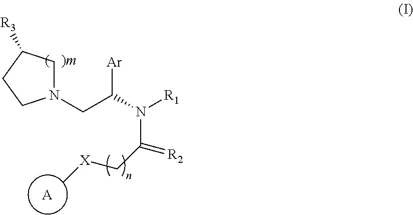

### EMBODIMENT(S) OF THE INVENTION

An embodiment of the present invention provides novel compounds of the general formula (I), their tautomeric forms, their enantiomers, their diastereoisomers, their stereoisomers, their pharmaceutically acceptable salts, and pharmaceutical compositions containing them or their suitable mixtures.

In a further embodiment of the present invention is provided pharmaceutical composition containing compounds of the general formula (I), their tautomeric forms, their enantiomers, their diastereoisomers, their stereoisomers, their pharmaceutically acceptable salts, or their mixtures in combination with suitable carriers, solvents, diluents and other media normally employed in preparing such compositions.

In a still further embodiment is provided the use of novel compounds of the present invention as KOR agonist, by administering a therapeutically effective and non-toxic amount of compounds of general formula (I) or their pharmaceutically acceptable compositions to the mammals.

In yet another embodiment are provided processes for the preparation of the compounds of formula (I) or their pharmaceutically acceptable salts, tautomers and enantiomeric forms.

List of abbreviations used in the description of the preparation of the compounds of the present invention:
AC: Adenylyl cyclase
ACN: Acetonitrile
BOP: Benzotriazole-1-yl-oxy-tris-(dimethylamino)-phoshphonium hexafluorophosphate
CNS: Central nervous system
DCC: N,N′-Dicyclohexyl carbodiimide
DMAP: Dimethyl amino pyridine
EDCI: (1-ethyl-3-(3-dimethylaminopropyl)carbodiimide hydrochloride
GPCRs: G protein-coupled receptors
δ: Delta
HOBt: 1-hydroxy benzotriazole
IBD: Inflammatory bowel disease
IBS: Irritable bowel syndrome
κ: Kappa
KOR: Kappa (κ) opioid receptors
LiAlH₄: Lithium aluminum hydride
μ: Mu
NaBH₄: Sodium borohydride
NMM: N-methyl morpholine
PNS: Peripheral nervous system

### DESCRIPTION OF THE INVENTION

Accordingly, the present invention relates to compounds of the general formula (I) represented below & includes their pharmaceutically acceptable salts

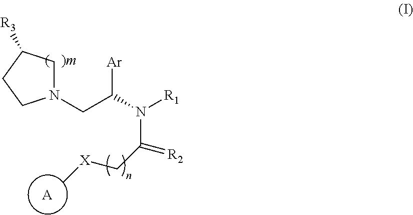

wherein:
R₁ represents hydrogen, optionally substituted groups selected from C₁₋₆ alkyl, aryl or arylalkyl;
   Wherein each of these groups, whenever applicable, is further substituted with hydroxy, halo, cyano, amino, (C₁₋₆)alkylamino, C(O)NH(C₁₋₆)alkyl groups;
R₂═O or NH;
R₃ is independently selected from hydroxyl, halogen, hydroxylalkyl, alkoxy, amino, C₁₋₄ alkyl, Aryl, heteroaryl, cyano;
m represents 0, 1 & 2; n represents 0, 1 & 2;
X═O or S;
‘Ar’ represents optionally substituted groups selected from aryl, heteroaryl, heterocyclyl, cycloalkylaryl, or cycloalkyl groups; wherein each of these groups, whenever applicable, is further substituted with hydroxy, (C₁₋₄)alkoxy, halo, cyano, amino, (C₁₋₆)alkylamino, nitro, COO(C₁₋₄)alkyl, S(O)n, S(O)nNH₂, S(O)nNH(C₁₋₆)alkyl, C(O); C(O)NH(C₁₋₆)alkyl, —O(CH₂)m-O—(CH₂)m-OH groups, wherein, n=1-2 and m=1-8;
‘A’ represents an optionally substituted rings selected form

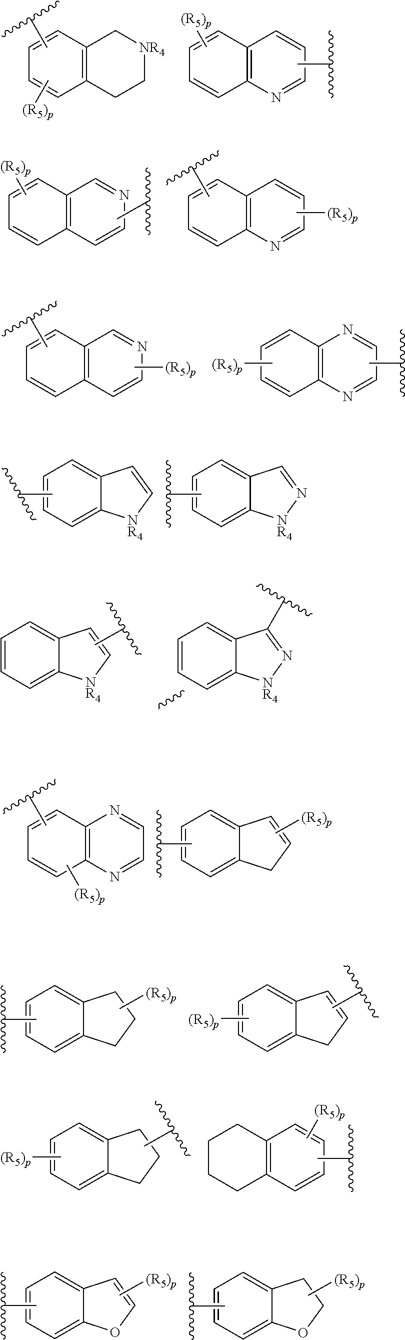

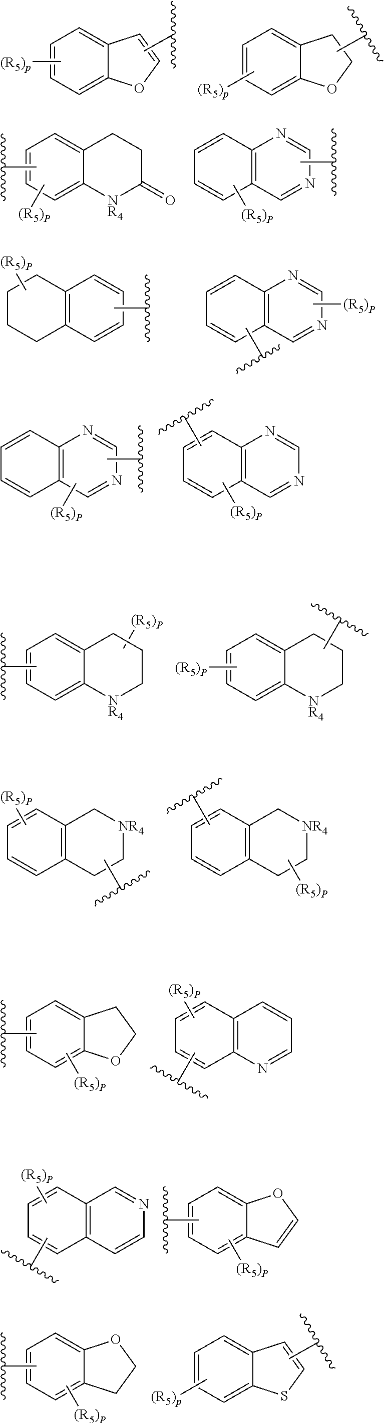

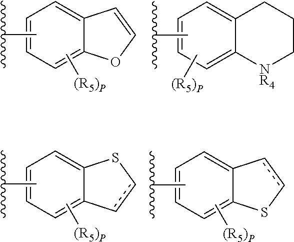

wherein R₄ at each occurrence is independently selected from guanidino, alkyl, haloalkyl, aryl, arylalkyl, heterocyclyl, heteroaryl, —SO₂Rₐ, —SO₂NHRₐ, —COR_{b}, —COOR_{b}, —NHCOOR_{b}. R₅ independently selected from cyano, hydroxyl, halogen, guanidino, alkyl, haloalkyl, aryl, arylalkyl, heterocyclyl, heteroaryl, —NHRₐ, —NHSO₂Rₐ, —SO₂Rₐ, —SO₂NHRₐ, —COR_{b}, —COOR_{b}, —NHCOOR_{b}, —O(CH₂)m-O—(CH₂)m-OH groups, wherein, m=1-8; ‘p’ represents integer from 0-4;
wherein, Rₐ & R_{b}, in each occurrence, is independently selected from hydrogen, alkyl or aryl;

In a preferred embodiment, the groups, radicals described above may be selected from:

“Alkyl”, as well as other groups having the prefix “alk”, such as alkoxy and alkanoyl, means carbon chain which may either be linear or branched, and combinations thereof, unless the carbon chain is defined otherwise. Examples of alkyl group include but not limited to methyl, ethyl, propyl, isopropyl, butyl, sec-butyl, tert.-butyl, pentyl, hexyl etc. Where the specified number of carbon atoms permits e.g. from C₃₋₁₀, the term alkyl also includes cycloalkyl groups, and combinations of linear or branched alkyl chains combined with cycloalkyl structures. When no number of carbon atoms is specified, C₁₋₆ is intended.

“Alkenyl” means carbon chains which contain at least one carbon-carbon double bond, and which may be linear or branched or combinations thereof, unless the carbon chain is defined otherwise. Examples of alkenyl include but not limited to vinyl, allyl, isopropenyl, hexenyl, pentenyl, heptenyl, 1-propenyl, 2-butenyl, 2-methyl-2-butenyl etc. Where the specified number of carbon atoms permits, e. g., from C₅₋₁₀, the term alkenyl also includes cycloalkenyl groups and combinations of linear, branched and cyclic structures. When no number of carbon atoms is specified, C(₂₋₆) is intended.

“Alkynyl” means carbon chains which contain at least one carbon-carbon triple bond, and which may be linear or branched or combinations thereof. Examples of alkynyl include ethynyl, propargyl, 3-methyl-1-pentynyl etc. When no number of carbon atoms is specified, C(₂₋₆) is intended.

“Cycloalkyl” is the subset of alkyl and means saturated carbocyclic ring having a specified number of carbon atoms, preferably 3-6 carbon atoms. Examples of cycloalkyl include cyclopropyl, cyclobutyl, cyclopentyl, cyclohexyl, cycloheptyl etc. A cycloalkyl group generally is monocyclic unless otherwise stated. Cycloalkyl groups are saturated unless and otherwise stated.

The “alkoxy” refers to the straight or branched chain alkoxides of the number of carbon atoms specified.

The term “alkylamino” refers to straight or branched alkylamines of the number of carbon atoms specified.

“Aryl” means a mono- or polycyclic aromatic ring system containing carbon ring atoms. The preferred aryls are monocyclic or bicyclic 6-10 membered aromatic ring systems. Phenyl and naphthyl are preferred aryls.

“Heterocycle” and “heterocyclyl” refer to saturated or unsaturated non-aromatic rings or ring systems containing at least one heteroatom selected from O, S, N further including the oxidized forms of sulfur, namely SO & SO₂. Examples of heterocycles include tetrahydrofuran (THF), dihydrofuran, 1,4-dioxane, morpholine, 1,4-dithiane, piperazine, piperidine, 1,3-dioxolane, imidazoline, imidazolidine, pyrrolidine, pyrroline, tetrahydropyran, dihydropyran, oxathiolane, dithiolane, 1,3-dioxane, 1,3-dithiane, oxathiane, thiomorpholine etc.

“Heteroaryl” means an aromatic or partially aromatic heterocycle that contains at least one ring heteroatom selected from O, S and N. Heteroaryls thus include heteroaryls fused to the other kinds of rings, such as aryls, cycloalkyls, and heterocycles that are not aromatic. Examples of heteroaryl groups include; pyrrolyl, isoxazolyl, isothiazolyl, pyrazolyl, pyridyl, oxazolyl, oxadiazolyl, thiadiazolyl, thiazolyl, imidazolyl, triazolyl, tetrazolyl, furyl, triazinyl, thienyl, pyrimidyl, benzisoxazolyl, benzoxazolyl, benzthiazolyl, benzothiadiazolyl, dihydrobenzofuranyl, indolinyl, pyridazinyl, indazolyl, isoindolyl, dihydrobenzothienyl, indolinyl, pyridazinyl, indazolyl, isoindolyl, dihydrobenzothienyl, indolizinyl, cinnolinyl, phthalazinyl, quinazolinyl, napthyridinyl, carbazolyl, benzodioxolyl, quinoxalinyl, purinyl, furazanyl, isobenzylfuranyl, benzimidazolyl, benzofuranyl, benzothienyl, quinolyl, indolyl, isoquinolyl, dibenzofuranyl etc. For heterocyclyl and heteroaryl groups, rings and ring systems containing from 3-15 carbon atoms are included, forming 1-3 rings.

“Halogen” refers to fluorine, chlorine, bromine, iodine. Chlorine and fluorine are generally preferred.

Suitable groups and substituents on the groups may be selected from those described anywhere in the specification.

The term “substituted,” as used herein, means that any one or more hydrogens on the designated atom is replaced with a selection from the indicated group, provided that the designated atom's normal valency is not exceeded, and that the substitution results in a stable compound. The term “substituted,” as used herein, means that any one or more hydrogens on the designated atom is replaced with a selection from the indicated group, provided that the designated atom's normal valency is not exceeded, and that the substitution results in a stable compound.

“Pharmaceutically acceptable salts” refer to derivatives of the disclosed compounds wherein the parent compound is modified by making acid or base salts thereof. Examples of pharmaceutically acceptable salts include, but are not limited to, mineral or organic acid salts of the basic residues. Such conventional non-toxic salts include, but are not limited to, those derived from inorganic and organic acids selected from 1, 2-ethanedisulfonic, 2-acetoxybenzoic, 2-hydroxyethanesulfonic, acetic, ascorbic, benzenesulfonic, benzoic, bicarbonic, carbonic, citric, edetic, ethane disulfonic, ethane sulfonic, fumaric, glucoheptonic, gluconic, glutamic, glycolic, glycollyarsanilic, hexylresorcinic, hydrabamic, hydrobromic, hydrochloric, hydroiodide, hydroxymaleic, hydroxynaphthoic, isethionic, lactic, lactobionic, lauryl sulfonic, maleic, malic, mandelic, methanesulfonic, napsylic, nitric, oxalic, pamoic, pantothenic, phenylacetic, phosphoric, polygalacturonic, propionic, salicyclic, stearic, subacetic, succinic, sulfamic, sulfanilic, sulfuric, tannic, tartaric, and toluenesulfonic.

The term ‘optional’ or ‘optionally’ means that the subsequent described event or circumstance may or may not occur, and the description includes instances where the event or circumstance occur and instances in which it does not. For example, ‘optionally substituted alkyl’ means either ‘alkyl’ or ‘substituted alkyl’. Further an optionally substituted group means unsubstituted.

Unless otherwise stated in the specification, structures depicted herein are also meant to include compounds which differ only in the presence of one or more isotopically enriched atoms.

Particularly useful compounds may be selected from but not limited to the following;

TABLE 1
List of compounds as KOR agonist

| S. No | Structures | IUPAC Name |
|---|---|---|
| 1 | 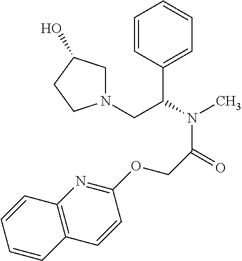 | N-((S)-2-((S)-3-hydroxypyrrolidin-1-yl)-1-phenylethyl)- N-methyl-2-(quinolin-2-yloxy)acetamide |
| 2 | 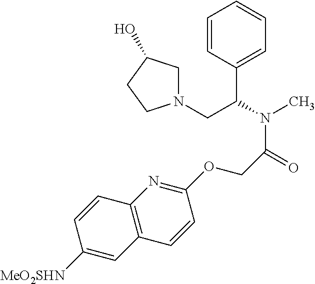 | N-((S)-2-((S)-3-hydroxypyrrolidin-1-yl)-1-phenylethyl)- N-methyl-2-((6-methylsulfonamido)quinolin-2- yloxy)acetamide |
| 3 | 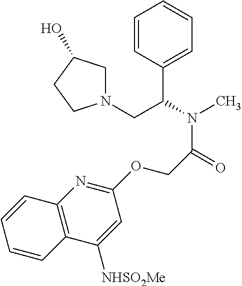 | N-((S)-2-((S)-3-hydroxypyrrolidin-1-yl)-1-phenylethyl)- N-methyl-2-((4-methylsulfonamido)quinolin-2- yloxy)acetamide |
| 4 | 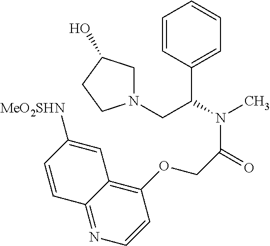 | N-((S)-2-((S)-3-hydroxypyrrolidin-1-yl)-1-phenylethyl)- N-methyl-2-((6-methylsulfonamido)quinolin-4- yloxy)acetamide |
| 5 | 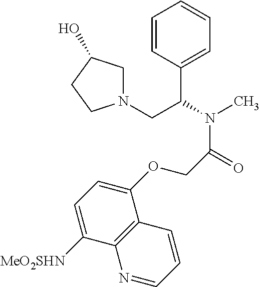 | N-((S)-2-((S)-3-hydroxypyrrolidin-1-yl)-1-phenylethyl)- N-methyl-2-((8-methylsulfonamido)quinolin-5- yloxy)acetamide |
| 6 | 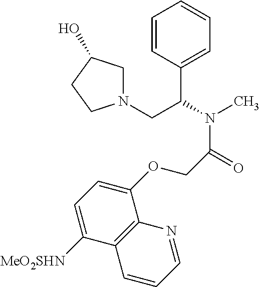 | N-((S)-2-((S)-3-hydroxypyrrolidin-1-yl)-1-phenylethyl)- N-methyl-2-((5-methylsulfonamido)quinolin-8- yloxy)acetamide |
| 7 | 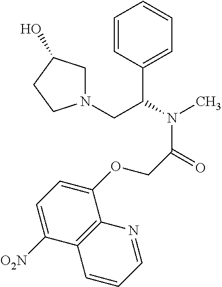 | N-((S)-2-((S)-3-hydroxypyrrolidin-1-yl)-1-phenylethyl)- N-methyl-2-((5-nitroquinolin-8-yl)oxy)acetamide |
| 8 | 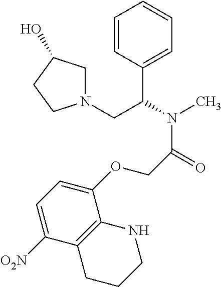 | N-((S)-2-((S)-3-hydroxypyrrolidin-1-yl)-1-phenylethyl)- N-methyl-2-((5-nitroquinolin-1,2,3,4-tetrahydroquinolin- 8-yl)oxy)acetamide |
| 9 | 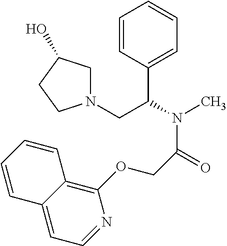 | N-((S)-2-((S)-3-hydroxypyrrolidin-1-yl)-1-phenylethyl)- 2-(isoquinolin-1-yloxy)-N-methylacetamide |
| 10 | 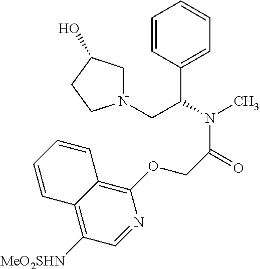 | N-((S)-2-((S)-3-hydroxypyrrolidin-1-yl)-1-phenylethyl)- N-methyl-2-((4-methylsulfonamido)isoquinolin-1- yloxy)acetamide |
| 11 | 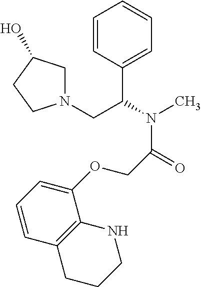 | N-((S)-2-((S)-3-hydroxypyrrolidin-1-yl)-1-phenylethyl)- N-methyl-2-(1,2,3,4-tetrahydroquinolin-8- yloxy)acetamide |
| 12 | 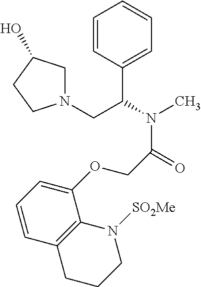 | N-((S)-2-((S)-3-hydroxypyrrolidin-1-yl)-1-phenylethyl)- N-methyl-2-((1-(methylsulfonyl)-1,2,3,4- tetrahydroquinolin-8-yl)oxy)acetamide |
| 13 | 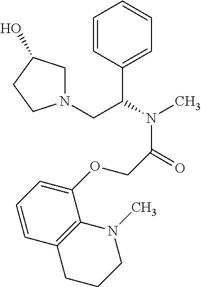 | N-((S)-2-((S)-3-hydroxypyrrolidin-1-yl)-1-phenylethyl)- N-methyl-2-((1-methyl-1,2,3,4-tetrahydroquinolin-8- yl)oxy)acetamide |
| 14 | 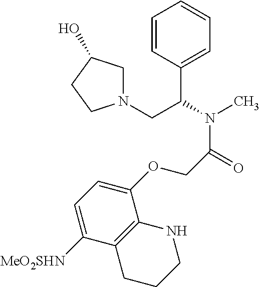 | N-((S)-2-((S)-3-hydroxypyrrolidin-1-yl)-1-phenylethyl)- N-methyl-2-((5-(methylsulfonamido)-1,2,3,4- tetrahydroquinolin-8-yl)oxy)acetamide |
| 15 | 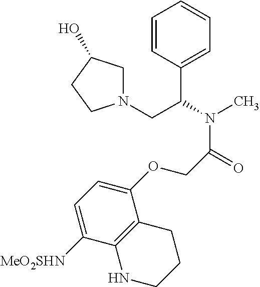 | N-((S)-2-((S)-3-hydroxypyrrolidin-1-yl)-1-phenylethyl)- N-methyl-2-((8-(methylsulfonamido)-1,2,3,4- tetrahydroquinolin-5-yl)oxy)acetamide |
| 16 | 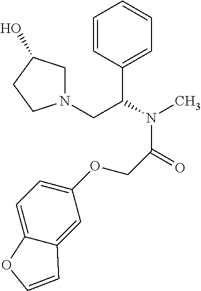 | 2-((benzofuran-5-yloxy)-N-((S)-2-((S)-3-hydroxy- pyrrolidin-1-yl)-1-phenylethyl)-N-methylacetamide |
| 17 | 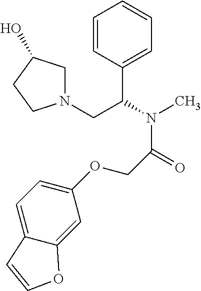 | 2-(benzofuran-6-yloxy)-N-((S)-2-((S)-3- hydroxypyrrolidin-1-yl)-1-phenylethyl)-N- methylacetamide |
| 18 | 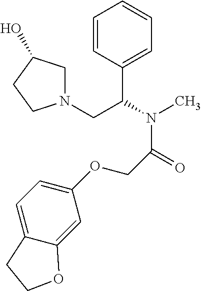 | 2-((2,3-dihydrobenzofuran-6-yl)oxy)-N-((S)-2-((S)-3- hydroxypyrrolidin-1-yl)-1-phenylethyl)-N- methylacetamide |
| 19 | 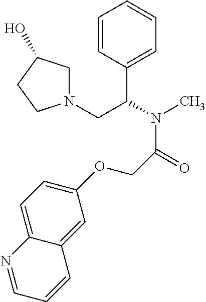 | N-((S)-2-((S)-3-hydroxypyrrolidin-1-yl)-1-phenylethyl)- N-methyl-2-(quinolin-6-yloxy)acetamide |
| 20 | 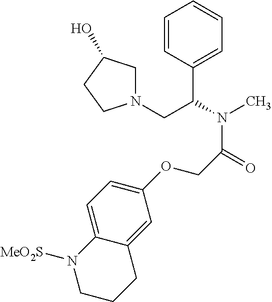 | N-((S)-2-((S)-3-hydroxypyrrolidin-1-yl)-1-phenylethyl)- N-methyl-2-((1-(methylsulfonyl)-1,2,3,4- tetrahydroquinolin-6-yl)oxy)acetamide |
| 21 | 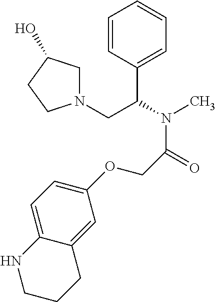 | N-((S)-2-((S)-3-hydroxypyrrolidin-1-yl)-1-phenylethyl)- N-methyl-2-((1,2,3,4-tetrahydroquinolin-6- yl)oxy)acetamide |
| 22 | 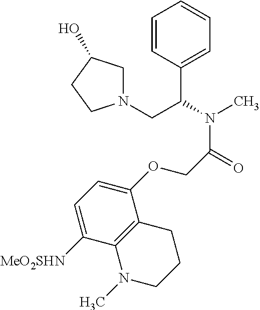 | N-((S)-2-((S)-3-hydroxypyrrolidin-1-yl)-1-phenylethyl)- N-methyl-2-((1-methyl-8-(methylsulfonamido)-1,2,3,4- tetrahydroquinolin-5-yl)oxy)acetamide |
| 23 | 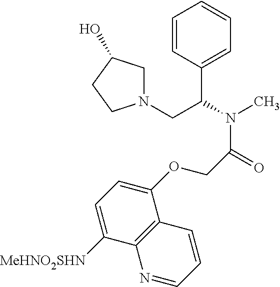 | N-((S)-2-((S)-3-hydroxypyrrolidin-1-yl)-1-phenylethyl)- N-methyl-2-((8-((N-methylsulfamoyl)amino)quinolin-5- yl)oxy)-acetamide |
| 24 | 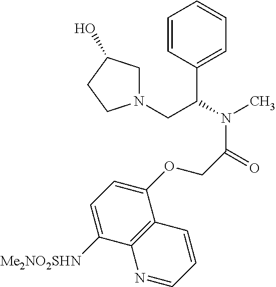 | 2-((8-((N,N-dimethylsulfamoyl)amino)quinolin-5- yl)oxy)-N-((S)-2-((S)-3-hydroxypyrrolidin-1-yl)-1- phenylethyl)-N-methyl-acetamide |
| 25 | 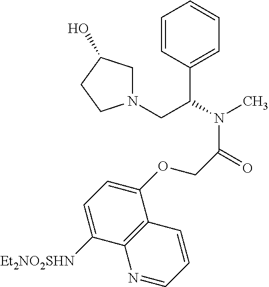 | 2-((8-((N,N-diethylsulfamoyl)amino)quinolin-5-yl)oxy)- N-((S)-2-((S)-3-hydroxypyrrolidin-1-yl)-1-phenylethyl)- N-methyl-acetamide |
| 26 | 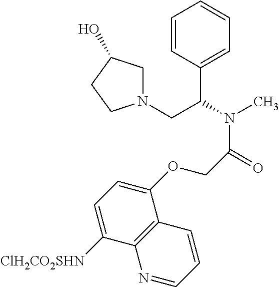 | 2-((8-((chloromethylsulfonamido)quinolin-5-yl)oxy)-N- ((S)-2-((S)-3-hydroxypyrrolidin-1-yl)-1-phenylethyl)-N- methyl-acetamide |
| 27 | 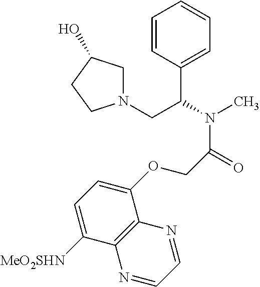 | N-((S)-2-((S)-3-hydroxypyrrolidin-1-yl-)-1-phenylethyl)- N-methyl-2-((8-(methylsulfonamido)quinoxain-5- yl)oxy)acetamide |
| 28 | 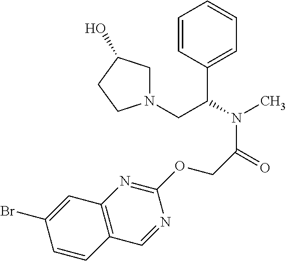 | 2-((7-bromoquinazolin-2-yl)oxy)-N-((S)-2-((S)-3- hydroxypyrrolidin-1-yl)-1-phenylethyl)-N- methylacetamide |
| 29 | 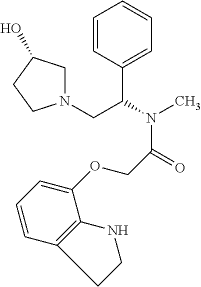 | N-((S)-2-((S)-3-hydroxypyrrolidin-1-yl-)-1-phenylethyl)- 2-(indolin-7-yloxy)-N-methylacetamide |
| 30 | 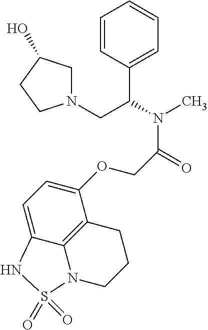 | 2-((2,2-dioxido-1,4,5,6-tetrahydro- [1,2,5]thiadiazolo[4,3,2-ij]quinolin-7-yl)oxy-N-((S)-2- ((S)-3-hydroxypyrrolidin-1-yl)-1-phenylethyl)-N- methylacetamide |
| 31 | 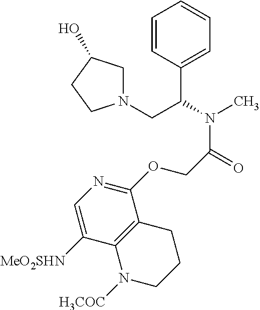 | 2-((l-acetyl-8-(methylsulfonamido)-1,2,3,4- tetrahydroquinolin-5-yl)oxy)-N-((S)-2-((S)-3- hydroxypyrrolidin-1-yl)-1-phenylethyl)-N- methylacetamide |
| 32 | 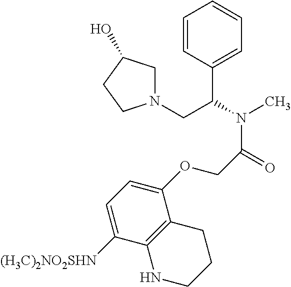 | 2-((8-((N,N-dimethylsulfamoyl)amino)-1,2,3,4- tetrahydroquinolin-5-yl)oxy)-N-((S)-2-((S)-3- hydroxypyrrolidin-1-yl)-1-phenylethyl)-N- methylacetamide |
| 33 | 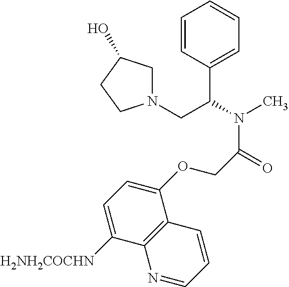 | 2-((8-(2-aminoacetamido)quinolin-5-yl)oxy)-N-((S)-2- ((S)-3-hydroxypyrrolidin-1-yl)-1-phenylethyl)-N- methylacetamide |
| 34 | 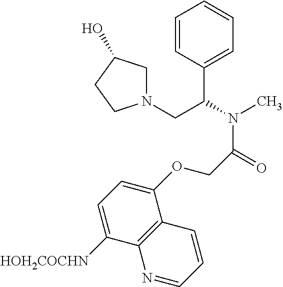 | 2-((8-(2-hydroxyacetamido)quinolin-5-yl)oxy)-N-((S)-2- ((S)-3-hydroxypyrrolidin-1-yl)-1-phenylethyl)-N- methylacetamide |
| 35 | 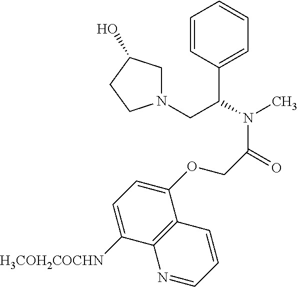 | N-((S)-2-((S)-3-hydroxypyrrolidin-1-yl)-1-phenylethyl)- 2-((8-(2-methoxyacetamido)quinolin-5-yl)oxy)-N- methylacetamide |
| 36 | 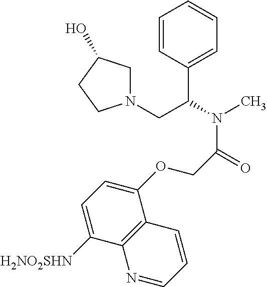 | N-((S)-2-((S)-3-hydroxypyrrolidin-1-yl)-1-phenylethyl)- N-methyl-2-((8-sulfonamido)quinolin-5-yloxy)acetamide |
| 37 | 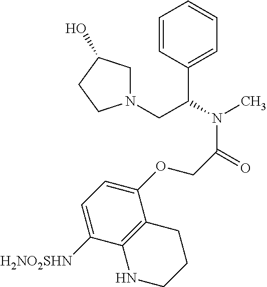 | N-((S)-2-((S)-3-hydroxypyrrolidin-1-yl)-1-phenylethyl)- N-methyl-2-((8-sulfonamido)-1,2,3,4-tetrahydroquinolin- 5-yloxy)acetamide |
| 38 | 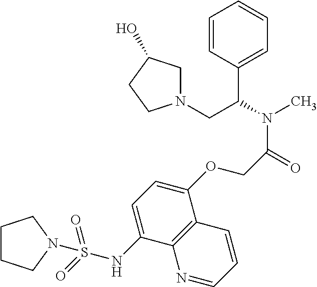 | N-((S)-2-((S)-3-hydroxypyrrolidin-1-yl)-1-phenylethyl)- N-methyl-2-((8-(pyrrolidine-1-sulfonamido)quinolin-5- yloxy)acetamide |
| 39 | 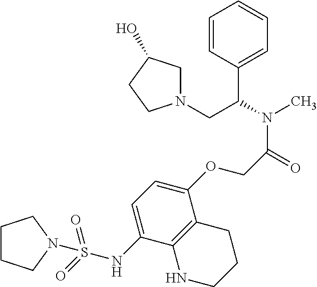 | N-((S)-2-((S)-3-hydroxypyrrolidin-1-yl)-1-phenylethyl)- N-methyl-2-((8-(pyrrolidine-1-sulfonamido)-1,2,3,4- tetrahydroquinolin-5-yloxy)acetamide |
| 40 | 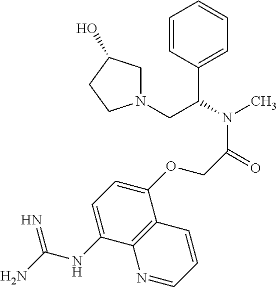 | 2-((8-guanidinoquinolin-5-yl)oxy)-N-((S)-2-((S)-3- hydroxypyrrolidin-1-yl)-1-phenylethyl)-N- methylacetamide |
| 41 | 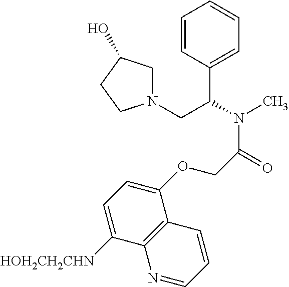 | 2-((8-(2-hydroxyethyl)amino)quinolin-5-yl)oxy)-N-((S)- 2-((S)-3-hydroxypyrrolidin-1-yl)-1-phenylethyl)-N- methylacetamide |
| 42 | 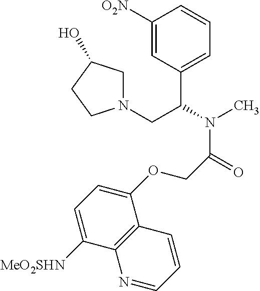 | N-((S)-2-((S)-3-hydroxypyrrolidin-1-yl)-1-(3- nitrophenyl)ethyl)-N-methyl-2-((8- (methylsulfonamido)quinolin-5-yl)oxy) acetamide |
| 43 | 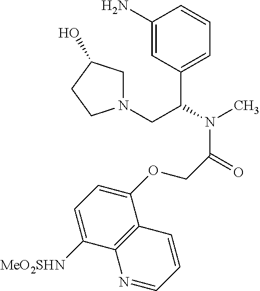 | N-((S)-1-(3-aminophenyl)-2-((S)-3-hydroxypyrrolidin-1- yl)ethyl)-N-methyl-2-((8-(methylsulfonamido)quinolin- 5-yl)oxy) acetamide |
| 44 | 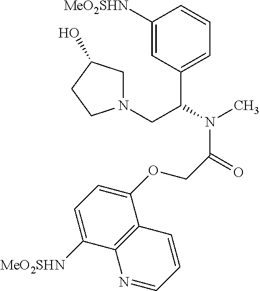 | N-((S)-2-((S)-3-hydroxypyrrolidin-1-yl)-1- (methylsulfonamido)phenyl)ethyl)-N-methyl-2-((8- (methylsulfonamido)quinolin-5-yl)oxy) acetamide |
| 45 | 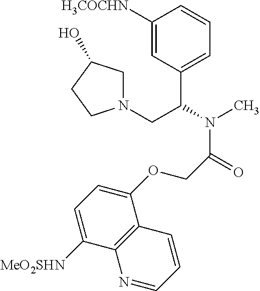 | N-((S)-1-(3-acetamidophenyl)-2-((S)-3- hydroxypyrrolidin-1-yl)ethyl)-N-methyl-2-((8- (methylsulfonamido)quinolin-5-yl)oxy) acetamide |
| 46 | 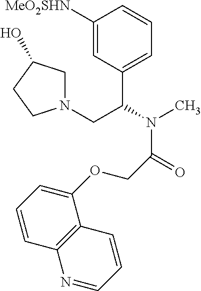 | N-((S)-2-((S)-3-hydroxypyrrolidin-1-yl)-1- (methylsulfonamido)phenyl)ethyl)-N-methyl-2- (quinolin-5-yl)oxy) acetamide |
| 47 | 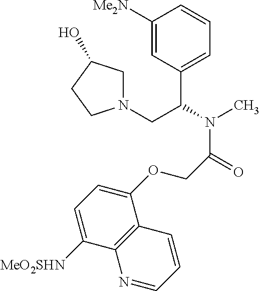 | N-((S)-1-(3-dimethylaminophenyl)-2-((S)-3- hydroxypyrrolidin-1-yl)ethyl)-N-methyl-2-((8- (methylsulfonamido)quinolin-5-yl)oxy) acetamide |
| 48 | 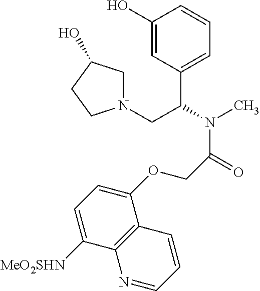 | N-((S)-1-(3-hydroxyphenyl)-2-((S)-3-hydroxypyrrolidin- 1-yl)ethyl)-N-methyl-2-((8- (methylsulfonamido)quinolin-5-yl)oxy) acetamide |
| 49 | 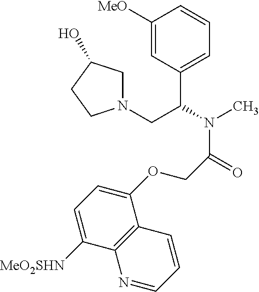 | N-((S)-2-((S)-3-hydroxypyrrolidin-1-yl)-1-(3- methoxyphenyl)ethyl)-N-methyl-2-((8- (methylsulfonamido)quinolin-5-yl)oxy) acetamide |
| 50 | 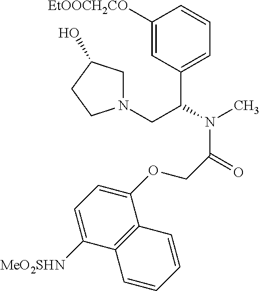 | Ethyl-2-(3((S)-2-((S)-3-hydroxypyrrolidin-1-yl)-1-N- methyl-2-((8-(methylsulfonamido)quinolin-5- yl)oxy)acetamido)ethyl)phenoxy)acetate |
| 51 | 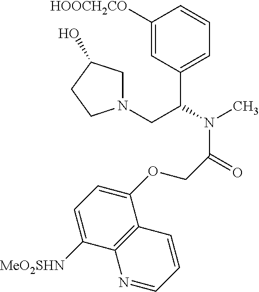 | 2-(3((S)-2-((S)-3-hydroxypyrrolidin-1-yl)-1-N-methyl-2- ((8-(methylsulfonamido)quinolin-5- yl)oxy)acetamido)ethyl)phenoxy)acetic acid |
| 52 | 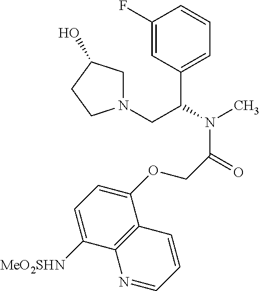 | N-((S)-1-(3-fluorophenyl)-2-((S)-3-hydroxypyrrolidin-1- yl)ethyl)-N-methyl-2-((8-(methylsulfonamido)quinolin- 5-yl)oxy) acetamide |
| 53 | 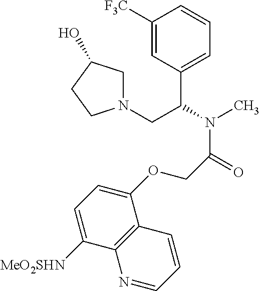 | N-((S)-2-((S)-3-hydroxypyrrolidin-1-yl)-1-(3- (trifluoromethyl)phenyl)ethyl)-N-methyl-2-((8- (methylsulfonamido)quinolin-5-yl)oxy) acetamide |
| 54 | 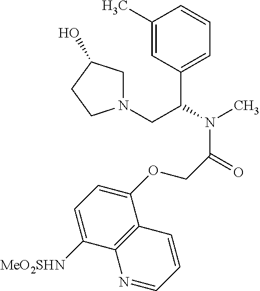 | N-((S)-2-((S)-3-hydroxypyrrolidin-1-yl)-1-(3- methyl)ethyl)-N-methyl-2-((8- (methylsulfonamido)quinolin-5-yl)oxy)acetamide |
| 55 | 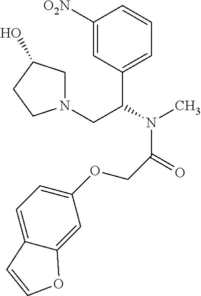 | 2-(benzofuran-6-yloxy)-N-((S)-2-((S)-3- hydroxypyrrolidin-1-yl)-1-(3-nitrophenyl)ethyl)-N- methylacetamide |
| 56 | 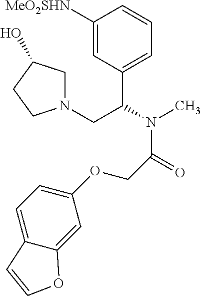 | 2-(benzofuran-6-yloxy)-N-((S)-2-((S)-3- hydroxypyrrolidin-1-yl)-1-(3- (methylsulfonamido)phenyl)ethyl)-N-methylacetamide |
| 57 | 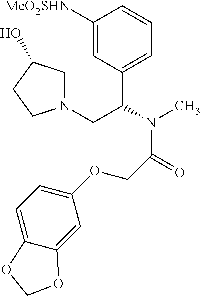 | 2-(benzo[d][1,3]dioxol-5-yloxy)-N-((S)-2-((S)-3- hydroxypyrrolidin-1-yl)-1-(3- (methylsulfonamido)phenyl)ethyl)-N-methylacetamide |
| 58 | 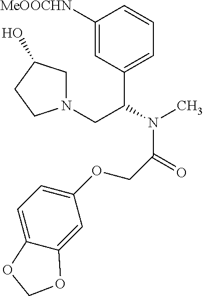 | Methyl-(3-((S)-1-(2-(benzo[d][1,3]dioxol-5-yloxy)-N- methylacetamido)-2-((S)-3-hydroxypyrrolidin-1-yl)- ethyl)phenyl)carbamate |

or a pharmaceutically acceptable salts of any of the compounds above.

The novel compounds of the present invention may be prepared using the reactions and techniques described below together with conventional techniques known to those skilled in the art of organic synthesis or variations thereof as appreciated by those skilled in the art.

The reactions are performed in solvents appropriate to the reagents and materials employed and are suitable for the transformations being effected. Preferred methods include, but not limited to those described below, where all symbols are as defined earlier unless and otherwise defined below.

The compounds of the formula (I) can be prepared as described in Scheme-1, along with suitable modifications/variations, which are well within the scope of a person skilled in the art.
Step i: Substituted L-Phenylgylcine (1) can be reacting with protecting agent such as ethylchloroformate in presence of a mild base such as sodium bicarbonate, under suitable conditions of solvent and temperature, to yield a compound (2).
Step ii: Condensation of compound (2) with Compound (3) using suitable coupling agents such as EDCI/HOBt, HATU, BOP, PyBOP, DCC/HOBt, and the like, in a suitable solvent such as DCM, DMF and the like, in the presence or absence of base like DMAP, DIPEA can yield a compound (4).
Step iii: Compound (5) can be obtained by reduction of the compound (4) using suitable reducing agents such as LiAlH₄, NaBH₄ and the like, under suitable conditions of solvent and temperature.
Step iv: Condensation of compound (5) with compound (6) using suitable coupling agents such as EDCI/HOBt, HATU, BOP, PyBOP, DCC/HOBt, and the like, in a suitable solvent such as DCM, DMF and the like, in the presence or absence of base like DMAP, DIPEA can yield a compound of formula-I.

The examples and preparations provided below further illustrate and exemplify the compounds of the present invention and methods of preparing such compounds. It is to be understood that the scope of the present invention is not limited in any way by the scope of the following examples and preparations. In the following examples molecules with a single chiral center, unless otherwise noted, exist as a racemic mixture. Those molecules with two or more chiral centers, unless otherwise noted, exist as a racemic mixture of diastereomers. Single enantiomers/diastereomers may be obtained by methods known to those skilled in the art.

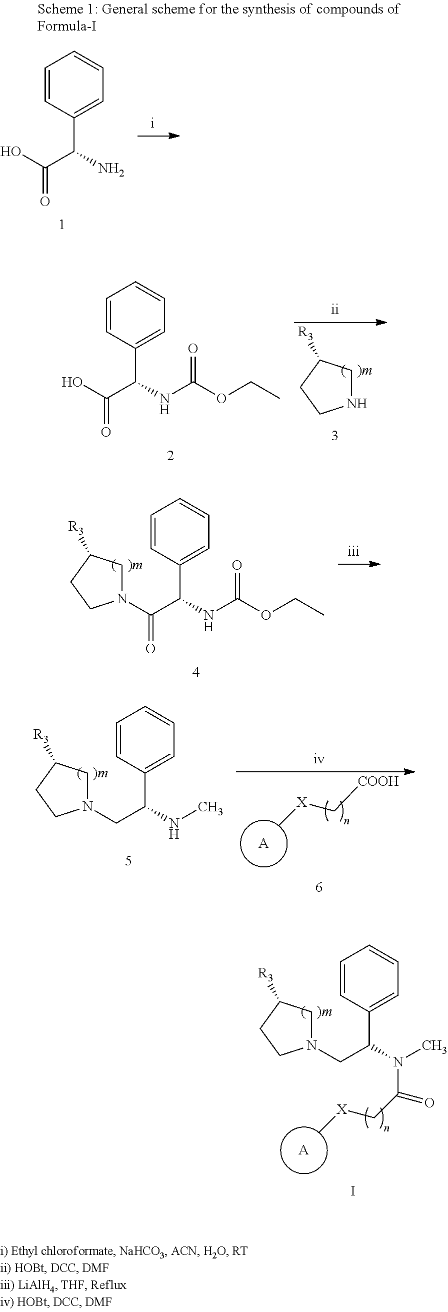

### Synthesis of Compound 9: N—((S)-2-((S)-3-hydroxypyrrolidin-1-yl)-1-phenylethyl)-2-(iso-quinolin-1-yloxy)-N-methylacetamide

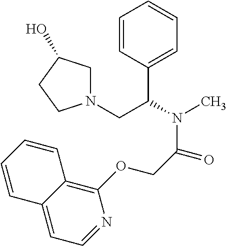

### Step-i: Synthesis of (S)-2-((ethoxycarbonyl)amino)-2-phenylacetic acid

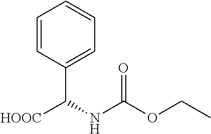

To a solution of L-Phenyl glycine (2.5 g, 16.7 mmol), in aqueous NaOH (3N; 10 ml), ethyl chloroformate (1.2 ml, 10.5 mmol) was added and the reaction mixture was stirred for 20 min. at 0-5° C. The second portion of aqueous NaOH (3N; 7 ml), ethyl chloroformate (1.2 ml, 10.5 mmol) was added and the reaction mixture was stirred for 2 h at 0-5° C. The mixture was filtered and washed with diethyl ether. The aqueous layer was acidified with 6N HCl (pH-4) to get the solid (S)-2-((ethoxycarbonyl)amino)-2-phenylacetic acid (3.4 g, 92% yield).

¹H NMR: (DMSO-d6, 400 MHz): 12.80 (brs, 1H), 7.87 (d, 1H, J=8.4 Hz), 7.41-7.28 (m, 5H), 5.12 (d, 1H, J=8.4 Hz), 4.04-3.98 (m, 2H), 1.20 (t, 1H, J=14.4 Hz); ESI-MS: (+ve mode) 224.0 (M+H)⁺ (100%); HPLC: 99.6%.

### Step-ii: Synthesis of ethyl((S)-2-((S)-3-hydroxypyrrolidin-1-yl)-2-oxo-1-phenylethyl)carbamate

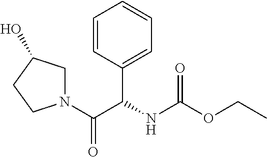

To a solution of (S)-2-((ethoxycarbonyl)amino)-2-phenylacetic acid (2.0 g, 8.96 mmol) in THF (20 ml), NMM (1.0 ml, 8.96 mmol), and ethyl chloroformate (1.1 ml, 8.96 mmol) was added at 0-5° C. The reaction mixture was stirred for 20 min., at 0-5° C. To it, S-pyrrolidinol (0.78 g, 8.96 mmol) was added and the mixture was stirred for 24 h at 25-30° C. The reaction mixture was diluted with DCM and washed with water. Organic layer was dried over Na₂SO₄ and evaporated to get ethyl((S)-2-((S)-3-hydroxypyrrolidin-1-yl)-2-oxo-1-phenylethyl) as a pale yellow oil (2.2 g, 85% yield).

¹H NMR: (DMSO-d6, 400 MHz): 7.53-7.26 (m, 5H), 5.02 (m, 1H), 4.27-4.16 (m, 2H), 3.69-3.62 (m, 1H), 3.38-3.32 (m, 2H), 3.25-3.16 (m, 2H), 1.77-1.64 (m, 2H), 1.10 (t, 1H, J=14.0 Hz); ESI-MS: (+ve mode) 293.05 (M+H)⁺ (100%).

### Step-iii: Synthesis of (S)-1-((S)-2-(methylamino)-2-phenylethyl)pyrrolidin-3-01

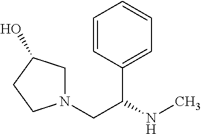

LAH (0.78 g, 20.5 mmol) was dissolved in dry THF (10 ml) at 0-5° C., followed by addition of ((S)-2-((S)-3-hydroxypyrrolidin-1-yl)-2-oxo-1-phenylethyl) (1.5 g, 5.13 mmol) in THF (10 ml). The reaction mixture was refluxed for 3 h, quenched with saturated Na₂CO₃ solution and triturated with ethyl acetate. The reaction mixture was filtered through celite and organic layer was concentrated under reduce pressure to get the (S)-1-((S)-2-(methylamino)-2-phenylethyl)pyrrolidin-3-ol, as a pale yellow oil. (1.02, 90% yield).

¹H NMR: (DMSO-d6, 400 MHz): 7.37-7.27 (m, 4H), 7.26-7.23 (m, 1H), 4.80-4.68 (m, 1H), 4.27-4.16 (m, 1H), 3.69-3.62 (m, 2H), 2.78-2.62 (m, 2H), 2.52 (s, 3H), 2.25-2.12 (m, 2H), 1.77-1.64 (m, 2H); ESI-MS: (+ve mode) 221.05 (M+H)⁺ (100%).

### Step-iv: N—((S)-2-((S)-3-hydroxypyrrolidin-1-yl)-1-phenylethyl)-2-(iso-quinolin-1-yloxy)-N-methylacetamide

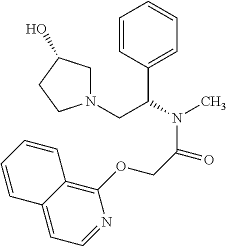

To a solution of 2-(isoquinolin-1-yloxy)aceticacid (0.46 g, 2.27 mmol), dissolved in DCM (5 ml), HOBt (0.3 g, 2.27 mmol) and DCC (0.47 g, 2.27 mmol) was added at 25-30° C. The mixture was stirred for 10 min., and to it (S)-1-((S)-2-(methylamino)-2-phenylethyl)pyrrolidin-3-ol (0.5 g, 2.27 mmol) was added. The reaction mixture was stirred for 24 h at 25-30° C., filtered and the filtrate was diluted with DCM. Organic layer was washed with saturated NaHCO₃ solution and brine, dried over Na₂SO₄ and evaporated to get the crude product. Crude product was purified by column chromatography using 0 to 2% MeOH in DCM as an eluent system, to get the title compound as a white solid (0.72 g, 78% yield).

¹H NMR: (DMSO-d6, 400 MHz): 8.23-8.21 (m, 1H), 7.75-7.74 (m, 1H), 7.73-7.71 (m, 1H), 7.68-7.66 (m, 1H), 7.63-7.61 (m, 2H), 7.54-7.52 (m, 2H), 7.44-7.42 (m, 1H), 7.38-7.35 (m, 2H), 6.13-6.09 (m, 1H), 5.23-5.21 (m, 1H), 4.45-4.41 (m, 1H), 4.23-4.19 (m, 2H), 4.20-4.12 (m, 3H), 3.73-3.69 (m, 2H), 2.85 (d, 3H), 2.33-2.28 (m, 2H); ESI-MS: (+ve mode) 406.05 (M+H)⁺ (100%); HPLC: 98.36%.

Compounds of the present invention can be isolated either as free amine form or as a salt corresponding to the acid used such as trifluoroacetic acid, hydrochloric acid, hydrobromic acid, oxalic acid, maleic acid, fumeric acid, succinic acid, p-toluene sulfonic acid or benzene sulfonic acid. The compounds can be purified where ever required, by recrystallization, trituration, precipitation, preparative thin layer chromatography, flash chromatography or by preparative HPLC method.

The compounds of the present invention can be used either alone or in combination with one or more therapeutic agents or pharmaceutically acceptable salts thereof. Such use will depend on the condition of the patient being treated and is well within the scope of a skilled practitioner.

The invention is further illustrated by the following non-limiting examples which describe the preferred way of carrying out the present invention. These are provided without limiting the scope of the present invention in any way.

¹H NMR spectral data given in the examples (vide infra) are recorded using a 400 MHz spectrometer (Bruker AVANCE-400) and reported in δ scale. Until and otherwise mentioned the solvent used for NMR is CDCl₃ using TMS as the internal standard.

### Compound 1: N—((S)-2-((S)-3-hydroxypyrrolidin-1-yl)-1-phenylethyl)-N-methyl-2-(quinolin-2-yloxy)acetamide

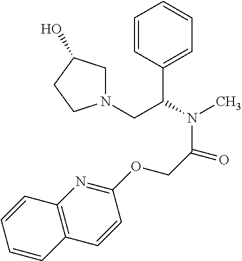

¹H NMR: (DMSO-d6, 400 MHz): 7.97 (d, 1H, J=8.8 Hz), 7.75-7.723 (m, 3H), 7.48-7.44 (m, 1H), 7.38-7.28 (m, 3H), 7.28-7.23 (m, 3H), 6.68-6.62 (m, 1H), 6.12-6.08 (m, 1H), 5.58 (s, 1H), 5.40 (dd, 1H), 5.32 (dd, 1H), 4.41 (dd, 1H), 3.72-3.66 (m, 2H), 3.56-3.52 (m, 2H), 3.37 (m, 1H), 2.95 (s, 3H), 2.39-2.29 (m, 1H), 1.90-1.84 (m, 1H); ESI-MS: (+ve mode) 406.05 (M+H)⁺ (100%); HPLC: 97.13%.

### Compound 2: N—((S)-2-((S)-3-hydroxypyrrolidin-1-yl)-1-phenylethyl)-N-methyl-2-((6-methyl-sulfonamido)quinolin-2-yloxy)acetamide

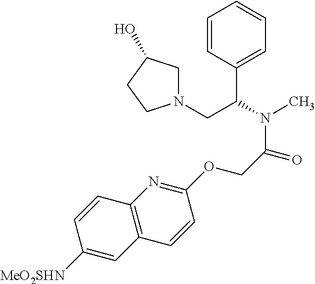

¹H NMR: (DMSO-d6, 400 MHz): 9.77 (s, 1H), 7.97 (d, 1H, J=9.6 Hz), 7.58 (d, 1H, J=8.6 Hz), 7.47-7.41 (m, 2H), 7.39-7.29 (m, 3H), 7.27-7.24 (m, 2H), 6.68 (d, 1H, J=12.0 Hz), 6.22-6.12 (m, 1H), 5.47 (dd, 1H), 5.25-5.12 (m, 2H), 4.51-4.38 (m, 1H), 4.20-4.14 (m, 1H), 3.75-3.64 (m, 2H), 3.32-3.18 (m, 1H), 2.96 (s, 3H), 2.80 (s, 3H), 2.33-2.28 (m, 1H), 2.26-1.95 (m, 1H), 1.91-1.80 (m, 1H); ESI-MS: (+ve mode) 499.30 (M+H)⁺ (100%); HPLC: 96.98%.

### Compound 3: N—((S)-2-((S)-3-hydroxypyrrolidin-1-yl)-1-phenylethyl)-N-methyl-2-((4-methyl-sulfonamido)quinolin-2-yloxy)acetamide

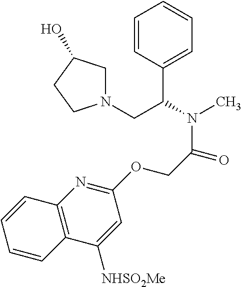

¹H NMR: (DMSO-d6, 400 MHz): 9.79 (s, 1H), 7.97 (d, 1H, J=9.6 Hz), 7.58 (d, 1H, J=8.6 Hz), 7.47-7.41 (m, 2H), 7.38-7.29 (m, 3H), 7.27-7.24 (m, 2H), 6.70 (d, 1H, J=12.0 Hz), 6.22-6.12 (m, 1H), 5.47 (dd, 1H), 5.25-5.12 (m, 2H), 4.51-4.38 (m, 1H), 4.20-4.14 (m, 1H), 3.75-3.64 (m, 2H), 3.32-3.18 (m, 1H), 2.96 (s, 3H), 2.80 (s, 3H), 2.33-2.28 (m, 1H), 2.26-1.95 (m, 1H), 1.91-1.80 (m, 1H); ESI-MS: (+ve mode) 499.40 (M+H)⁺ (100%); HPLC: 98.25%.

### Compound 4: N—((S)-2-((S)-3-hydroxypyrrolidin-1-yl)-1-phenylethyl)-N-methyl-2-((6-methyl-sulfonamido)quinolin-4-yloxy)acetamide

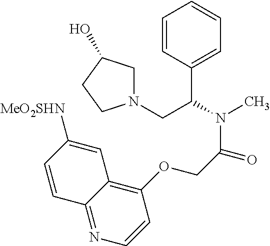

¹H NMR: (DMSO-d6, 400 MHz): 9.76 (s, 1H), 7.94 (d, 1H, J=9.7 Hz), 7.63 (d, 1H, J=8.8 Hz), 7.47-7.41 (m, 2H), 7.38-7.29 (m, 3H), 7.27-7.24 (m, 2H), 6.70 (d, 1H, J=12.2 Hz), 6.22-6.12 (m, 1H), 5.47 (dd, 1H), 5.28-5.14 (m, 2H), 4.51-4.38 (m, 1H), 4.20-4.14 (m, 1H), 3.75-3.64 (m, 2H), 3.32-3.18 (m, 1H), 2.96 (s, 3H), 2.80 (s, 3H), 2.33-2.28 (m, 1H), 2.26-1.95 (m, 1H), 1.91-1.80 (m, 1H); ESI-MS: (+ve mode) 499.43 (M+H)⁺ (100%); HPLC: 98.88%.

### Compound 5: N—((S)-2-((S)-3-hydroxypyrrolidin-1-yl)-1-phenylethyl)-N-methyl-2-((8-methyl-sulfonamido)quinolin-5-yloxy)acetamide

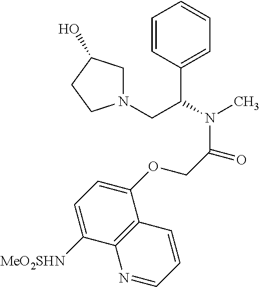

¹H NMR: (DMSO-d6, 400 MHz): 9.22 (d, 1H, J=7.2 Hz), 8.99-8.98 (m, 1H), 8.66-8.64 (m, 1H), 7.67-7.65 (m, 1H), 7.64-7.63 (m, 1H), 7.57-7.41 (m, 3H), 7.40-7.32 (m, 2H), 6.14-6.11 (m, 1H), 5.29-5.21 (m, 2H), 4.43-4.36 (m, 2H), 4.20-4.12 (m, 3H), 3.90-3.86 (m, 2H), 3.73-3.69 (m, 2H), 2.99 (s, 3H), 2.83 (d, 3H), 2.33-2.28 (m, 1H); ESI-MS: (+ve mode) 499.25 (M+H)⁺ (100%); HPLC: 98.14%.

### Compound 6: N—((S)-2-((S)-3-hydroxypyrrolidin-1-yl)-1-phenylethyl)-N-methyl-2-((5-methyl-sulfonamido)quinolin-8-yloxy)acetamide

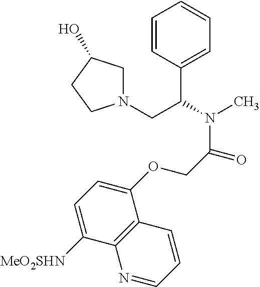

¹H NMR: (DMSO-d6, 400 MHz): 9.26 (d, 1H, J=7.4 Hz), 8.98-8.96 (m, 1H), 8.66-8.64 (m, 1H), 7.67-7.65 (m, 1H), 7.64-7.63 (m, 1H), 7.57-7.41 (m, 3H), 7.42-7.34 (m, 2H), 6.14-6.11 (m, 1H), 5.29-5.21 (m, 2H), 4.43-4.36 (m, 2H), 4.20-4.12 (m, 3H), 3.90-3.86 (m, 2H), 3.73-3.69 (m, 2H), 2.98 (s, 3H), 2.85 (d, 3H), 2.33-2.28 (m, 1H); ESI-MS: (+ve mode) 499.34 (M+H)⁺ (100%); HPLC: 97.15%.

### Compound 7: N—((S)-2-((S)-3-hydroxypyrrolidin-1-yl)-1-phenylethyl)-N-methyl-2-((5-nitro-quinolin-8-yl)oxy)acetamide

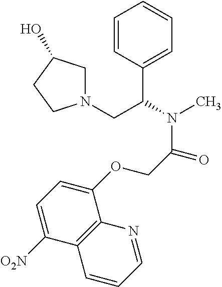

¹H NMR: (DMSO-d6, 400 MHz): 9.32 (d, 1H, J=7.8 Hz), 8.88-8.84 (m, 1H), 8.66-8.64 (m, 1H), 7.67-7.65 (m, 1H), 7.64-7.63 (m, 1H), 7.57-7.41 (m, 3H), 7.42-7.34 (m, 2H), 6.14-6.11 (m, 1H), 5.29-5.21 (m, 2H), 4.43-4.36 (m, 2H), 4.20-4.12 (m, 3H), 3.90-3.86 (m, 2H), 3.73-3.69 (m, 2H), 2.85 (d, 3H), 2.33-2.28 (m, 1H); ESI-MS: (+ve mode) 451.92 (M+H)⁺ (100%); HPLC: 98.23%.

### Compound 8: N—((S)-2-((S)-3-hydroxypyrrolidin-1-yl)-1-phenylethyl)-N-methyl-2-((5-nitro-quinolin-1,2,3,4-tetrahydroquinolin-8-yl)oxy)acetamide

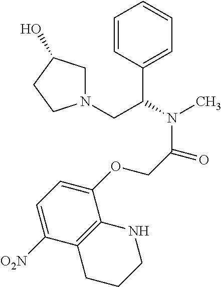

¹H NMR: (DMSO-d6, 400 MHz): 7.40-7.38 (m, 2H), 7.37-7.35 (m, 1H), 7.26-7.25 (m, 2H), 7.14-7.12 (m, 2H), 6.11-6.09 (m, 1H), 5.45-5.42 (m, 2H), 5.11-5.09 (m, 2H), 4.50-4.42 (m, 2H), 4.18-4.15 (m, 3H), 3.81-3.78 (m, 3H), 3.58-3.56 (m, 2H), 2.89-2.87 (m, 3H), 2.80 (s, 3H); ESI-MS: (+ve mode) 454.95 (M+H)⁺ (100%); HPLC: 96.83%.

### Compound 9: N—((S)-2-((S)-3-hydroxypyrrolidin-1-yl)-1-phenylethyl)-2-(iso-quinolin-1-yloxy)-N-methylacetamide

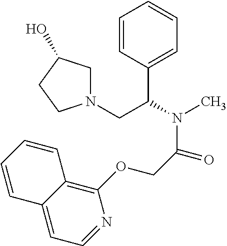

¹H NMR: (DMSO-d6, 400 MHz): 8.23-8.21 (m, 1H), 7.75-7.74 (m, 1H), 7.73-7.71 (m, 1H), 7.68-7.66 (m, 1H), 7.63-7.61 (m, 2H), 7.54-7.52 (m, 2H), 7.44-7.42 (m, 1H), 7.38-7.35 (m, 2H), 6.13-6.09 (m, 1H), 5.23-5.21 (m, 1H), 4.45-4.41 (m, 1H), 4.23-4.19 (m, 2H), 4.20-4.12 (m, 3H), 3.73-3.69 (m, 2H), 2.85 (d, 3H), 2.33-2.28 (m, 2H); ESI-MS: (+ve mode) 406.05 (M+H)⁺ (100%); HPLC: 98.36%.

### Compound 10: N—((S)-2-((S)-3-hydroxypyrrolidin-1-yl)-1-phenylethyl)-N-methyl-2-((4-methyl-sulfonamido)isoquinolin-1-yloxy)acetamide

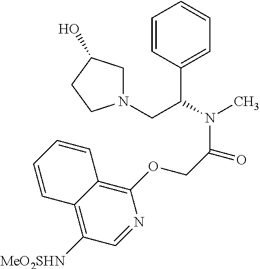

¹H NMR: (DMSO-d6, 400 MHz): 9.26 (d, 1H, J=7.4 Hz), 8.99-8.98 (m, 1H), 8.66-8.64 (m, 1H), 7.67-7.65 (m, 1H), 7.64-7.63 (m, 1H), 7.57-7.41 (m, 3H), 7.40-7.32 (m, 2H), 6.14-6.11 (m, 1H), 5.29-5.21 (m, 2H), 4.43-4.36 (m, 2H), 4.20-4.12 (m, 3H), 3.90-3.86 (m, 2H), 3.73-3.69 (m, 2H), 2.96 (s, 3H), 2.83 (d, 3H), 2.33-2.28 (m, 1H); ESI-MS: (+ve mode) 499.34 (M+H)⁺ (100%); HPLC: 97.45%.

### Compound 11: N—((S)-2-((S)-3-hydroxypyrrolidin-1-yl)-1-phenylethyl)-N-methyl-2-(1,2,3,4-tetra hydroquinolin-8-yloxy)acetamide

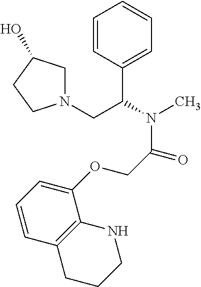

¹H NMR: (DMSO-d6, 400 MHz): 7.44-7.42 (m, 2H), 7.39-7.37 (m, 2H), 7.28-7.26 (m, 2H), 7.14-7.12 (m, 2H), 6.11-6.09 (m, 1H), 5.45-5.42 (m, 2H), 5.11-5.09 (m, 2H), 4.50-4.42 (m, 2H), 4.18-4.15 (m, 3H), 3.81-3.78 (m, 3H), 3.58-3.56 (m, 2H), 2.89-2.87 (m, 3H), 2.84 (s, 3H); ESI-MS: (+ve mode) 410.45 (M+H)⁺ (100%); HPLC: 97.55%.

### Compound 12: N—((S)-2-((S)-3-hydroxypyrrolidin-1-yl)-1-phenylethyl)-N-methyl-2-((1-(methyl-sulfonyl)-1,2,3,4-tetrahydroquinolin-8-yl)oxy)acetamide

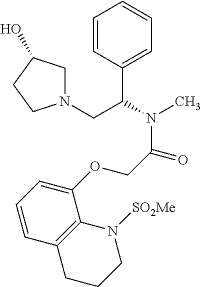

¹H NMR: (DMSO-d6, 400 MHz): 7.42-7.40 (m, 2H), 7.36-7.35 (m, 1H), 7.28-7.26 (m, 2H), 7.14-7.12 (m, 2H), 6.11-6.09 (m, 1H), 5.45-5.42 (m, 2H), 5.11-5.09 (m, 2H), 4.50-4.42 (m, 2H), 4.18-4.15 (m, 3H), 3.81-3.78 (m, 3H), 3.58-3.56 (m, 2H), 2.89-2.87 (m, 3H), 3.12 (s, 3H), 2.79 (s, 3H); ESI-MS: (+ve mode) 488.05 (M+H)⁺ (100%); HPLC: 99.15%.

### Compound 13: N—((S)-2-((S)-3-hydroxypyrrolidin-1-yl)-1-phenylethyl)-N-methyl-2-((1-methyl-1,2,3,4-tetrahydroquinolin-8-yl)oxy)acetamide

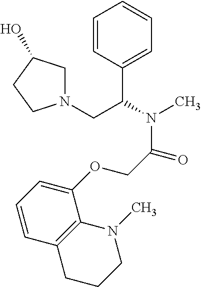

¹H NMR: (DMSO-d6, 400 MHz): 7.42-7.40 (m, 2H), 7.36-7.35 (m, 1H), 7.28-7.26 (m, 2H), 7.14-7.12 (m, 2H), 6.11-6.09 (m, 1H), 5.45-5.42 (m, 2H), 5.11-5.09 (m, 2H), 4.50-4.42 (m, 2H), 4.18-4.15 (m, 3H), 3.81-3.78 (m, 3H), 3.58-3.56 (m, 2H), 2.89-2.87 (m, 3H), 2.84 (s, 3H), 2.79 (s, 3H); ESI-MS: (+ve mode) 424.05 (M+H)⁺ (100%); HPLC: 95.99%.

### Compound 14: N—((S)-2-((S)-3-hydroxypyrrolidin-1-yl)-1-phenylethyl)-N-methyl-2-((5-(methyl-sulfonamido)-1,2,3,4-tetrahydroquinolin-8-yl)oxy)acetamide

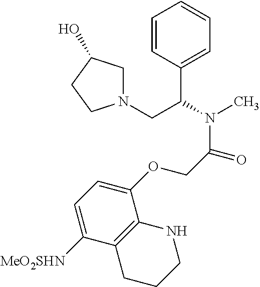

¹H NMR: (DMSO-d6, 400 MHz): 8.73 (s, 1H), 7.41-7.39 (m, 3H), 7.36-7.35 (m, 2H), 6.86-6.80 (m, 2H), 6.11-6.09 (m, 1H), 5.45-5.42 (m, 2H), 5.11-5.09 (m, 2H), 4.50-4.42 (m, 2H), 4.18-4.15 (m, 3H), 3.81-3.78 (m, 3H), 3.58-3.56 (m, 2H), 2.89-2.87 (m, 3H), 3.12 (s, 3H), 2.79 (s, 3H); ESI-MS: (+ve mode) 503.25 (M+H)⁺ (100%); HPLC: 96.23%.

### Compound 15: N—((S)-2-((S)-3-hydroxypyrrolidin-1-yl)-1-phenylethyl)-N-methyl-2-((8-(methyl-sulfonamido)-1,2,3,4-tetrahydroquinolin-5-yl)oxy)acetamide

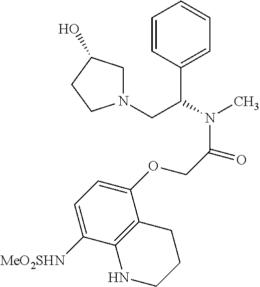

¹H NMR: (DMSO-d6, 400 MHz): 8.44 (s, 1H), 7.42-7.34 (m, 3H), 7.23-7.21 (m, 2H), 6.83-6.81 (m, 1H), 6.15-6.12 (m, 2H), 5.45-5.42 (m, 2H), 5.11-5.09 (m, 2H), 4.50-4.42 (m, 2H), 4.18-4.15 (m, 3H), 3.81-3.78 (m, 3H), 3.58-3.56 (m, 2H), 2.89-2.87 (m, 3H), 3.12 (s, 3H), 2.79 (s, 3H); ESI-MS: (+ve mode) 503.15 (M+H)⁺ (100%); HPLC: 99.23%.

### Compound 16: 2-((benzofuran-5-yloxy)-N—((S)-2-((S)-3-hydroxy-pyrrolidin-1-yl)-1-phenyl-ethyl)-N-methylacetamide

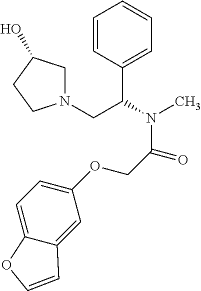

¹H NMR: (DMSO-d6, 400 MHz): 7.94 (d, 1H, J=6.8 Hz), 7.48-7.47 (m, 1H), 7.44-7.39 (m, 3H), 7.38-7.36 (m, 3H), 6.96-6.94 (m, 1H), 6.85-6.83 (m, 1H), 6.11-6.07 (m, 1H), 5.29-5.21 (m, 1H), 4.98-4.90 (m, 2H), 4.20-4.12 (m, 2H), 3.90-3.86 (m, 2H), 3.73-3.69 (m, 2H), 2.75 (d, 3H), 2.33-2.28 (m, 2H); ESI-MS: (+ve mode) 395.05 (M+H)⁺ (100%); HPLC: 98.73%.

### Compound 17: 2-(benzofuran-6-yloxy)-N—((S)-2-((S)-3-hydroxypyrrolidin-1-yl)-1-phenylethyl)-N-methylacetamide

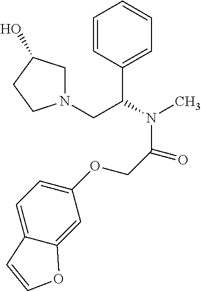

¹H NMR: (DMSO-d6, 400 MHz): 7.85 (d, 1H, J=6.9 Hz), 7.48-7.47 (m, 1H), 7.44-7.39 (m, 3H), 7.38-7.36 (m, 3H), 6.96-6.94 (m, 1H), 6.85-6.83 (m, 1H), 5.75-5.72 (m, 1H), 5.29-5.21 (m, 1H), 4.95-4.89 (m, 2H), 4.20-4.12 (m, 2H), 3.90-3.86 (m, 2H), 3.73-3.69 (m, 2H), 2.77 (d, 3H), 2.33-2.28 (m, 2H); ESI-MS: (+ve mode) 395.30 (M+H)⁺ (100%); HPLC: 96.64%.

### Compound 18: 2-((2,3-dihydrobenzofuran-6-yl)oxy)-N—((S)-2-((S)-3-hydroxypyrrolidin-1-yl)-1-phenylethyl)-N-methylacetamide

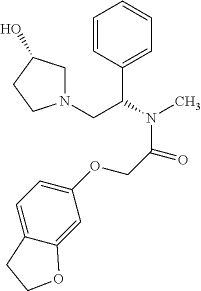

¹H NMR: (DMSO-d6, 400 MHz): 7.42-7.33 (m, 3H), 7.24-7.11 (m, 2H), 7.08 (d, 3H, J=8.0 Hz), 6.43-6.40 (m, 2H), 6.10-6.06 (m, 1H), 5.75-5.72 (m, 1H), 4.91-4.89 (m, 1H), 4.86-4.84 (m, 1H), 4.49-4.46 (m, 3H), 4.09-4.03 (m, 1H), 3.90-3.86 (m, 2H), 3.73-3.69 (m, 2H), 2.69 (d, 3H), 2.33-2.28 (m, 2H); ESI-MS: (+ve mode) 397.00 (M+H)⁺ (100%); HPLC: 99.02%.

### Compound 19: N—((S)-2-((S)-3-hydroxypyrrolidin-1-yl)-1-phenylethyl)-N-methyl-2-(quinolin-6-yloxy)acetamide

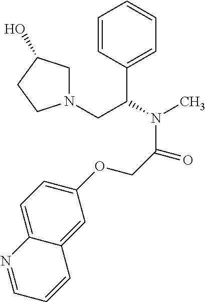

¹H NMR: (DMSO-d6, 400 MHz): 8.86-8.84 (m, 1H), 8.47-8.45 (m, 1H), 8.03-8.00 (m, 1H), 7.67-7.61 (m, 3H), 7.39-7.33 (m, 3H), 7.29-7.27 (m, 2H), 6.14-6.10 (m, 1H), 5.21-5.17 (m, 2H), 4.45-4.41 (m, 1H), 4.23-4.19 (m, 2H), 3.72-3.66 (m, 1H), 3.63-3.59 (m, 3H), 2.85 (d, 3H), 2.33-2.28 (m, 2H); ESI-MS: (+ve mode) 406.05 (M+H)⁺ (100%); HPLC: 97.27%.

### Compound 20: N—((S)-2-((S)-3-hydroxypyrrolidin-1-yl)-1-phenylethyl)-N-methyl-2-((1-(methyl-sulfonyl)-1, 2, 3, 4-tetrahydroquinolin-6-yl)oxy)acetamide

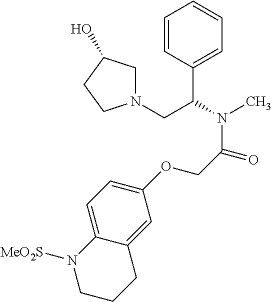

¹H NMR: (DMSO-d6, 400 MHz): 7.43-7.38 (m, 4H), 7.27-7.24 (m, 2H), 6.90-6.83 (m, 2H), 6.13-6.10 (m, 1H), 4.95-4.89 (m, 2H), 4.45-4.43 (m, 1H), 4.16-4.12 (m, 2H), 3.81-3.78 (m, 3H), 3.58-3.56 (m, 2H), 2.94 (s, 3H), 2.79 (s, 3H), 1.94-1.88 (m, 3H); ESI-MS: (+ve mode) 488.45 (M+H)⁺ (100%); HPLC: 97.38%.

### Compound 21: N—((S)-2-((S)-3-hydroxypyrrolidin-1-yl)-1-phenylethyl)-N-methyl-2-((1,2,3,4-tetrahydroquinolin-6-yl)oxy)acetamide

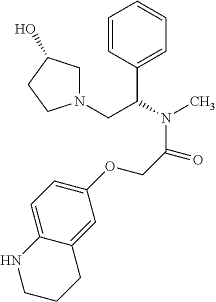

¹H NMR: (DMSO-d6, 400 MHz): 7.42-7.40 (m, 2H), 7.38-7.36 (m, 2H), 7.28-7.26 (m, 2H), 7.14-7.12 (m, 2H), 6.11-6.09 (m, 1H), 5.45-5.42 (m, 2H), 5.11-5.09 (m, 2H), 4.50-4.42 (m, 2H), 4.18-4.15 (m, 3H), 3.81-3.78 (m, 3H), 3.58-3.56 (m, 2H), 2.89-2.87 (m, 3H), 2.76 (s, 3H); ESI-MS: (+ve mode) 410.15 (M+H)⁺ (100%); HPLC: 94.81%.

### Compound 22: N—((S)-2-((S)-3-hydroxypyrrolidin-1-yl)-1-phenylethyl)-N-methyl-2-((1-methyl-8-(methylsulfonamido)-1, 2, 3, 4-tetrahydroquinolin-5-yl)oxy)acetamide

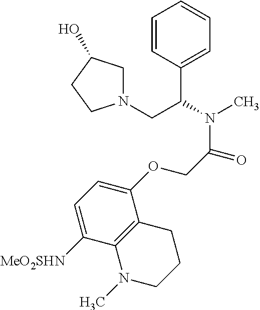

¹H NMR: (DMSO-d6, 400 MHz): 8.43 (d, 1H, J=8.6 Hz), 7.43-7.35 (m, 3H), 7.24-7.22 (m, 2H), 6.98-6.96 (m, 1H), 6.45 (m, 1H), 6.09-6.03 (m, 1H), 4.93-4.91 (m, 1H), 4.84-4.81 (m, 1H), 4.09-4.03 (m, 2H), 3.69-3.63 (m, 3H), 3.46-3.43 (m, 2H), 3.28-3.24 (m, 4H), 2.84 (s, 3H), 2.75 (s, 6H), 2.47-2.43 (m, 1H), 2.31-2.29 (m, 2H); ESI-MS: (+ve mode) 517.25 (M+H)⁺ (100%); HPLC: 97.34%.

### Compound 23: N—((S)-2-((S)-3-hydroxypyrrolidin-1-yl)-1-phenylethyl)-N-methyl-2-((8-((N-methylsulfamoyl)amino)quinolin-5-yl)oxy)-acetamide

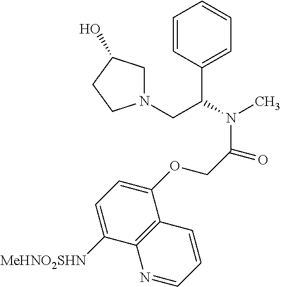

¹H NMR: (DMSO-d6, 400 MHz): 9.22 (d, 1H, J=7.2 Hz), 8.99 (s, 2H), 8.66-8.64 (m, 1H), 7.67-7.65 (m, 1H), 7.64-7.63 (m, 1H), 7.57-7.41 (m, 3H), 7.40-7.32 (m, 2H), 6.14-6.11 (m, 1H), 5.29-5.21 (m, 2H), 4.43-4.36 (m, 2H), 4.20-4.12 (m, 3H), 3.90-3.86 (m, 2H), 3.73-3.69 (m, 2H), 2.99 (s, 3H), 2.83 (d, 3H), 2.33-2.28 (m, 1H); ESI-MS: (+ve mode) 514.08 (M+H)⁺ (100%); HPLC: 99.05%.

### Compound 24: 2-((8-((N,N-dimethylsulfamoyl)amino)quinolin-5-yl)oxy)-N—((S)-2-((S)-3-hydroxypyrrolidin-1-yl)-1-phenylethyl)-N-methyl-acetamide

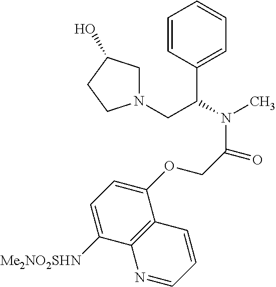

¹H NMR: (DMSO-d6, 400 MHz): 9.14 (d, 1H, J=8.8 Hz), 9.01 (d, 1H, J=8.6 Hz), 8.99 (m, 1H), 7.67-7.61 (m, 2H), 7.43-7.37 (m, 3H), 7.27-7.25 (m, 2H), 7.10-7.07 (m, 1H), 6.14-6.09 (m, 1H), 5.29-5.21 (m, 2H), 4.43-4.36 (m, 2H), 4.20-4.12 (m, 3H), 3.90-3.86 (m, 2H), 3.73-3.69 (m, 2H), 2.75 (s, 3H), 2.50 (d, 6H), 2.33-2.28 (m, 1H); ESI-MS: (+ve mode) 528.25 (M+H)⁺ (100%); HPLC: 96.87%.

### Compound 25: 2-((8-((N,N-diethylsulfamoyl)amino)quinolin-5-yl)oxy)-N—((S)-2-((S)-3-hydroxy pyrrolidin-1-yl)-1-phenylethyl)-N-methyl-acetamide

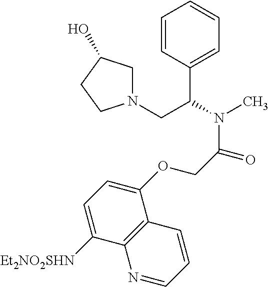

¹H NMR: (DMSO-d6, 400 MHz): 9.14 (d, 1H, J=8.8 Hz), 9.01 (d, 1H, J=8.6 Hz), 8.99 (m, 1H), 7.67-7.61 (m, 2H), 7.43-7.37 (m, 3H), 7.27-7.25 (m, 2H), 7.10-7.07 (m, 1H), 6.14-6.09 (m, 1H), 5.29-5.21 (m, 2H), 4.43-4.36 (m, 2H), 4.20-4.12 (m, 3H), 3.90-3.86 (m, 2H), 3.73-3.69 (m, 2H), 2.84-3.78 (m, 4H), 2.75 (s, 3H), 2.50 (d, 6H), 2.33-2.28 (m, 1H); ESI-MS: (+ve mode) 556.32 (M+H)⁺ (100%); HPLC: 97.05%.

### Compound 26: 2-((8-((chloromethylsulfonamido)quinolin-5-yl)oxy)-N—((S)-2-((S)-3-hydroxy pyrrolidin-1-yl)-1-phenylethyl)-N-methyl-acetamide

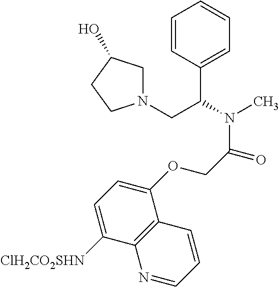

¹H NMR: (DMSO-d6, 400 MHz): 9.22 (d, 1H, J=7.8 Hz), 8.99-8.98 (m, 1H), 8.66-8.64 (m, 1H), 7.67-7.65 (m, 1H), 7.64-7.63 (m, 1H), 7.57-7.41 (m, 3H), 7.40-7.32 (m, 2H), 6.14-6.11 (m, 1H), 5.29-5.21 (m, 2H), 4.43-4.36 (m, 2H), 4.20-4.12 (m, 3H), 3.90-3.86 (m, 2H), 3.73-3.69 (m, 2H), 2.88 (s, 3H), 2.33-2.28 (m, 3H); ESI-MS: (+ve mode) 533.67 (M+H)⁺ (100%); HPLC: 97.01%.

### Compound 27: N—((S)-2-((S)-3-hydroxypyrrolidin-1-yl)-1-phenylethyl)-N-methyl-2-((8-(methyl-sulfonamido)quinoxain-5-yl)oxy)acetamide

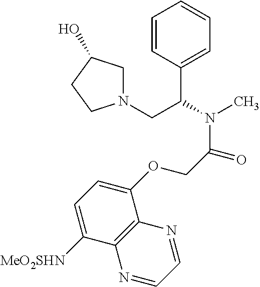

¹H NMR: (DMSO-d6, 400 MHz): 9.46 (d, 1H, J=7.6 Hz), 8.97 (d, 2H), 7.68-7.66 (m, 1H), 7.64-7.63 (m, 1H), 7.57-7.41 (m, 3H), 7.40-7.32 (m, 2H), 6.14-6.11 (m, 1H), 5.29-5.21 (m, 2H), 4.43-4.36 (m, 2H), 4.20-4.12 (m, 3H), 3.90-3.86 (m, 2H), 3.73-3.69 (m, 2H), 3.14 (s, 3H), 2.84 (d, 3H), 2.33-2.28 (m, 1H); ESI-MS: (+ve mode) 500.15 (M+H)⁺ (100%); HPLC: 97.56%.

### Compound 28: 2-((7-bromoquinazolin-2-yl)oxy)-N—((S)-2-((S)-3-hydroxypyrrolidin-1-yl)-1-phenylethyl)-N-methylacetamide

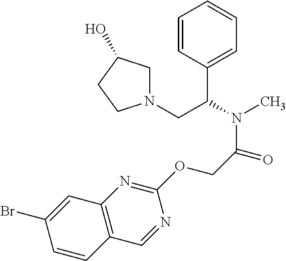

¹H NMR: (DMSO-d6, 400 MHz): 8.88 (d, 2H), 7.69-7.67 (m, 1H), 7.64-7.63 (m, 1H), 7.57-7.41 (m, 3H), 7.40-7.32 (m, 2H), 6.12-6.09 (m, 1H), 5.29-5.21 (m, 2H), 4.43-4.36 (m, 2H), 4.20-4.12 (m, 3H), 3.90-3.86 (m, 2H), 3.73-3.69 (m, 2H), 2.33-2.28 (m, 1H); ESI-MS: (+ve mode) 485.34 (M+H)⁺ (100%); HPLC: 98.34%.

### Compound 29: N—((S)-2-((S)-3-hydroxypyrrolidin-1-yl)-1-phenylethyl)-2-(indolin-7-yloxy)-N-methylacetamide

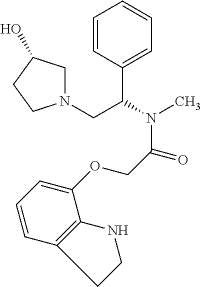

¹H NMR: (DMSO-d6, 400 MHz): 7.48-7.39 (m, 3H), 7.26-7.14 (m, 2H), 7.18 (d, 3H, J=8.0 Hz), 6.44-6.39 (m, 2H), 6.10-6.06 (m, 1H), 5.75-5.72 (m, 1H), 4.91-4.89 (m, 1H), 4.86-4.84 (m, 1H), 4.49-4.46 (m, 3H), 4.09-4.03 (m, 1H), 3.90-3.86 (m, 2H), 3.73-3.69 (m, 2H), 2.69 (d, 3H), 2.33-2.28 (m, 2H); ESI-MS: (+ve mode) 396.12 (M+H)⁺ (100%); HPLC: 97.77%.

### Compound 30: 2-((2, 2-dioxido-1,4,5,6-tetrahydro-[1,2,5]thiadiazolo[4,3,2-ij]quinolin-7-yl)oxy-N—((S)-2-((S)-3-hydroxypyrrolidin-1-yl)-1-phenylethyl)-N-methylacetamide

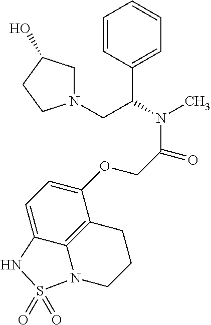

¹H NMR: (DMSO-d6, 400 MHz): 8.48 (s, 1H), 7.44-7.36 (m, 3H), 7.23-7.21 (m, 2H), 6.83-6.81 (m, 1H), 6.15-6.12 (m, 2H), 5.48-5.44 (m, 2H), 5.11-5.09 (m, 2H), 4.50-4.42 (m, 2H), 4.18-4.15 (m, 3H), 3.81-3.78 (m, 3H), 3.58-3.56 (m, 2H), 2.89-2.87 (m, 3H), 2.79 (s, 3H); ESI-MS: (+ve mode) 487.23 (M+H)⁺ (100%); HPLC: 98.53%.

### Compound 31: 2-((1-acetyl-8-(methylsulfonamido)-1,2,3,4-tetrahydroquinolin-5-yl)oxy)-N—((S)-2-((S)-3-hydroxypyrrolidin-1-yl)-1-phenylethyl)-N-methylacetamide

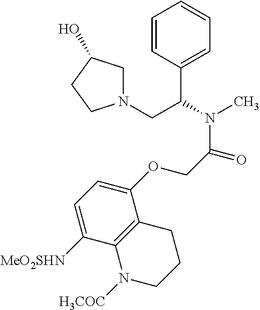

¹H NMR: (DMSO-d6, 400 MHz): 8.44 (s, 1H), 7.42-7.34 (m, 3H), 7.23-7.21 (m, 2H), 6.83-6.81 (m, 2H), 6.15-6.12 (m, 2H), 5.45-5.42 (m, 2H), 5.11-5.09 (m, 2H), 4.50-4.42 (m, 2H), 4.18-4.15 (m, 3H), 3.81-3.78 (m, 3H), 3.58-3.56 (m, 2H), 2.89-2.87 (m, 3H), 3.12 (s, 3H), 2.98 (s, 3H); ESI-MS: (+ve mode) 545.40 (M+H)⁺ (100%); HPLC: 98.76%.

### Compound 32: 2-((8-((N,N-dimethylsulfamoyl)amino)-1,2,3,4-tetrahydroquinolin-5-yl)oxy)-N—((S)-2-((S)-3-hydroxypyrrolidin-1-yl)-1-phenylethyl)-N-methylacetamide

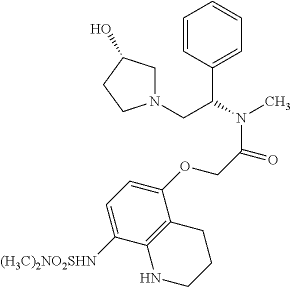

¹H NMR: (DMSO-d6, 400 MHz): 8.48 (s, 1H), 7.44-7.36 (m, 3H), 7.23-7.21 (m, 2H), 6.84-6.81 (m, 1H), 6.16-6.13 (m, 2H), 5.45-5.42 (m, 2H), 5.11-5.09 (m, 2H), 4.50-4.42 (m, 2H), 4.18-4.15 (m, 3H), 3.81-3.78 (m, 3H), 3.58-3.56 (m, 2H), 2.89-2.87 (m, 3H), 3.14 (s, 3H), 2.81 (s, 6H); ESI-MS: (+ve mode) 532.25 (M+H)⁺ (100%); HPLC: 97.22%.

### Compound 33: 2-((8-(2-aminoacetamido)quinolin-5-yl)oxy)-N—((S)-2-((S)-3-hydroxypyrrolidin-1-yl)-1-phenylethyl)-N-methylacetamide

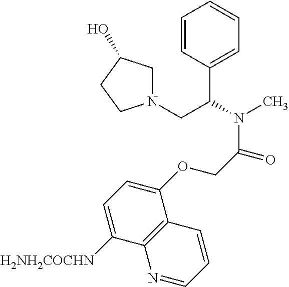

¹H NMR: (DMSO-d6, 400 MHz): 8.46 (s, 1H), 7.86-7.82 (m, 2H), 7.40-7.33 (m, 3H), 7.24-7.20 (m, 2H), 6.83-6.81 (m, 2H), 6.15-6.12 (m, 2H), 5.45-5.42 (m, 2H), 5.11-5.09 (m, 2H), 4.50-4.42 (m, 2H), 4.19-4.16 (m, 3H), 3.79-3.74 (m, 3H), 3.57-3.54 (m, 2H), 2.89-2.87 (m, 3H), 2.84-2.81 (m, 2H), 2.79 (s, 3H); ESI-MS: (+ve mode) 478.15 (M+H)⁺ (100%); HPLC: 98.44%.

### Compound 34: 2-((8-(2-hydroxyacetamido)quinolin-5-yl)oxy)-N—((S)-2-((S)-3-hydroxy-pyrrolidin-1-yl)-1-phenylethyl)-N-methylacetamide

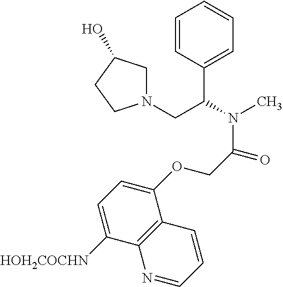

¹H NMR: (DMSO-d6, 400 MHz): 8.96 (s, 1H), 8.66 (s, 1H), 7.88-7.83 (m, 2H), 7.40-7.33 (m, 3H), 7.24-7.20 (m, 2H), 6.83-6.81 (m, 2H), 6.15-6.12 (m, 2H), 5.45-5.42 (m, 2H), 5.11-5.09 (m, 2H), 4.52-4.44 (m, 2H), 4.19-4.16 (m, 3H), 3.79-3.74 (m, 3H), 3.57-3.54 (m, 2H), 2.89-2.87 (m, 3H), 2.84-2.81 (m, 2H), 2.78 (s, 3H); ESI-MS: (+ve mode) 479.15 (M+H)⁺ (100%); HPLC: 99.12%.

Compound 35: N—((S)-2-((S)-3-hydroxypyrrolidin-1-yl)-1-phenylethyl)-2-((8-(2-methoxy-acetamido)quinolin-5-yl)oxy)-N-methylacetamide

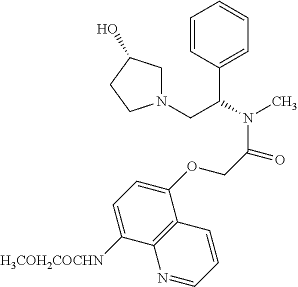

¹H NMR: (DMSO-d6, 400 MHz): 8.97 (s, 1H), 8.67 (d, 1H, J=8.4 Hz), 8.55 (m, 1H), 7.88-7.83 (m, 2H), 7.40-7.33 (m, 3H), 7.24-7.20 (m, 2H), 6.83-6.81 (m, 1H), 6.14-6.11 (m, 2H), 5.45-5.42 (m, 2H), 5.11-5.09 (m, 2H), 4.52-4.44 (m, 2H), 4.19-4.16 (m, 2H), 3.79-3.74 (m, 2H), 3.57-3.54 (m, 1H), 3.52 (s, 3H), 2.89-2.87 (m, 1H), 2.84-2.81 (m, 2H), 2.83 (s, 3H); ESI-MS: (+ve mode) 493.25 (M+H)⁺ (100%); HPLC: 98.82%.

### Compound 36: N—((S)-2-((S)-3-hydroxypyrrolidin-1-yl)-1-phenylethyl)-N-methyl-2-((8-(methyl-sulfonamido)-1,2,3,4-tetrahydroquinolin-5-yl)oxy)acetamide

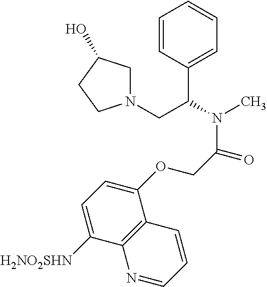

¹H NMR: (DMSO-d6, 400 MHz): 8.95 (s, 1H), 8.66 (d, 1H, J=8.6 Hz), 8.57 (d, 1H, J=8.4 Hz), 7.68-7.61 (m, 2H), 7.52-7.42 (m, 3H), 7.33-7.24 (m, 3H), 6.15-6.12 (m, 2H), 5.45-5.42 (m, 2H), 5.11-5.09 (m, 2H), 4.50-4.42 (m, 2H), 4.18-4.15 (m, 2H), 3.81-3.78 (m, 2H), 2.89-2.87 (m, 3H), 3.12 (s, 2H), 2.52 (s, 1H); ESI-MS: (+ve mode) 500.30 (M+H)⁺ (100%); HPLC: 98.78%.

### Compound 37: N—((S)-2-((S)-3-hydroxypyrrolidin-1-yl)-1-phenylethyl)-N-methyl-2-((8-sulfonamido)-1,2,3,4-tetrahydroquinolin-5-yloxy)acetamide

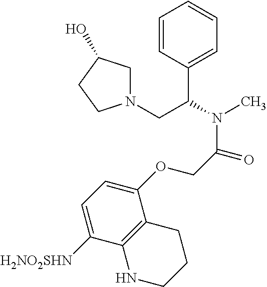

¹H NMR: (DMSO-d6, 400 MHz): 8.95 (s, 1H), 7.68-7.61 (m, 2H), 7.52-7.42 (m, 3H), 7.33-7.24 (m, 2H), 6.15-6.12 (m, 2H), 5.45-5.42 (m, 2H), 5.11-5.09 (m, 2H), 4.50-4.42 (m, 3H), 4.18-4.15 (m, 4H), 3.81-3.78 (m, 4H), 2.84 (s, 3H), 3.12 (s, 2H), 2.52 (s, 2H); ESI-MS: (+ve mode) 504.15 (M+H)⁺ (100%); HPLC: 95.29%.

### Compound 38: N—((S)-2-((S)-3-hydroxypyrrolidin-1-yl)-1-phenylethyl)-N-methyl-2-((8-(pyrrolidine-1-sulfonamido)quinolin-5-yloxy)acetamide

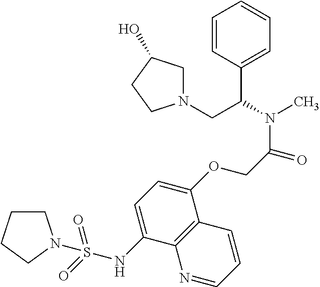

¹H NMR: (DMSO-d6, 400 MHz): 9.04-8.97 (m, 2H), 8.64 (d, 1H, J=9.6 Hz), 7.67-7.60 (m, 2H), 7.42-7.33 (m, 4H), 7.30-7.26 (m, 2H), 6.14-6.11 (m, 1H), 5.35-5.31 (m, 1H), 5.24-5.20 (m, 2H), 4.47-4.42 (m, 2H), 3.71-3.63 (m, 3H), 3.69-3.63 (m, 2H), 3.53-3.50 (m, 2H), 3.19 (m, 6H), 2.85 (s, 3H), 2.33-2.28 (m, 2H); ESI-MS: (+ve mode) 554.25 (M+H)⁺ (100%); HPLC: 96.33%.

### Compound 39: N—((S)-2-((S)-3-hydroxypyrrolidin-1-yl)-1-phenylethyl)-N-methyl-2-((8-(pyrrolidine-1-sulfonamido)-1,2,3,4-tetrahydroquinolin-5-yloxy)acetamide

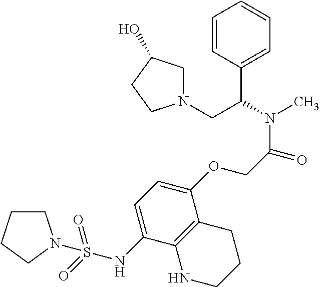

¹H NMR: (DMSO-d6, 400 MHz): 8.98-8.95 (m, 2H), 8.64 (d, 1H, J=9.6 Hz), 7.67-7.60 (m, 2H), 7.42-7.33 (m, 2H), 7.30-7.26 (m, 1H), 6.14-6.11 (m, 1H), 5.35-5.31 (m, 1H), 5.24-5.20 (m, 2H), 4.47-4.42 (m, 2H), 3.71-3.63 (m, 3H), 3.69-3.63 (m, 4H), 3.53-3.50 (m, 4H), 3.19 (m, 6H), 2.85 (s, 3H), 2.33-2.28 (m, 4H); ESI-MS: (+ve mode) 554.25 (M+H)⁺ (100%); HPLC: 96.33%.

### Compound 40: 2-((8-guanidinoquinolin-5-yl)oxy)-N—((S)-2-((S)-3-hydroxypyrrolidin-1-yl)-1-phenylethyl)-N-methylacetamide

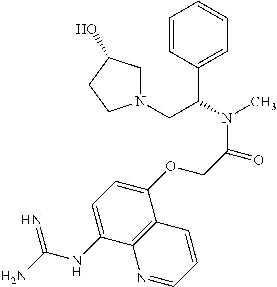

¹H NMR: (DMSO-d6, 400 MHz): 9.68 (d, 1H, J=10.2 Hz), 8.99 (d, 1H, J=8.8 Hz), 8.68 (d, 1H, J=8.6 Hz), 7.67-7.58 (m, 2H), 7.56-7.41 (m, 4H), 7.30-7.24 (m, 2H), 6.16-6.12 (m, 1H), 5.38-5.29 (m, 1H), 4.45-4.41 (m, 2H), 4.23-4.19 (m, 2H), 4.20-4.12 (m, 3H), 3.73-3.69 (m, 2H), 2.87 (d, 3H), 2.33-2.28 (m, 2H); ESI-MS: (+ve mode) 463.05 (M+H)⁺ (100%); HPLC: 96.23%.

### Compound 41: 2-((8-(2-hydroxyethyl)amino)quinolin-5-yl)oxy)-N—((S)-2-((S)-3-hydroxy-pyrrolidin-1-yl)-1-phenylethyl)-N-methylacetamide

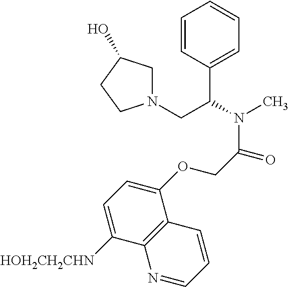

¹H NMR: (DMSO-d6, 400 MHz): 8.96 (s, 1H), 8.66 (s, 1H), 7.88-7.83 (m, 2H), 7.40-7.33 (m, 3H), 7.24-7.20 (m, 2H), 6.83-6.81 (m, 2H), 6.15-6.12 (m, 1H), 5.45-5.42 (m, 1H), 5.11-5.09 (m, 2H), 4.52-4.44 (m, 2H), 4.19-4.16 (m, 2H), 3.79-3.74 (m, 3H), 3.57-3.54 (m, 4H), 2.89-2.87 (m, 3H), 2.84-2.81 (m, 2H), 2.78 (s, 1H); ESI-MS: (+ve mode) 465.15 (M+H)⁺ (100%); HPLC: 99.23%.

### Compound 42: N—((S)-2-((S)-3-hydroxypyrrolidin-1-yl)-1-(3-nitrophenyl)ethyl)-N-methyl-2-((8-(methylsulfonamido)quinolin-5-yl)oxy) acetamide

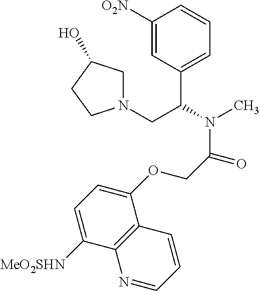

¹H NMR: (DMSO-d6, 400 MHz): 9.16 (d, 1H, J=7.4 Hz), 8.98 (d, 1H, J=7.6 Hz), 8.67-8.65 (m, 1H), 8.23-8.21 (m, 1H), 8.12-8.08 (m, 1H), 7.76-7.69 (m, 2H), 7.66-7.63 (m, 1H), 7.59-7.57 (m, 1H), 7.30-7.27 (m, 2H), 6.25-6.22 (m, 1H), 5.37-5.33 (m, 1H), 5.27-5.23 (m, 2H), 4.29-4.25 (m, 1H), 4.21-4.16 (m, 2H), 3.55-3.49 (m, 2H), 3.24-3.20 (m, 2H), 3.00 (s, 3H), 2.91 (s, 3H), 2.34-2.31 (m, 2H); ESI-MS: (+ve mode) 544.20 (M+H)⁺ (100%); HPLC: 98.23%.

Compound 43: N—((S)-1-(3-aminophenyl)-2-((S)-3-hydroxypyrrolidin-1-yl)ethyl)-N-methyl-2-((8-(methylsulfonamido)quinolin-5-yl)oxy) acetamide

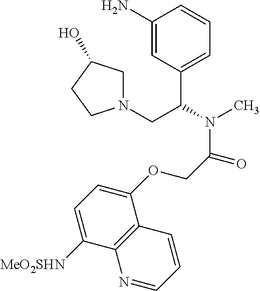

¹H NMR: (DMSO-d6, 400 MHz): 9.14 (d, 1H, J=7.4 Hz), 8.94 (d, 1H, J=7.6 Hz), 8.67-8.65 (m, 1H), 8.23-8.21 (m, 1H), 8.14-8.10 (m, 1H), 7.76-7.69 (m, 2H), 7.66-7.63 (m, 1H), 7.59-7.57 (m, 1H), 7.30-7.27 (m, 2H), 6.26-6.23 (m, 1H), 5.37-5.33 (m, 1H), 5.27-5.23 (m, 2H), 4.29-4.25 (m, 1H), 4.21-4.16 (m, 2H), 3.55-3.49 (m, 2H), 3.24-3.20 (m, 2H), 3.08 (s, 3H), 2.92 (s, 3H), 2.34-2.31 (m, 2H); ESI-MS: (+ve mode) 514.12 (M+H)⁺ (100%); HPLC: 95.45%.

### Compound 44: N—((S)-2-((S)-3-hydroxypyrrolidin-1-yl)-1-(methylsulfonamido)phenyl)ethyl)-N-methyl-2-((8-(methylsulfonamido)quinolin-5-yl)oxy)acetamide

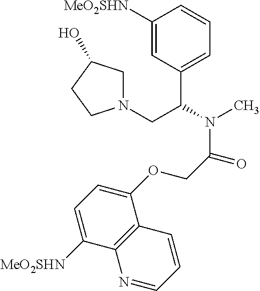

¹H NMR: (DMSO-d6, 400 MHz): 9.85 (d, 1H, J=8.8 Hz), 9.19 (d, 1H, J=7.2 Hz), 8.98-8.97 (m, 1H), 8.67-8.64 (m, 1H), 7.66-7.57 (m, 2H), 7.37-7.26 (m, 2H), 7.26-7.19 (m, 2H), 7.12-7.08 (m, 1H), 6.26-6.23 (m, 1H), 5.37-5.33 (m, 1H), 5.24-5.16 (m, 2H), 4.29-4.25 (m, 1H), 4.21-4.16 (m, 2H), 3.55-3.49 (m, 2H), 3.24-3.20 (m, 2H), 2.99 (s, 6H), 2.92 (s, 3H), 2.34-2.31 (m, 2H); ESI-MS: (+ve mode) 592.15 (M+H)⁺ (100%); HPLC: 98.72%.

### Compound 45: N—((S)-1-(3-acetamidophenyl)-2-((S)-3-hydroxypyrrolidin-1-yl)ethyl)-N-methyl-2-((8-(methylsulfonamido)quinolin-5-yl)oxy) acetamide

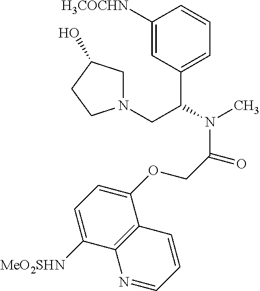

¹H NMR: (DMSO-d6, 400 MHz): 9.18 (d, 1H, J=8.8 Hz), 9.06 (d, 1H, J=7.2 Hz), 8.98-8.97 (m, 1H), 8.68-8.63 (m, 1H), 7.66-7.57 (m, 2H), 7.37-7.26 (m, 2H), 7.26-7.19 (m, 2H), 7.12-7.08 (m, 1H), 6.28-6.24 (m, 1H), 5.38-5.34 (m, 1H), 5.26-5.18 (m, 2H), 4.29-4.25 (m, 1H), 4.21-4.16 (m, 2H), 3.55-3.49 (m, 2H), 3.24-3.20 (m, 2H), 3.00 (s, 6H), 2.85 (s, 3H), 2.34-2.31 (m, 2H); ESI-MS: (+ve mode) 556.15 (M+H)⁺ (100%); HPLC: 95.72%.

### Compound 46: N—((S)-2-((S)-3-hydroxypyrrolidin-1-yl)-1-(methylsulfonamido)phenyl)ethyl)-N-methyl-2-(quinolin-5-yl)oxy) acetamide

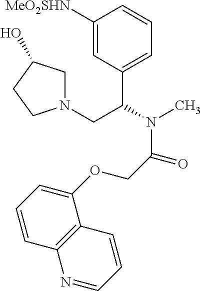

¹H NMR: (DMSO-d6, 400 MHz): 9.82 (d, 1H, J=8.8 Hz), 8.96-8.94 (m, 1H), 8.67-8.64 (m, 1H), 7.66-7.57 (m, 2H), 7.38-7.29 (m, 2H), 7.26-7.19 (m, 2H), 7.12-7.08 (m, 2H), 6.26-6.23 (m, 1H), 5.39-5.35 (m, 1H), 5.24-5.16 (m, 2H), 4.29-4.25 (m, 1H), 4.21-4.16 (m, 2H), 3.55-3.49 (m, 2H), 3.24-3.20 (m, 2H), 2.96 (s, 3H), 2.85 (s, 3H), 2.36-2.34 (m, 2H); ESI-MS: (+ve mode) 499.05 (M+H)⁺ (100%); HPLC: 99.34%.

### Compound 47: N-((8)-1-(3-dimethylaminophenyl)-2-((S)-3-hydroxypyrrolidin-1-yl)ethyl)-N-methyl-2-((8-(methylsulfonamido)quinolin-5-yl)oxy) acetamide

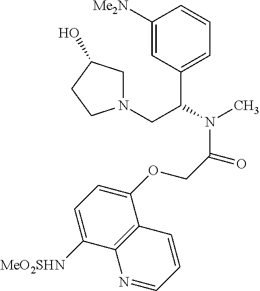

¹H NMR: (DMSO-d6, 400 MHz): 9.16 (d, 1H, J=7.2 Hz), 8.88-8.86 (m, 1H), 8.66-8.63 (m, 1H), 7.68-7.59 (m, 2H), 7.37-7.26 (m, 2H), 7.26-7.19 (m, 2H), 7.16-7.12 (m, 1H), 6.26-6.23 (m, 1H), 5.37-5.33 (m, 1H), 5.24-5.16 (m, 2H), 4.29-4.25 (m, 1H), 4.21-4.16 (m, 2H), 3.55-3.49 (m, 2H), 3.24-3.20 (m, 2H), 2.92 (s, 6H), 2.55 (s, 6H), 2.34-2.31 (m, 2H); ESI-MS: (+ve mode) 542.25 (M+H)⁺ (100%); HPLC: 99.16%.

Compound 48: N—((S)-1-(3-hydroxyphenyl)-2-((S)-3-hydroxypyrrolidin-1-yl)ethyl)-N-methyl-2-((8-(methylsulfonamido)quinolin-5-yl)oxy) acetamide

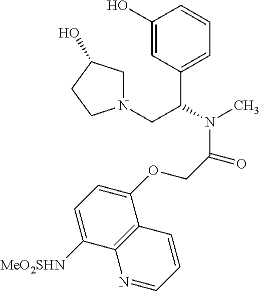

¹H NMR: (DMSO-d6, 400 MHz): 9.16 (d, 1H, J=7.6 Hz), 8.92 (d, 1H, J=7.4 Hz), 8.68-8.67 (m, 1H), 8.23-8.21 (m, 1H), 8.14-8.10 (m, 1H), 7.76-7.69 (m, 2H), 7.66-7.63 (m, 1H), 7.59-7.57 (m, 1H), 7.30-7.27 (m, 2H), 6.26-6.23 (m, 1H), 5.37-5.33 (m, 1H), 5.27-5.23 (m, 2H), 4.29-4.25 (m, 1H), 4.21-4.16 (m, 2H), 3.55-3.49 (m, 2H), 3.24-3.20 (m, 2H), 3.00 (s, 3H), 2.95 (s, 3H), 2.34-2.31 (m, 2H); ESI-MS: (+ve mode) 514.45 (M+H)⁺ (100%); HPLC: 97.84%.

### Compound 49: N—((S)-2-((S)-3-hydroxypyrrolidin-1-yl)-1-(3-methoxyphenyl)ethyl)-N-methyl-2-((8-(methylsulfonamido)quinolin-5-yl)oxy) acetamide

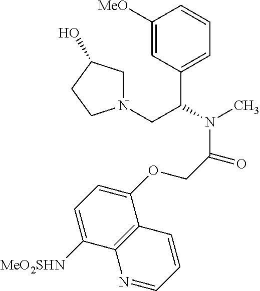

¹H NMR: (DMSO-d6, 400 MHz): 9.22 (d, 1H, J=7.6 Hz), 8.89 (d, 1H, J=7.4 Hz), 8.67-8.65 (m, 1H), 8.23-8.21 (m, 1H), 8.16-8.12 (m, 1H), 7.76-7.69 (m, 2H), 7.68-7.64 (m, 1H), 7.59-7.57 (m, 1H), 7.30-7.27 (m, 1H), 6.26-6.23 (m, 1H), 5.37-5.33 (m, 1H), 5.27-5.23 (m, 2H), 4.29-4.25 (m, 1H), 4.21-4.16 (m, 2H), 4.14 (s, 3H), 3.55-3.49 (m, 2H), 3.24-3.20 (m, 2H), 2.99 (s, 3H), 2.85 (s, 3H), 2.34-2.31 (m, 2H); ESI-MS: (+ve mode) 529.15 (M+H)⁺ (100%); HPLC: 96.83%.

Compound 50: Ethyl-2-(3((S)-2-((S)-3-hydroxypyrrolidin-1-yl)-1-N-methyl-2-((8-(methyl-sulfonamido)quinolin-5-yl)oxy)acetamido)ethyl) phenoxy)acetate

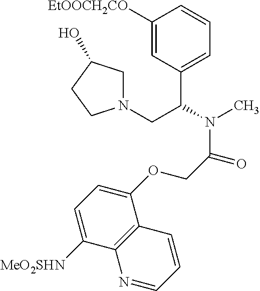

¹H NMR: (DMSO-d6, 400 MHz): 9.00 (d, 1H, J=8.8 Hz), 8.96 (d, 1H, J=7.2 Hz), 7.83-7.79 (m, 2H), 7.79-7.77 (m, 2H), 7.63-7.60 (m, 1H), 7.20-7.17 (m, 1H), 6.76-6.69 (m, 2H), 6.11-6.08 (m, 1H), 5.37-5.33 (m, 1H), 5.24-5.16 (m, 2H), 4.29-4.25 (m, 3H), 4.21-4.16 (m, 2H), 4.08-4.05 (m, 2H), 3.55-3.49 (m, 4H), 3.24-3.20 (m, 2H), 3.14 (s, 3H), 2.85 (s, 3H), 2.34-2.31 (m, 2H); ESI-MS: (+ve mode) 601.35 (M+H)⁺ (100%); HPLC: 98.19%.

Compound 51: 2-(3((S)-2-((S)-3-hydroxypyrrolidin-1-yl)-1-N-methyl-2-((8-(methyl sulfonamido) quinolin-5-yl)oxy)acetamido)ethyl) phenoxy)acetic acid

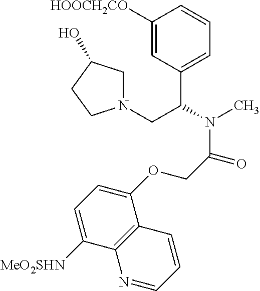

¹H NMR: (DMSO-d6, 400 MHz): 9.00 (d, 1H, J=8.8 Hz), 8.98 (d, 1H, J=7.2 Hz), 7.83-7.79 (m, 2H), 7.79-7.77 (m, 2H), 7.64-7.61 (m, 1H), 7.22-7.19 (m, 1H), 6.78-6.70 (m, 2H), 6.11-6.08 (m, 1H), 5.37-5.33 (m, 1H), 5.24-5.16 (m, 2H), 4.29-4.25 (m, 3H), 4.21-4.16 (m, 2H), 3.55-3.49 (m, 4H), 3.24-3.20 (m, 2H), 2.86 (s, 3H), 2.36-2.32 (m, 2H); ESI-MS: (+ve mode) 573.25 (M+H)⁺ (100%); HPLC: 96.90%.

### Compound 52: N—((S)-1-(3-fluorophenyl)-2-((S)-3-hydroxypyrrolidin-1-yl)ethyl)-N-methyl-2-((8-(methylsulfonamido)quinolin-5-yl)oxy) acetamide

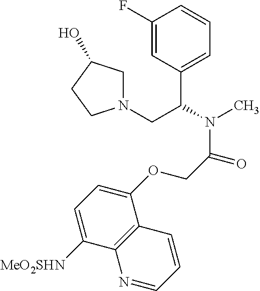

¹H NMR: (DMSO-d6, 400 MHz): 9.22 (d, 1H, J=7.4 Hz), 8.86 (d, 1H, J=7.6 Hz), 8.76-8.75 (m, 1H), 8.23-8.21 (m, 1H), 8.15-8.11 (m, 1H), 7.78-7.67 (m, 2H), 7.66-7.63 (m, 1H), 7.59-7.57 (m, 1H), 7.30-7.27 (m, 1H), 6.25-6.22 (m, 1H), 5.37-5.33 (m, 1H), 5.27-5.23 (m, 2H), 4.29-4.25 (m, 1H), 4.24-4.19 (m, 2H), 3.55-3.49 (m, 2H), 3.24-3.20 (m, 2H), 3.12 (s, 3H), 2.96 (s, 3H), 2.34-2.31 (m, 2H); ESI-MS: (+ve mode) 517.30 (M+H)⁺ (100%); HPLC: 99.12%.

### Compound 53: N—((S)-2-((S)-3-hydroxypyrrolidin-1-yl)-1-(3-(trifluoromethyl)phenyl)ethyl)-N-methyl-2-((8-(methylsulfonamido)quinolin-5-yl)oxy) acetamide

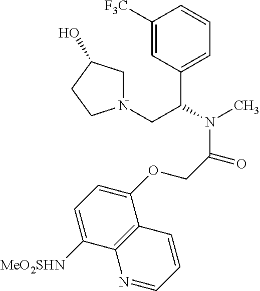

¹H NMR: (DMSO-d6, 400 MHz): 9.19 (d, 1H, J=7.6 Hz), 8.84 (d, 1H, J=7.8 Hz), 8.78-8.77 (m, 1H), 8.25-8.22 (m, 1H), 8.15-8.11 (m, 1H), 7.78-7.67 (m, 2H), 7.66-7.63 (m, 1H), 7.59-7.57 (m, 1H), 7.30-7.27 (m, 1H), 6.25-6.22 (m, 1H), 5.39-5.32 (m, 1H), 5.28-5.24 (m, 2H), 4.29-4.25 (m, 1H), 4.24-4.19 (m, 2H), 3.55-3.49 (m, 2H), 3.24-3.20 (m, 2H), 3.10 (s, 3H), 2.92 (s, 3H), 2.34-2.31 (m, 2H); ESI-MS: (+ve mode) 567.35 (M+H)⁺ (100%); HPLC: 95.14%.

### Compound 54: N—((S)-2-((S)-3-hydroxypyrrolidin-1-yl)-1-(3-methyl)ethyl)-N-methyl-2-((8-(methylsulfonamido)quinolin-5-yl)oxy)acetamide

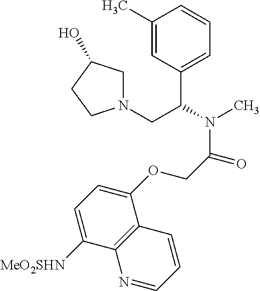

¹H NMR: (DMSO-d6, 400 MHz): 9.23 (d, 1H, J=7.6 Hz), 8.94 (d, 1H, J=7.8 Hz), 8.86-8.85 (m, 1H), 8.35-8.32 (m, 1H), 8.18-8.15 (m, 1H), 7.80-7.66 (m, 2H), 7.66-7.63 (m, 1H), 7.59-7.57 (m, 1H), 7.30-7.27 (m, 1H), 6.25-6.22 (m, 1H), 5.39-5.32 (m, 1H), 5.29-5.26 (m, 2H), 4.29-4.25 (m, 1H), 4.24-4.19 (m, 2H), 3.55-3.49 (m, 2H), 3.24-3.20 (m, 2H), 3.12 (s, 3H), 2.96 (s, 3H), 2.34-2.31 (m, 2H); ESI-MS: (+ve mode) 513.15 (M+H)⁺ (100%); HPLC: 98.36%.

### Compound 55: 2-(benzofuran-6-yloxy)-N—((S)-2-((S)-3-hydroxypyrrolidin-1-yl)-1-(3-nitro-phenyl)ethyl)-N-methylacetamide

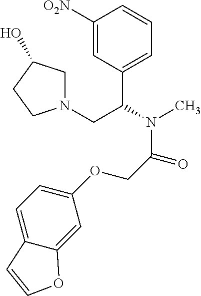

¹H NMR: (DMSO-d6, 400 MHz): 8.16 (d, 1H, J=8.2 Hz), 8.08 (d, 1H, J=6.8 Hz), 7.86-7.84 (m, 1H), 7.52-7.48 (m, 2H), 7.39-7.34 (m, 2H), 6.98-6.95 (m, 1H), 6.86-6.85 (m, 1H), 6.23-6.20 (m, 1H), 5.14-5.07 (m, 2H), 4.46-4.42 (m, 2H), 4.19-4.06 (m, 2H), 3.88-3.83 (m, 4H), 3.38-3.35 (m, 2H), 2.85 (d, 3H), 1.91-1.89 (m, 1H); ESI-MS: (+ve mode) 439.95 (M+H)⁺ (100%); HPLC: 97.93%.

### Compound 56: 2-(benzofuran-6-yloxy)-N—((S)-2-((S)-3-hydroxypyrrolidin-1-yl)-1-(3-(methyl-sulfonamido)phenyl)ethyl)-N-methylacetamide

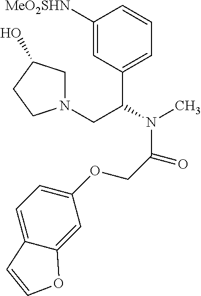

¹H NMR: (DMSO-d6, 400 MHz): 8.19 (d, 1H, J=8.4 Hz), 8.06 (d, 1H, J=6.8 Hz), 7.89-7.86 (m, 1H), 7.54-7.49 (m, 2H), 7.40-7.37 (m, 2H), 6.98-6.95 (m, 1H), 6.86-6.85 (m, 1H), 6.23-6.20 (m, 1H), 5.14-5.07 (m, 2H), 4.46-4.42 (m, 2H), 4.20-4.09 (m, 2H), 3.89-3.86 (m, 4H), 3.38-3.35 (m, 2H), 3.10 (s, 3H), 2.85 (d, 3H), 1.91-1.89 (m, 1H); ESI-MS: (+ve mode) 488.25 (M+H)⁺ (100%); HPLC: 98.53%.

### Compound 57: 2-(benzo[d][1,3]dioxol-5-yloxy)-N—((S)-2-((S)-3-hydroxypyrrolidin-1-yl)-1-(3-(methylsulfonamido)phenyl)ethyl)-N-methylacetamide

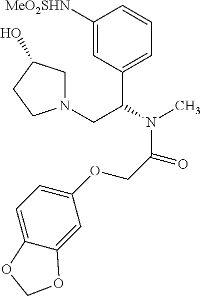

¹H NMR: (DMSO-d6, 400 MHz): 7.36-7.34 (m, 1H), 7.33-7.31 (m, 1H), 7.21-7.17 (m, 1H), 7.07-7.01 (m, 1H), 6.78-6.75 (m, 2H), 6.44-6.41 (m, 1H), 5.95 (s, 2H), 5.56-5.52 (m, 1H), 4.86-4.84 (m, 3H), 4.43-4.40 (m, 2H), 3.63-3.59 (m, 2H), 2.98 (s, 3H), 2.75 (d, 3H), 1.54-1.52 (m, 1H); ESI-MS: (+ve mode) 493.00 (M+H)⁺ (100%); HPLC: 96.75%.

### Compound 58: Methyl-(3-((S)-1-(2-(benzo[d][1,3]dioxol-5-yloxy)-N-methylacetamido)-2-((S)-3-hydroxypyrrolidin-1-yl)-ethyl)phenyl)carbamate

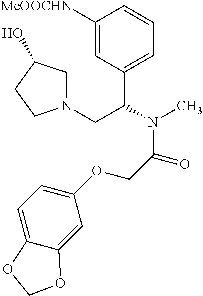

¹H NMR: (DMSO-d6, 400 MHz): 7.40-7.37 (m, 1H), 7.36-7.34 (m, 1H), 7.24-7.19 (m, 1H), 7.07-7.01 (m, 1H), 6.79-6.77 (m, 2H), 6.44-6.41 (m, 1H), 5.95 (s, 2H), 5.56-5.52 (m, 1H), 4.86-4.84 (m, 3H), 4.43-4.40 (m, 2H), 3.63-3.59 (m, 2H), 3.48-3.45 (m, 3H), 3.10 (s, 3H), 2.75 (d, 3H), 1.54-1.52 (m, 2H); ESI-MS: (+ve mode) 472.25 (M+H)⁺ (100%); HPLC: 98.24%.

Using the above procedure, following compounds listed in Table-2 can be prepared.

TABLE 2

| S. No | Structures | IUPAC Names |
|---|---|---|
| 59 | 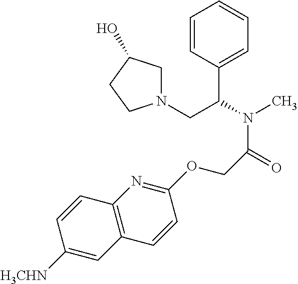 | N-((S)-2-((S)-3-hydroxypyrrolidin-1-yl)-1-phenylethyl)-N- methyl-2-((6-methylamino)quinolin-2-yloxy)acetamide |
| 60 | 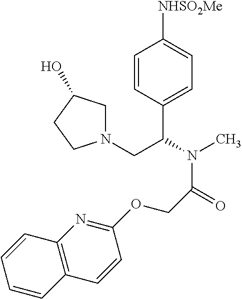 | N-((S)-2-((S)-3-hydroxypyrrolidin-1-yl)-1-(4- (methylsulfonamido)phenyl)ethyl)-N-methyl-2-(quinolin-2- yloxy)acetamide |
| 61 | 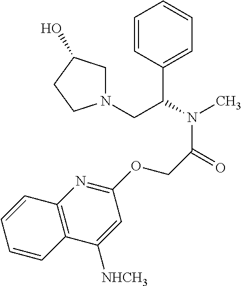 | N-((S)-2-((S)-3-hydroxypyrrolidin-1-yl)-1-phenylethyl)-N- methyl-2-((4-methylamino)quinolin-2-yloxy)acetamide |
| 62 | 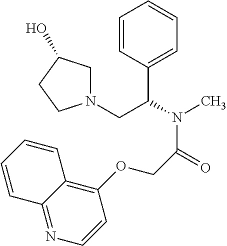 | N-((S)-2-((S)-3-hydroxypyrrolidin-1-yl)-1-phenylethyl)-N- methyl-2-(quinolin-4-yloxy)acetamide |
| 63 | 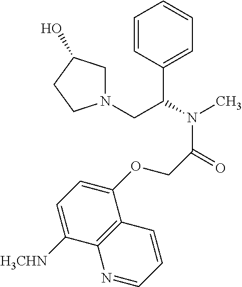 | N-((S)-2-((S)-3-hydroxypyrrolidin-1-yl)-1-phenylethyl)-N- methyl-2-((8-methylamino)quinolin-5-yloxy)acetamide |
| 64 | 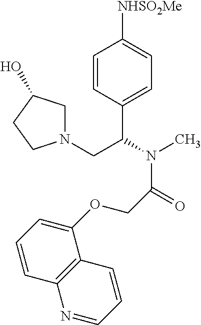 | N-((S)-2-((S)-3-hydroxypyrrolidin-1-yl)-1-(4- (methylsulfonamido)phenyl)ethyl)-N-methyl-2-(quinolin-5- yloxy)acetamide |
| 65 | 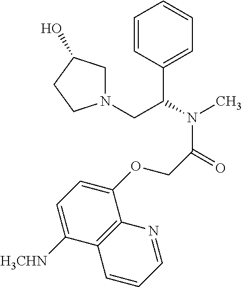 | N-((S)-2-((S)-3-hydroxypyrrolidin-1-yl)-1-phenylethyl)-N- methyl-2-((5-methylamino)quinolin-8-yloxy)acetamide |
| 66 | 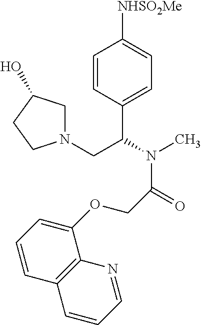 | N-((S)-2-((S)-3-hydroxypyrrolidin-1-yl)-1-(4- (methylsulfonamido)phenyl)ethyl)-N-methyl-2-(quinolin-8- yloxy)acetamide |
| 67 | 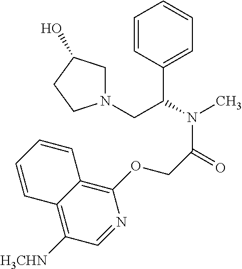 | N-((S)-2-((S)-3-hydroxypyrrolidin-1-yl)-1-phenylethyl)-N- methyl-2-((4-methylamino)isoquinolin-1-yloxy)acetamide |
| 68 | 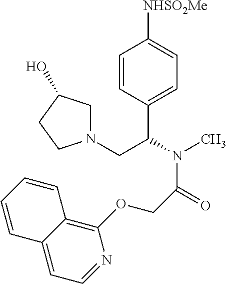 | N-((S)-2-((S)-3-hydroxypyrrolidin-1-yl)-1-(4- (methylsulfonamido)phenyl)ethyl)2-(isoquinolin-1-yloxy)- N-methylacetamide |
| 69 | 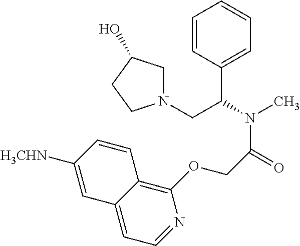 | N-((S)-2-((S)-3-hydroxypyrrolidin-1-yl)-1-phenylethyl)-N- methyl-2-((6-methylamino)isoquinolin-1-yloxy)acetamide |
| 70 | 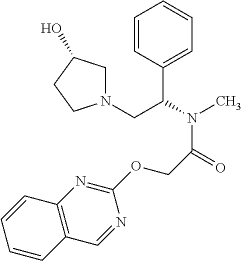 | N-((S)-2-((S)-3-hydroxypyrrolidin-1-yl)-1-phenylethyl)-N- methyl-2-(quinazolin-2-yloxy)acetamide |
| 71 | 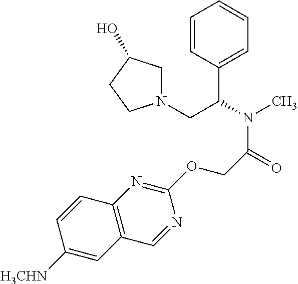 | N-((S)-2-((S)-3-hydroxypyrrolidin-1-yl)-1-phenylethyl)-N- methyl-2-((6-methylamino)quinazolin-2-yloxy)acetamide |
| 72 | 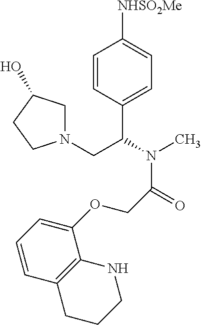 | N-((S)-2-((S)-3-hydroxypyrrolidin-1-yl)-(4- (methylsulfonamido)phenyl)ethyl)-N-methyl-2-(1,2,3,4- tetrahydroquinolin-8-yloxy)acetamide |
| 73 | 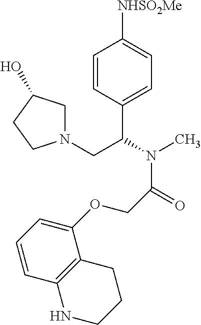 | N-((S)-2-((S)-3-hydroxypyrrolidin-1-yl)-(4- (methylsulfonamido)phenyl)ethyl)-N-methyl-2-(1,2,3,4- tetrahydroquinolin-5-yloxy)acetamide |
| 74 | 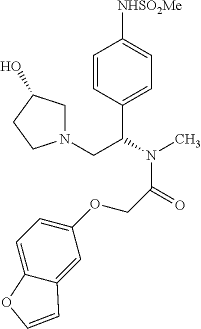 | 2-((benzofuran-5-yloxy)-N-((S)-2-((S)-3-hydroxy- pyrrolidin-1-yl)-1-(4-(methylsulfonamido)phenyl)ethyl)-N- methylacetamide |
| 75 | 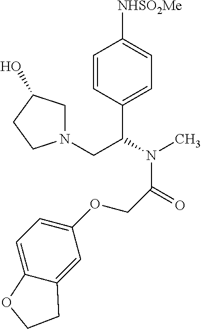 | 2-(((2,3-dihydrobenzofuran-5-yloxy)-N-((S)-2-((S)-3- hydroxy-pyrrolidin-1-yl)-1-(4- (methylsulfonamido)phenyl)ethyl)-N-methylacetamide |
| 76 | 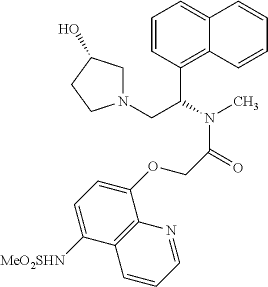 | N-((S)-2-((S)-3-hydroxypyrrolidin-1-yl)-1-(naphthalene-1- yl)ethyl)-N-methyl-2-((5-((methylsulfonamido)-quinolin-8- yl)oxy)acetamide |
| 77 | 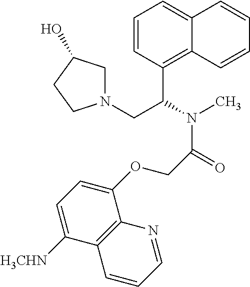 | N-((S)-2-((S)-3-hydroxypyrrolidin-1-yl)-1-(naphthalene-1- yl)ethyl)-N-methyl-2-((5-((methylamino)-quinolin-8- yl)oxy)acetamide |
| 78 | 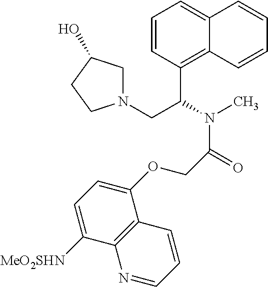 | N-((S)-2-((S)-3-hydroxypyrrolidin-1-yl)-1-(naphthalene-1- yl)ethyl)-N-methyl-2-((8-((methylsulfonamido)-quinolin-5- yl)oxy)acetamide |
| 79 | 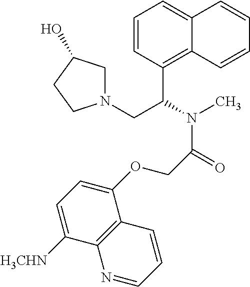 | N-((S)-2-((S)-3-hydroxypyrrolidin-1-yl)-1-(naphthalene-1- yl)ethyl)-N-methyl-2-((8-((methylamino)-quinolin-5- yl)oxy)acetamide |
| 80 | 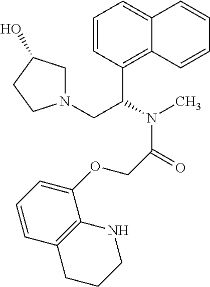 | N-((S)-2-((S)-3-hydroxypyrrolidin-1-yl)-1-(naphthalene-1- yl)ethyl)-N-methyl-2-((1,2,3,4-tetrahydroquinolin-8- yl)oxy)acetamide |
| 81 | 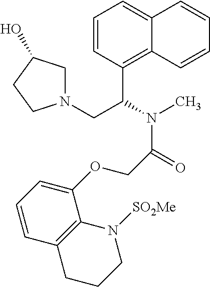 | N-((S)-2-((S)-3-hydroxypyrrolidin-1-yl)-1-(naphthalene-1- yl)ethyl)-N-methyl-2-((1-(methylsulfonyl)-1,2,3,4- tetrahydroquinolin-8-yl)oxy)acetamide |
| 82 | 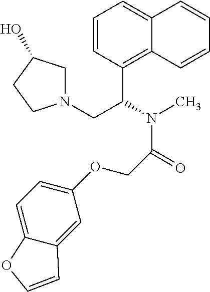 | 2-(benzofuran-5-yloxy)-N-((S)-2-((S)-3-hydroxypyrrolidin- 1-yl)-1-(naphthalene-1-yl)ethyl)-N-methylacetamide |
| 83 | 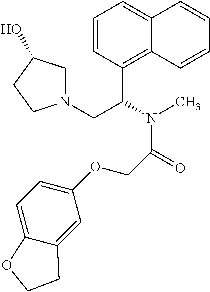 | 2-42,3-dihydrobenzofuran-5-yloxy)-N-((S)-2-((S)-3- hydroxypyrrolidin-1-yl)-1-(naphthalene-1-yl)ethyl)-N- methylacetamide |
| 84 | 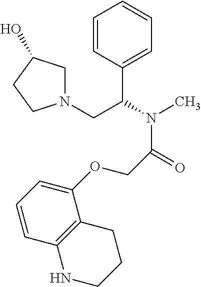 | N-((S)-2-((S)-3-hydroxypyrrolidin-1-yl)-1-phenylethyl)-N- methyl-2-((1,2,3,4-tetrahydroquinolin-4-yl)oxy)acetamide |
| 85 | 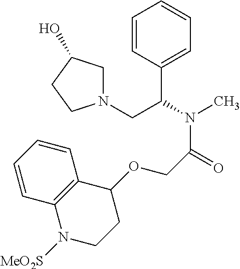 | N-((S)-2-((S)-3-hydroxypyrrolidin-1-yl)-1-phenylethyl)-N- methyl-2-((1-(methylsulfonyl)-1,2,3,4-tetrahydroquinolin- 4-yl)oxy)acetamide |
| 86 | 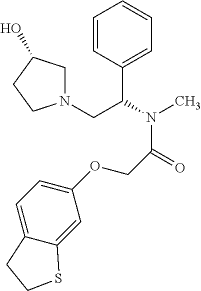 | 2-((2,3-dihydrobenzo[b]thiophen-6-yl)oxy)-N-((S)-2-((S)- 3-hydroxypyrrolidin-1-yl)-1-phenylethyl)-N- methylacetamide |
| 87 | 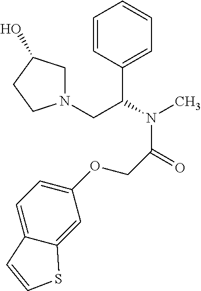 | 2-(benzo[b]thiophen-6-yloxy)-N-((S)-2-((S)-3- hydroxypyrrolidin-1-yl)-1-phenylethyl)-N-methylacetamide |
| 88 | 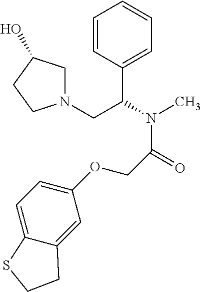 | 2-((2,3-dihydrobenzo[b]thiophen-5-yl)oxy)-N-((S)-2-((S)- 3-hydroxypyrrolidin-1-yl)-1-phenylethyl)-N- methylacetamide |
| 89 | 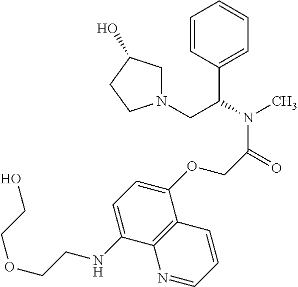 | 2-((8-(2-hydroxyethoxy)ethyl)amino)quinolin-5-yl)oxy)-N- ((S)-2-((S)-3-hydroxypyrrolidin-1-yl)-1-phenylethyl)-N- methylacetamide |
| 90 | 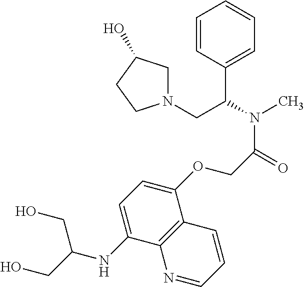 | 2-((8-(1,3-dihydroxypropan-2-yl)amino)quinolin-5- yl)oxy)-N-((S)-2-((S)-3-hydroxypyrrolidin-1-yl)- 1-phenylethyl)-N-methylacetamide |
| 91 | 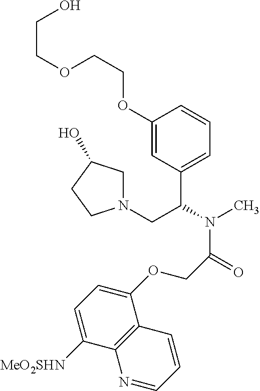 | N-((S)-1-((3-(2-(2-hydroxyethoxy)ethoxyl)phenyl)-2-((S)- 3-hydroxypyrrolidin-1-yl)ethyl)-N-methyl-2-((8- (methylsulfonamido)quinolin-5-yl)oxy)acetamide |
| 92 | 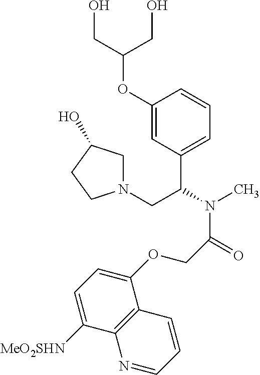 | N-((S)-1-((3-((1,3-dihydroxypropan-2-yl)oxy)phenyl)-2- ((S)-3-hydroxypyrrolidin-1-yl)ethyl)-N-methyl-2-((8- (methylsulfonamido)quinolin-5-yl)oxy)acetamide |
| 93 | 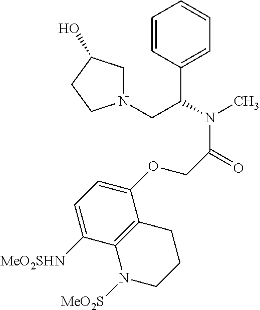 | N-((S)-2-((S)-3-hydroxypyrrolidin-1-yl)-1-phenylethyl)-N- methyl-2-((8-(methylsulfonamido)-1-(methylsulfonyl)- 1,2,3,4-tetrahydroquinolin-5-yl)oxy)acetamide |
| 94 | 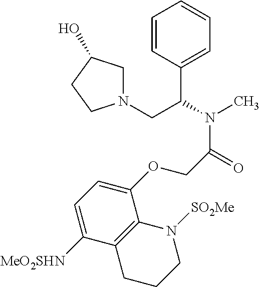 | N-((S)-2-((S)-3-hydroxypyrrolidin-1-yl)-1-phenylethyl)-N- methyl-2-((5-(methylsulfonamido)-1-(methylsulfonyl)- 1,2,3,4-tetrahydroquinolin-8-yl)oxy)acetamide |
| 95 | 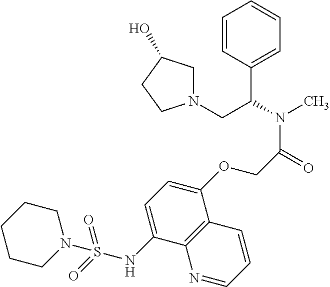 | N-((S)-2-((S)-3-hydroxypyrrolidin-1-yl)-1-phenylethyl)-N- methyl-2-((8-(piperidine-1-sulfonamido)quinolin-5- yl)oxy)acetamide |
| 96 | 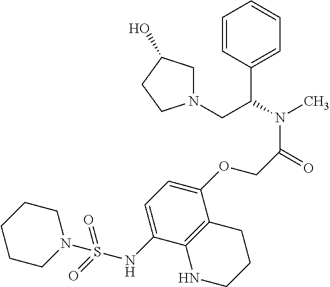 | N-((S)-2-((S)-3-hydroxypyrrolidin-1-yl)-1-phenylethyl)-N- methyl-2-((8-(piperidine-1-sulfonamido)-1,2,3,4- tetrahydroquinolin-5-yl)oxy)acetamide |
| 97 | 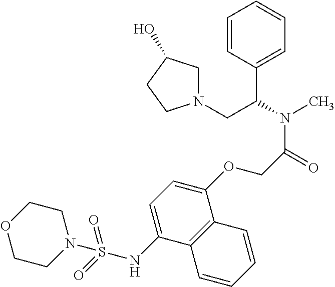 | N-((S)-2-((S)-3-hydroxypyrrolidin-1-yl)-1-phenylethyl)-N- methyl-2-((8-(morpholine-4-sulfonamido)quinolin-5- yl)oxy)acetamide |
| 98 | 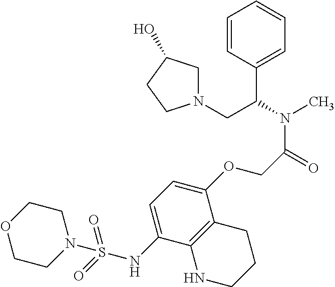 | N-((S)-2-((S)-3-hydroxypyrrolidin-1-yl)-1-phenylethyl)-N- methyl-2-((8-(morpholine-4-sulfonamido)-1,2,3,4- tetrahydroquinolin-5-yl)oxy)acetamide |
| 99 | 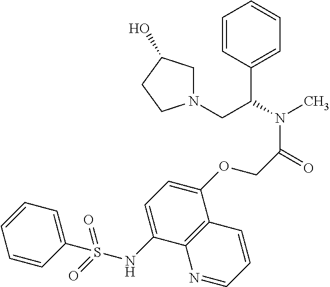 | N-((S)-2-((S)-3-hydroxypyrrolidin-1-yl)-1-phenylethyl)-N- methyl-2-((8-(phenylsulfonamido))quinolin-5- yl)oxy)acetamide |
| 100 | 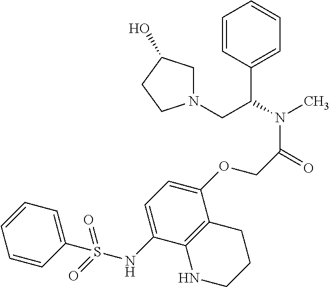 | N-((S)-2-((S)-3-hydroxypyrrolidin-1-yl)-1-phenylethyl)-N- methyl-2-((8-(phenylsufonamido)-1,2,3,4- tetrahydroquinolin-5-yl)oxy)acetamide |
| 101 | 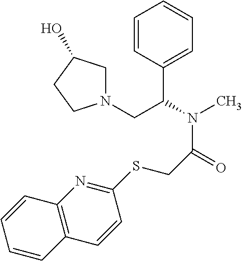 | N-((S)-2-((S)-3-hydroxypyrrolidin-1-yl)-1-phenylethyl)-N- methyl-2-(quinolin-2-ylthio)acetamide |
| 102 | 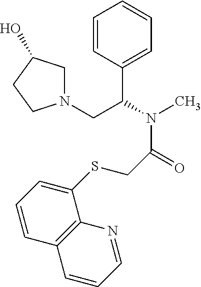 | N-((S)-2-((S)-3-hydroxypyrrolidin-1-yl)-1-phenylethyl)-N- methyl-2-(quinolin-8-ylthio)acetamide |
| 103 | 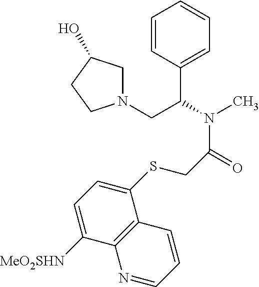 | N-((S)-2-((S)-3-hydroxypyrrolidin-1-yl)-1-phenylethyl)-N- methyl-2-((8-methylsulfonamido)quinolin-5- ylthio)acetamide |
| 104 | 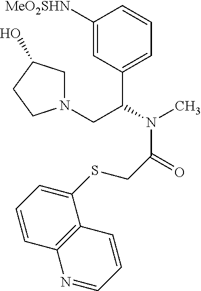 | N-((S)-2-((S)-3-hydroxypyrrolidin-1-yl)-1- (methylsulfonamido)phenyl)ethyl)-N-methyl-2-(quinolin-5- yl)thio)acetamide |
| 105 | 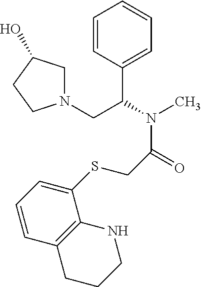 | N-((S)-2-((S)-3-hydroxypyrrolidin-1-yl)-1-phenylethyl)-N- methyl-2-(1,2,3,4-tetrahydroquinolin-8-ylthio)acetamide |
| 106 | 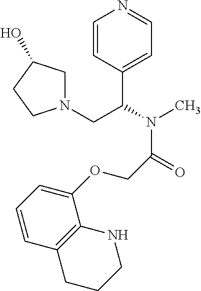 | N-((S)-2-((S)-3-hydroxypyrrolidin-1-yl)-1-(pyridin-4- yl)ethyl)-N-methyl-2-((1,2,3,4-tetrahydroquinolin-8- yl)oxy)acetamide |
| 107 | 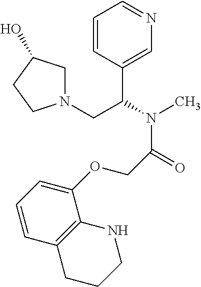 | N-((S)-2-((S)-3-hydroxypyrrolidin-1-yl)-1-(pyridin-3- yl)ethyl)-N-methyl-2-((1,2,3,4-tetrahydroquinolin-8- yl)oxy)acetamide |
| 108 | 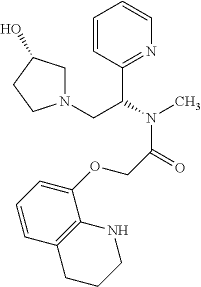 | N-((R)-2-((S)-3-hydroxypyrrolidin-1-yl)-1-(pyridin-2- yl)ethyl)-N-methyl-2-((1,2,3,4-tetrahydroquinolin-8- yl)oxy)acetamide |

### Biological Activity Screening:

a) Ex-Vivo KOR Agonistic Activity Testing, Using Electrically Stimulated Mouse Vas Deferens (MVD) Model:

Ex-vivo, the kappa opioid receptor agonistic activity of test compounds were tested on the electrically stimulated mouse vas deferens (MVD) preparations (Henderson et al., Br. J. Pharmacol., 46, 764-766, 1972; Portoghese et al., Life Sci. 36, 801-805, 1985) and IC₅₀ were determined. In general, vas deferentia were taken from male Swiss Albino mice (30-40 g) and suspended in 8 ml organ baths, at 31° C., containing modified Krebs-Henseleit solution, without magnesium sulphate. Each vas deferens was equilibrated for 45 min at 2.6 mN tension and then stimulated at supramaximal voltage with five 1 ms pulses, at a frequency of 0.1 Hz. Concentration-response curves were determined by cumulative dosing. Inhibitory Concentration, 50% (IC₅₀) values were determined by Sigmoidal dose-response (variable slope) equation, using Prizm v 6.01. The kappa opioid receptor specificity was determined by rightward shift in concentration-response curves in presence of 1 nM norbinaltorphimine (norBNI), a selective kappa opioid receptor antagonist. The ex-vivo kappa opioid receptor agonistic activities (IC₅₀) for representative compounds are listed in Table 3.

b) Invitro (EC₅₀) Determination, Using cAMP Based Functional Assay:

Invitro, KOR agonistic activity of test compounds were assessed using cAMP based functional assay. A 96-well plate was seeded at the density of 30,000 cells/well in 100 μl/well of complete Ham's F-12 medium. After seeding, the plates were incubated overnight at 37° C., 5% CO₂ in CO₂ Incubator. Overnight medium was discarded and plate washed with 100 μl/well of sterile PBS. Then 90 μl of 0.1 mM IBMX containing 0.5% Fatty acid free BSA in plain HamsF12 was added to each well. This was allowed to incubate for 30 minutes at 37° C., 5% CO₂. Forskolin 20 μM in 0.5% Fatty acid free BSA was added to each well and allowed to incubate at room temperature for 5 minutes. Dilution of test compounds was made at 200× in DMSO and then diluted 1:10 times in BSA containing plain HamsF12. Agonist (test compounds, in 10% DMSO) was added to each well (5 μl) and allowed to incubate for 20 minutes at 37° C., 5% CO₂. After 20 minutes, media was aspirated from the wells and the wells were washed with 1×PBS. Cell lysis buffer 4× (Arbor Assays, Cat # X074-60ML) was diluted 1:4 in MilliQ and 90 μl of this buffer was added per well. Cells were allowed to shake at 500 rpm, room temperature for 20 minutes. Cell lysate was collected in 1.5 ml eppendorf tubes and centrifuged at 13.2 k rpm, 4° C. for 15 minutes. 50 μl of the supernatant of cell lysate was then used for cAMP estimation by cAMP direct ELISA kit (Arbor Assays, Cat # K019-H5). The invitro kappa opioid receptor agonistic activities (EC₅₀) for representative compounds are listed in Table 3

TABLE 3
Ex-vivo (IC₅₀) and Invitro (EC₅₀)
data of representative compounds

| Compd No. | Ex-vivo IC₅₀ nM | In-vitro EC₅₀ nM |
|---|---|---|
| 1 | 3.5 | 0.022 |
| 2 | 2590 | 4.93 |
| 3 | 414 | 0.035 |
| 4 | 1500 | 1.21 |
| 5 | 5.6 | 0.013 |
| 6 | 349 | 0.94 |
| 7 | 0.84 | 0.015 |
| 8 | 6.2 | 0.018 |
| 9 | 13 | 0.012 |
| 10 | 54 | 0.089 |
| 11 | 1.6 | 0.004 |
| 12 | 2 | 0.005 |
| 13 | 125 | 0.91 |
| 14 | 5.4 | 0.012 |
| 15 | 0.9 | 0.003 |
| 16 | 4 | 0.040 |
| 17 | 7 | 0.012 |
| 18 | 8.2 | 0.07 |
| 19 | 135 | 1.31 |
| 20 | 1740 | 2.12 |
| 21 | 570 | 1.23 |
| 22 | 8.1 | 0.008 |
| 23 | 8.4 | 0.017 |
| 24 | 5 | 0.055 |
| 25 | 8.1 | 0.003 |
| 26 | 29 | 0.012 |
| 27 | 169 | 2.11 |
| 28 | 1490 | 1.42 |
| 29 | 147 | 0.11 |
| 30 | 9400 | 900 |
| 31 | 118 | 0.51 |
| 32 | 0.49 | 0.013 |
| 33 | 8.4 | 0.006 |
| 34 | 258 | 0.49 |
| 35 | 48 | 0.060 |
| 36 | 7.8 | 1.12 |
| 37 | 0.3 | 0.013 |
| 38 | 7.4 | 0.95 |
| 39 | 0.95 | 0.088 |
| 40 | 1.1 | 0.007 |
| 41 | 254 | 1.32 |
| 42 | 50 | 0.044 |
| 43 | 1.6 | 0.006 |
| 44 | 5.4 | 0.036 |
| 45 | 14 | 0.066 |
| 46 | 9.5 | 0.042 |
| 47 | 88 | 0.691 |
| 48 | 1.3 | 0.021 |
| 49 | 1.9 | 0.032 |
| 50 | 221 | 0.001 |
| 51 | 16 | 0.11 |
| 52 | 21 | 0.18 |
| 53 | 73 | 0.68 |
| 54 | 14 | 0.098 |
| 55 | 307 | 1.92 |
| 56 | 88 | 1.11 |
| 57 | 27 | 0.961 |
| 58 | 9.8 | 0.098 |

### In Vivo Efficacy Studies:

### Animals

All the animal experiments were carried out in ICR mice, bred in-house. Animals were housed in groups of 6 animals per cage, for a week, in order to habituate them to vivarium conditions (25±4° C., 60-65% relative humidity, 12:12 h light: dark cycle, with lights on at 7.30 am). All the animal experiments were carried out according to the internationally valid guidelines following approval by the ‘Zydus Research Center animal ethical committee’.

### Pain Models

### Acetic Acid-Induced Writhing Model

Following oral or i.v. administration of test compounds, mice are rested for 5 min before i.p. injection with 10 ml/kg of 0.6% v/v acetic acid in normal saline. Mice were observed for writhes for 15 min in a 10×10 inch chamber. A writhe is defined as a constriction of the abdominal area, often with extension of the hind legs. Percentage maximum possible effect (MPE) was calculated as below:

% MPE=100−[(No. of writhes in treated mice/No. of writhes in vehicle treated mice)]×100

ED50 dose is determined using GraphPad Prism. Representative data of some of the test compounds are listed in Table-4.

Assessment of CNS Effects of Test Compounds

Test compounds were dissolved in normal saline injected by oral or i.v., routes in ICR mice tail vein. The first dose of 3 mg/kg was injected and mice were observed for spontaneous locomotion and sedation and catalepsy. The dose is scaled down or up if pharmacodynamic effect is present or absent respectively. The lowest dose which shows pharmacodynamic effect was considered threshold dose (TD). Representative data of some of the test compounds are listed in Table-4.

TABLE 4
Invivo ED₅₀ in acetic acid induced pain model and
CNS threshold dose (TD) data of representative compounds

| Compd No. | In-vivo ED₅₀ mg/kg | CNS TD mg/kg |
|---|---|---|
| 6 | 1.1 | 30 |
| 15 | 0.92 | 10 |
| 20 | 1.8 | 30 |
| 22 | 2.5 | 60 |
| 32 | 4.7 | 100 |
| 37 | 6.2 | 60 |
| 43 | 2.1 | 30 |
| 44 | 2.3 | 10 |
| 48 | 3.1 | 30 |
| 57 | 1.9 | 60 |

These compounds are useful in alleviating the pain and suffering inflicted by chronic inflammatory diseases such as rheumatoid arthritis as well as the treatment of gastrointestinal motility disorders such as ileus induced by surgery or peritonitis. A preferred utility is to produce peripheral analgesia without the CNS-mediated side effects of opioids. For example, the abdominal pain induced by laproscopic surgery can be reduced.

The present invention provides a method of treating or preventing a kappa opioid receptor-associated disease or condition in a mammal, such as a human, wherein the method includes administering to the mammal a composition comprising an effective amount of compounds of the general formula (I) of the invention. In another embodiment the kappa opioid receptor-associated conditions are pain, inflammation, pruritis, edema, ileus, tussis or glaucoma.

The novel compounds of the present invention can be formulated into suitable pharmaceutically acceptable compositions by combining with suitable excipients by techniques and processes and concentrations as are well known.

The compounds of formula (I) or pharmaceutical compositions containing them are useful as a medicament as KOR agonist and suitable for humans and other warm blooded animals, and may be administered either by oral, topical or parenteral administration.

Thus, a pharmaceutical composition comprising the compounds of the present invention may comprise a suitable binder, suitable bulking agent &/or diluent and any other suitable agents as may be necessary. Optionally, the pharmaceutical composition may be suitably coated with suitable coating agents.

The compounds of the present invention (I) are KOR agonist and are useful in the treatment or prevention of diseases in which the Kappa (κ) opioid receptors (KOR) are involved, such as treatment or prevention of visceral pain, hyperalgesia, rheumatoid arthritic inflammation, osteoarthritic inflammation, IBD inflammation, IBS inflammation, ocular inflammation, otitic inflammation or autoimmune inflammation.

In one of the embodiments, the present invention of formula (I) can be co-administered in combination with one or more suitable pharmaceutically active agents. In a particular embodiment, the pharmaceutical compositions of the invention can be co-administered with or can include one or more other therapeutic compounds or adjuvants, such as but not limited to other opioids, cannabinoids, antidepressants, anticonvulsants, neuroleptics, antihistamines, acetaminophen, corticosteroids, ion channel blocking agents, non-steroidal anti-inflammatory drugs (NSAIDs) and diuretics, many of which are synergistic in effect with the compounds of the present invention.

Suitable opioids, include, without limitation, alfentanil, alphaprodine, anileridine, bremazocine, codine, dextromoramide, dezocine, diamorphine, dihydrocodeine, dihydromorphine, ethylketazocine, ethylmorphine, fentanyl, hydrocodone, hydromorphone, loperamide, methadone, morphine, nalorphine, oxycodone, oxymorphone, propiram and tramadol.

One embodiment of the invention is co-formulation and/or co-administration of compounds of formula (I) with mu opioid receptor agonist, such as morphine, fentanyl or oxycodone, for the purpose of a mu opioid dose-sparing effect, where the dose of the mu opioid is reduced to minimize common mu opioid side effects, which include constipation, nausea, vomiting, sedation, respiratory depression, itching, mental confusion and seizures.

Suitable antidepressants that can be co-administered with or incorporated into the pharmaceutical compositions of the invention include for example, tricyclic antidepressants such as imipramine, desipramine, trimipramine and clomipramine. Suitable neuroleptics that can be co-administered with or incorporated into the pharmaceutical compositions of the invention include any neuroleptic, for example a compound with D2 dopamine receptor antagonist activity such as domperidone, metoclopramide, zotepine, chlorpromazine, acetophenazine, prochlorperazine and thiothixene. Anticonvulsants such as phenobarbital, phenyloin, carbamazepine, valporic acid, gabapentin and topiramate can also be incorporated into the pharmaceutical compositions of the invention. Muscle relaxants such as methocarbamol, diazepam and chlorzoxazone; anti-migraine agents such as sumitriptan, analeptics sucah as caffeine; antihistamines such as chloropheniramine and pyrilamine; ion channel blocking agents such as sodium ion channel blocker, carbamazepine, calcium ion channel blocker, such as ziconotide; suitable NSAIDs such as aminoarylcarboxylic acid derivatives, arylacetic acid derivatives, arylbutyric acid derivatives, arylpropionic acid derivatives, phenylalkanoic acid derivatives and salicylic acid derivatives, as well as corticosteroids such as methyl-prednisolone, hydrocortisone, cortisone and triameinolone can be incorporated into the pharmaceutical compositions of the present invention.

The quantity of active component, that is, the compounds of Formula (I) according to this invention, in the pharmaceutical composition and unit dosage form thereof may be varied or adjusted widely depending upon the particular application method, the potency of the particular compound and the desired concentration. Generally, the quantity of active component will range between 0.5% to 90% by weight of the composition.

While the present invention has been described in terms of its specific embodiments, certain modifications and equivalents will be apparent to those skilled in the art and are intended to be included within the scope of the present invention.

1. A compound having the structure of general formula (I) 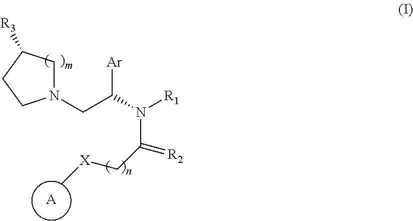
their tautomeric forms, their stereoisomers, their pharmaceutically acceptable salts, and pharmaceutical compositions containing them,
wherein R₁ represents hydrogen, optionally substituted groups selected from C₁₋₆ alkyl, aryl or arylalkyl; R₂═O or NH; R₃ is independently selected from hydroxyl, halogen, hydroxylalkyl, alkoxy, amino, C₁₋₄ alkyl, Aryl, heteroaryl, and cyano; m represents 0, 1 or 2; n represents 0, 1 or 2; X═O or S; ‘Ar’ represents optionally substituted groups selected from aryl, heteroaryl, heterocyclyl, cycloalkylaryl, and cycloalkyl groups; and ‘A’ represents an optionally substituted rings selected from 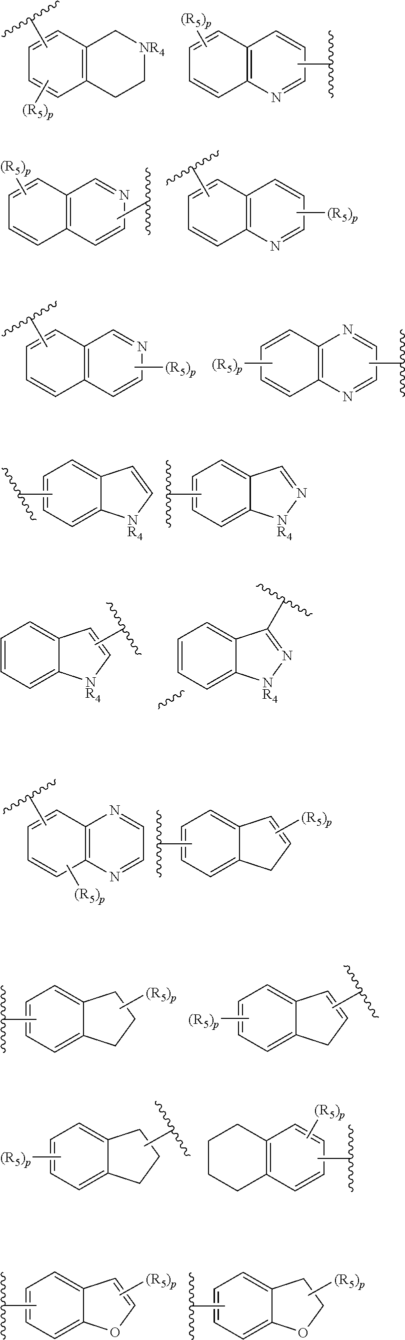 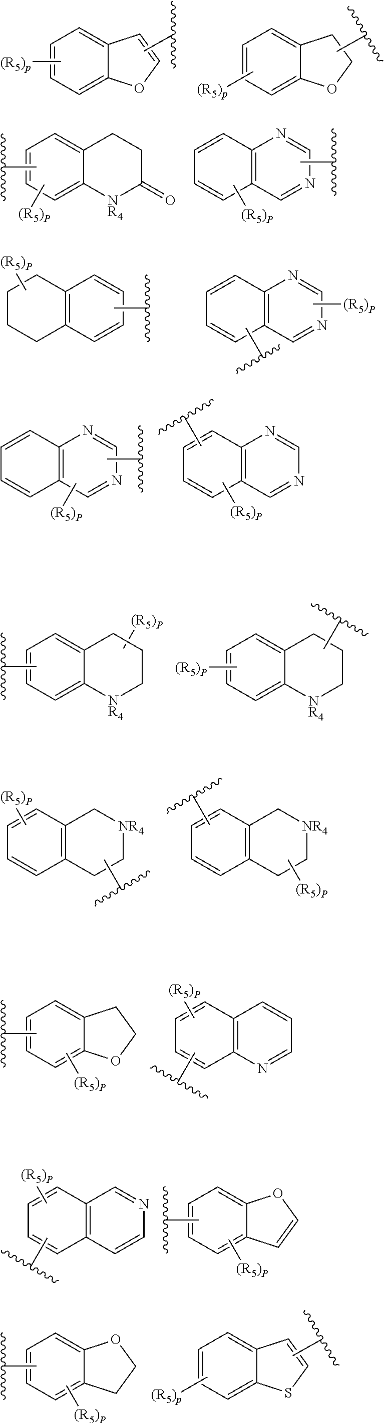 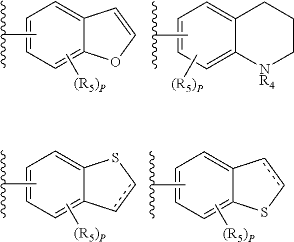
wherein R₄ at each occurrence is independently selected from guanidino, alkyl, haloalkyl, aryl, arylalkyl, heterocyclyl, heteroaryl, —SO₂Rₐ, —SO₂NHRₐ, —COR_{b}, —COOR_{b}, —NHCOOR_{b}; and R₅ at each occurrence is independently selected from cyano, hydroxyl, halogen, guanidino, alkyl, haloalkyl, aryl, arylalkyl, heterocyclyl, heteroaryl, —NHRₐ, —NHSO₂Rₐ, —SO₂Rₐ, —SO₂NHRₐ, —COR_{b}, —COOR_{b}, —NHCOOR_{b}, and —O(CH₂)m-O—(CH₂)m-OH groups, wherein, m=1-8; and Rₐ & R_{b}, in each occurrence is independently selected from hydrogen, alkyl or aryl; and ‘p’ represents an integer from 0-4.

2. The compound as claimed in claim 1 wherein when R₁ is substituted, the substituents on R₁ is independently selected from hydroxy, halo, cyano, amino, (C₁₋₆)alkylamino, C(O)NH(C₁₋₆)alkyl groups;

3. The compound as claimed in claim 1 wherein when Ar is substituted, the substituents on Ar is independently selected from hydroxy, (C₁₋₄)alkoxy, halo, cyano, amino, (C₁₋₆)alkylamino, nitro, COO(C₁₋₄)alkyl, S(O)n, S(O)nNH₂, S(O)nNH(C₁₋₆)alkyl, C(O); C(O)NH(C₁₋₆)alkyl-O(CH₂)m-O—(CH₂)m-OH groups, wherein, n=1-2 and m=1-8.

4. The compound as claimed in claim 1 wherein the heterocyclyl group is selected from tetrahydrofuran (THF), dihydrofuran, 1,4-dioxane, morpholine, 1,4-dithiane, piperazine, piperidine, 1,3-dioxolane, imidazoline, imidazolidine, pyrrolidine, pyrroline, tetrahydropyran, dihydropyran, oxathiolane, dithiolane, 1,3-dioxane, 1,3-dithiane, oxathiane, thiomorpholine.

5. The compound as claimed in claim 1 wherein the heteroaryl group is selected from pyrrolyl, isoxazolyl, isothiazolyl, pyrazolyl, pyridyl, oxazolyl, oxadiazolyl, thiadiazolyl, thiazolyl, imidazolyl, triazolyl, tetrazolyl, furyl, triazinyl, thienyl, pyrimidyl, benzisoxazolyl, benzoxazolyl, benzthiazolyl, benzothiadiazolyl, dihydrobenzofuranyl, indolinyl, pyridazinyl, indazolyl, isoindolyl, dihydrobenzothienyl, indolinyl, pyridazinyl, indazolyl, isoindolyl, dihydrobenzothienyl, indolizinyl, cinnolinyl, phthalazinyl, quinazolinyl, napthyridinyl, carbazolyl, benzodioxolyl, quinoxalinyl, purinyl, furazanyl, isobenzylfuranyl, benzimidazolyl, benzofuranyl, benzothienyl, quinolyl, indolyl, isoquinolyl, dibenzofuranyl.

6. A compound as claimed in claim 1 selected from the group comprising of:
N—((S)-2-((S)-3-hydroxypyrrolidin-1-yl)-1-phenylethyl)-N-methyl-2-(quinolin-2-yloxy)acetamide;
N—((S)-2-((S)-3-hydroxypyrrolidin-1-yl)-1-phenylethyl)-N-methyl-2-((6-methyl sulfonamido)quinolin-2-yloxy)acetamide;
N—((S)-2-((S)-3-hydroxypyrrolidin-1-yl)-1-phenylethyl)-N-methyl-2-((4-methyl sulfonamido)quinolin-2-yloxy)acetamide;
N—((S)-2-((S)-3-hydroxypyrrolidin-1-yl)-1-phenylethyl)-N-methyl-2-((6-methyl sulfonamido)quinolin-4-yloxy)acetamide;
N—((S)-2-((S)-3-hydroxypyrrolidin-1-yl)-1-phenylethyl)-N-methyl-2-((8-methyl sulfonamido)quinolin-5-yloxy)acetamide;
N—((S)-2-((S)-3-hydroxypyrrolidin-1-yl)-1-phenylethyl)-N-methyl-2-((5-methyl sulfonamido)quinolin-8-yloxy)acetamide;
N—((S)-2-((S)-3-hydroxypyrrolidin-1-yl)-1-phenylethyl)-N-methyl-2-((5-nitroquinolin-8-yl)oxy)acetamide;
N—((S)-2-((S)-3-hydroxypyrrolidin-1-yl)-1-phenylethyl)-N-methyl-2-((5-nitroquinolin-1,2,3,4-tetrahydroquinolin-8-yl)oxy)acetamide;
N—((S)-2-((S)-3-hydroxypyrrolidin-1-yl)-1-phenylethyl)-2-(isoquinolin-1-yl oxy)-N-methylacetamide;
N—((S)-2-((S)-3-hydroxypyrrolidin-1-yl)-1-phenylethyl)-N-methyl-2-((4-methyl sulfonamido)isoquinolin-1-yloxy)acetamide;
N—((S)-2-((S)-3-hydroxypyrrolidin-1-yl)-1-phenylethyl)-N-methyl-2-(1,2,3,4-tetra hydroquinolin-8-yloxy)acetamide;
N—((S)-2-((S)-3-hydroxypyrrolidin-1-yl)-1-phenylethyl)-N-methyl-2-((1-(methylsulfonyl)-1,2,3,4-tetrahydroquinolin-8-yl)oxy)acetamide;
N—((S)-2-((S)-3-hydroxypyrrolidin-1-yl)-1-phenylethyl)-N-methyl-2-((1-methyl-1,2,3,4-tetrahydroquinolin-8-yl)oxy)acetamide;
N—((S)-2-((S)-3-hydroxypyrrolidin-1-yl)-1-phenylethyl)-N-methyl-2-((5-(methyl sulfonamido)-1,2,3,4-tetrahydroquinolin-8-yl)oxy)acetamide;
N—((S)-2-((S)-3-hydroxypyrrolidin-1-yl)-1-phenylethyl)-N-methyl-2-((8-(methyl sulfonamido)-1,2,3,4-tetrahydroquinolin-5-yl)oxy)acetamide;
2-((benzofuran-5-yloxy)-N—((S)-2-((S)-3-hydroxy-pyrrolidin-1-yl)-1-phenylethyl)-N-methylacetamide;
2-(benzofuran-6-yloxy)-N—((S)-2-((S)-3-hydroxypyrrolidin-1-yl)-1-phenyl ethyl)-N-methylacetamide;
2-((2,3-dihydrobenzofuran-6-yl)oxy)-N—((S)-2-((S)-3-hydroxypyrrolidin-1-yl)-1-phenylethyl)-N-methylacetamide;
N—((S)-2-((S)-3-hydroxypyrrolidin-1-yl)-1-phenylethyl)-N-methyl-2-(quinolin-6-yloxy)acetamide;
N—((S)-2-((S)-3-hydroxypyrrolidin-1-yl)-1-phenyl ethyl)-N-methyl-2-((1-(methylsulfonyl)-1,2,3,4-tetrahydroquinolin-6-yl)oxy)acetamide;
N—((S)-2-((S)-3-hydroxypyrrolidin-1-yl)-1-phenylethyl)-N-methyl-2-((1,2,3,4-tetrahydroquinolin-6-yl)oxy)acetamide;
N—((S)-2-((S)-3-hydroxy pyrrolidin-1-yl)-1-phenyl ethyl)-N-methyl-2-((1-methyl-8-(methyl sulfonamido)-1,2,3,4-tetrahydroquinolin-5-yl)oxy)acetamide;
N—((S)-2-((S)-3-hydroxypyrrolidin-1-yl)-1-phenylethyl)-N-methyl-2-((8-((N-methyl sulfamoyl)amino)quinolin-5-yl)oxy)-acetamide;
2-((8-((N,N-dimethylsulfamoyl)amino)quinolin-5-yl)oxy)-N—((S)-2-((S)-3-hydroxy pyrrolidin-1-yl)-1-phenyl ethyl)-N-methyl-acetamide;
2-((8-((N,N-diethylsulfamoyl)amino)quinolin-5-yl)oxy)-N—((S)-2-((S)-3-hydroxy pyrrolidin-1-yl)-1-phenyl ethyl)-N-methyl-acetamide;
2-((8-((chloromethylsulfonamido)quinolin-5-yl)oxy)-N—((S)-2-((S)-3-hydroxypyrrolidin-1-yl)-1-phenylethyl)-N-methyl-acetamide;
N—((S)-2-((S)-3-hydroxypyrrolidin-1-yl-)-1-phenylethyl)-N-methyl-2-((8-(methyl sulfonamido)quinoxain-5-yl)oxy)acetamide;
2-((7-bromoquinazolin-2-yl)oxy)-N—((S)-2-((S)-3-hydroxypyrrolidin-1-yl)-1-phenyl ethyl)-N-methyl acetamide;
N—((S)-2-((S)-3-hydroxypyrrolidin-1-yl-)-1-phenylethyl)-2-(indolin-7-yloxy)-N-methyl acetamide;
2-((2,2-dioxido-1,4,5,6-tetrahydro-[1,2,5]thiadiazolo[4,3,2-ij]quinolin-7-yl)oxy-N—((S)-2-((S)-3-hydroxypyrrolidin-1-yl)-1-phenylethyl)-N-methylacetamide;
2-((1-acetyl-8-(methylsulfonamido)-1,2,3,4-tetrahydroquinolin-5-yl)oxy)-N—((S)-2-((S)-3-hydroxypyrrolidin-1-yl)-1-phenylethyl)-N-methylacetamide;
2-((8-((N,N-dimethylsulfamoyl)amino)-1,2,3,4-tetrahydroquinolin-5-yl)oxy)-N—((S)-2-((S)-3-hydroxypyrrolidin-1-yl)-1-phenylethyl)-N-methylacetamide;
2-((8-(2-aminoacetamido)quinolin-5-yl)oxy)-N—((S)-2-((S)-3-hydroxypyrrolidin-1-yl)-1-phenylethyl)-N-methylacetamide;
2-((8-(2-hydroxy acetamido)quinolin-5-yl)oxy)-N—((S)-2-((S)-3-hydroxy pyrrolidin-1-yl)-1-phenylethyl)-N-methylacetamide;
N—((S)-2-((S)-3-hydroxypyrrolidin-1-yl)-1-phenylethyl)-2-((8-(2-methoxy acetamido) quinolin-5-yl)oxy)-N-methylacetamide;
N—((S)-2-((S)-3-hydroxypyrrolidin-1-yl)-1-phenylethyl)-N-methyl-2-((8-sulfonamido) quinolin-5-yloxy)acetamide;
N—((S)-2-((S)-3-hydroxypyrrolidin-1-yl)-1-phenylethyl)-N-methyl-2-((8-sulfonamido)-1,2,3,4-tetrahydroquinolin-5-yloxy)acetamide;
N—((S)-2-((S)-3-hydroxypyrrolidin-1-yl)-1-phenylethyl)-N-methyl-2-((8-(pyrrolidine-1-sulfonamido)quinolin-5-yloxy)acetamide;
N—((S)-2-((S)-3-hydroxypyrrolidin-1-yl)-1-phenylethyl)-N-methyl-2-((8-(pyrrolidine-1-sulfonamido)-1,2,3,4-tetrahydroquinolin-5-yloxy)acetamide;
2-((8-guanidinoquinolin-5-yl)oxy)-N—((S)-2-((S)-3-hydroxypyrrolidin-1-yl)-1-phenyl ethyl)-N-methylacetamide;
2-((8-(2-hydroxy ethyl)amino)quinolin-5-yl)oxy)-N—((S)-2-((S)-3-hydroxypyrrolidin-1-yl)-1-phenylethyl)-N-methylacetamide;
N—((S)-2-((S)-3-hydroxypyrrolidin-1-yl)-1-(3-nitrophenyl)ethyl)-N-methyl-2-((8-(methyl sulfonamido)quinolin-5-yl)oxy) acetamide;
N—((S)-1-(3-aminophenyl)-2-((S)-3-hydroxypyrrolidin-1-yl)ethyl)-N-methyl-2-((8-(methyl sulfonamido)quinolin-5-yl)oxy) acetamide;
N—((S)-2-((S)-3-hydroxypyrrolidin-1-yl)-1-(methylsulfonamido)phenyl)ethyl)-N-methyl-2-((8-(methylsulfonamido)quinolin-5-yl)oxy) acetamide;
N—((S)-1-(3-acetamidophenyl)-2-((S)-3-hydroxypyrrolidin-1-yl)ethyl)-N-methyl-2-((8-(methylsulfonamido)quinolin-5-yl)oxy) acetamide;
N—((S)-2-((S)-3-hydroxypyrrolidin-1-yl)-1-(methylsulfonamido)phenyl)ethyl)-N-methyl-2-(quinolin-5-yl)oxy) acetamide;
N—((S)-1-(3-dimethylaminophenyl)-2-((S)-3-hydroxypyrrolidin-1-yl)ethyl)-N-methyl-2-((8-(methylsulfonamido)quinolin-5-yl)oxy) acetamide;
N—((S)-1-(3-hydroxyphenyl)-2-((S)-3-hydroxypyrrolidin-1-yl)ethyl)-N-methyl-2-((8-(methylsulfonamido)quinolin-5-yl)oxy) acetamide;
N—((S)-2-((S)-3-hydroxypyrrolidin-1-yl)-1-(3-methoxyphenyl)ethyl)-N-methyl-2-((8-(methylsulfonamido)quinolin-5-yl)oxy) acetamide;
Ethyl-2-(3 ((S)-2-((S)-3-hydroxypyrrolidin-1-yl)-1-N-methyl-2-((8-(methylsulfonamido) quinolin-5-yl)oxy)acetamido)ethyl) phenoxy)acetate;
2-(3 ((S)-2-((S)-3-hydroxypyrrolidin-1-yl)-1-N-methyl-2-((8-(methylsulfonamido)quinolin-5-yl)oxy)acetamido)ethyl) phenoxy)acetic acid;
N—((S)-1-(3-fluorophenyl)-2-((S)-3-hydroxypyrrolidin-1-yl)ethyl)-N-methyl-2-((8-(methyl sulfonamido)quinolin-5-yl)oxy) acetamide;
N—((S)-2-((S)-3-hydroxypyrrolidin-1-yl)-1-(3-(trifluoromethyl)phenyl)ethyl)-N-methyl-2-((8-(methylsulfonamido)quinolin-5-yl)oxy) acetamide;
N—((S)-2-((S)-3-hydroxypyrrolidin-1-yl)-1-(3-methyl)ethyl)-N-methyl-2-((8-(methyl sulfonamido)quinolin-5-yl)oxy)acetamide;
2-(benzofuran-6-yloxy)-N—((S)-2-((S)-3-hydroxypyrrolidin-1-yl)-1-(3-nitrophenyl)ethyl)-N-methyl acetamide;
2-(benzofuran-6-yloxy)-N—((S)-2-((S)-3-hydroxypyrrolidin-1-yl)-1-(3-(methyl sulfonamido)phenyl)ethyl)-N-methylacetamide;
2-(benzo[d][1,3]dioxol-5-yloxy)-N—((S)-2-((S)-3-hydroxypyrrolidin-1-yl)-1-(3-(methyl sulfonamido)phenyl)ethyl)-N-methylacetamide;
Methyl-(3-((S)-1-(2-(benzo[d][1,3]dioxol-5-yl oxy)-N-methyl acetamido)-2-((S)-3-hydroxy pyrrolidin-1-yl)-ethyl)phenyl)carbamate;
N—((S)-2-((S)-3-hydroxypyrrolidin-1-yl)-1-phenylethyl)-N-methyl-2-((6-methyl amino)quinolin-2-yloxy)acetamide;
N—((S)-2-((S)-3-hydroxypyrrolidin-1-yl)-1-(4-(methylsulfonamido)phenyl)ethyl)-N-methyl-2-(quinolin-2-yloxy)acetamide;
N—((S)-2-((S)-3-hydroxypyrrolidin-1-yl)-1-phenylethyl)-N-methyl-2-((4-methyl amino) quinolin-2-yloxy)acetamide;
N—((S)-2-((S)-3-hydroxypyrrolidin-1-yl)-1-phenylethyl)-N-methyl-2-(quinolin-4-yl oxy) acetamide;
N—((S)-2-((S)-3-hydroxypyrrolidin-1-yl)-1-phenylethyl)-N-methyl-2-((8-methylamino) quinolin-5-yloxy)acetamide;
N—((S)-2-((S)-3-hydroxypyrrolidin-1-yl)-1-(4-(methylsulfonamido)phenyl)ethyl)-N-methyl-2-(quinolin-5-yloxy)acetamide;
N—((S)-2-((S)-3-hydroxypyrrolidin-1-yl)-1-phenylethyl)-N-methyl-2-((5-methyl amino) quinolin-8-yloxy)acetamide;
N—((S)-2-((S)-3-hydroxypyrrolidin-1-yl)-1-(4-(methylsulfonamido)phenyl)ethyl)-N-methyl-2-(quinolin-8-yloxy)acetamide;
N—((S)-2-((S)-3-hydroxypyrrolidin-1-yl)-1-phenylethyl)-N-methyl-2-((4-methyl amino) isoquinolin-1-yloxy)acetamide;
N—((S)-2-((S)-3-hydroxypyrrolidin-1-yl)-1-(4-(methylsulfonamido)phenyl)ethyl)2-(isoquinolin-1-yloxy)-N-methylacetamide;
N—((S)-2-((S)-3-hydroxypyrrolidin-1-yl)-1-phenylethyl)-N-methyl-2-((6-methyl amino) isoquinolin-1-yloxy)acetamide;
N—((S)-2-((S)-3-hydroxypyrrolidin-1-yl)-1-phenylethyl)-N-methyl-2-(quinazolin-2-yloxy)acetamide;
N—((S)-2-((S)-3-hydroxypyrrolidin-1-yl)-1-phenylethyl)-N-methyl-2-((6-methyl amino) quinazolin-2-yloxy)acetamide;
N—((S)-2-((S)-3-hydroxypyrrolidin-1-yl)-(4-(methylsulfonamido)phenyl)ethyl)-N-methyl-2-(1,2,3,4-tetrahydroquinolin-8-yloxy)acetamide;
N—((S)-2-((S)-3-hydroxypyrrolidin-1-yl)-(4-(methylsulfonamido)phenyl)ethyl)-N-methyl-2-(1,2,3,4-tetrahydroquinolin-5-yloxy)acetamide;
2-((benzofuran-5-yloxy)-N—((S)-2-((S)-3-hydroxy-pyrrolidin-1-yl)-1-(4-(methyl sulfonamido)phenyl)ethyl)-N-methylacetamide;
2-((2,3-dihydrobenzofuran-5-yloxy)-N—((S)-2-((S)-3-hydroxy-pyrrolidin-1-yl)-1-(4-(methylsulfonamido)phenyl)ethyl)-N-methyl acetamide;
N—((S)-2-((S)-3-hydroxypyrrolidin-1-yl)-1-(naphthalene-1-yl)ethyl)-N-methyl-2-((5-((methylsulfonamido)-quinolin-8-yl)oxy)acetamide;
N—((S)-2-((S)-3-hydroxypyrrolidin-1-yl)-1-(naphthalene-1-yl)ethyl)-N-methyl-2-((5-((methyl amino)-quinolin-8-yl)oxy)acetamide;
N—((S)-2-((S)-3-hydroxypyrrolidin-1-yl)-1-(naphthalene-1-yl)ethyl)-N-methyl-2-((8-((methylsulfonamido)-quinolin-5-yl)oxy)acetamide;
N—((S)-2-((S)-3-hydroxypyrrolidin-1-yl)-1-(naphthalene-1-yl)ethyl)-N-methyl-2-((8-((methyl amino)-quinolin-5-yl)oxy)acetamide;
N—((S)-2-((S)-3-hydroxy pyrrolidin-1-yl)-1-(naphthalene-1-yl)ethyl)-N-methyl-2-((1,2,3,4-tetrahydroquinolin-8-yl)oxy)acetamide;
N—((S)-2-((S)-3-hydroxypyrrolidin-1-yl)-1-(naphthalene-1-yl)ethyl)-N-methyl-2-((1-(methylsulfonyl)-1,2,3,4-tetrahydroquinolin-8-yl)oxy)acetamide;
2-(benzofuran-5-yloxy)-N—((S)-2-((S)-3-hydroxypyrrolidin-1-yl)-1-(naphthalene-1-yl)ethyl)-N-methylacetamide;
2-((2,3-dihydrobenzofuran-5-yloxy)-N—((S)-2-((S)-3-hydroxy pyrrolidin-1-yl)-1-(naphthalene-1-yl)ethyl)-N-methyl acetamide;
N—((S)-2-((S)-3-hydroxypyrrolidin-1-yl)-1-phenylethyl)-N-methyl-2-((1,2,3,4-tetrahydroquinolin-4-yl)oxy)acetamide;
N—((S)-2-((S)-3-hydroxypyrrolidin-1-yl)-1-phenyl ethyl)-N-methyl-2-((1-(methylsulfonyl)-1,2,3,4-tetrahydroquinolin-4-yl)oxy)acetamide;
2-((2,3-dihydrobenzo[b]thiophen-6-yl)oxy)-N—((S)-2-((S)-3-hydroxypyrrolidin-1-yl)-1-phenylethyl)-N-methylacetamide;
2-(benzo[b]thiophen-6-yloxy)-N—((S)-2-((S)-3-hydroxypyrrolidin-1-yl)-1-phenyl ethyl)-N-methylacetamide;
2-((2,3-dihydrobenzo[b]thiophen-5-yl)oxy)-N—((S)-2-((S)-3-hydroxypyrrolidin-1-yl)-1-phenylethyl)-N-methylacetamide;
2-((8-(2-hydroxy ethoxy)ethyl)amino)quinolin-5-yl)oxy)-N—((S)-2-((S)-3-hydroxypyrrolidin-1-yl)-1-phenylethyl)-N-methylacetamide;
2-((8-(1,3-dihydroxypropan-2-yl)amino)quinolin-5-yl)oxy)-N—((S)-2-((S)-3-hydroxypyrrolidin-1-yl)-1-phenylethyl)-N-methylacetamide;
N—((S)-1-((3-(2-(2-hydroxy ethoxy)ethoxyl)phenyl)-2-((S)-3-hydroxypyrrolidin-1-yl)ethyl)-N-methyl-2-((8-(methylsulfonamido)quinolin-5-yl)oxy)acetamide;
N—((S)-1-((3-((1,3-dihydroxypropan-2-yl)oxy)phenyl)-2-((S)-3-hydroxypyrrolidin-1-yl)ethyl)-N-methyl-2-((8-(methylsulfonamido)quinolin-5-yl)oxy)acetamide;
N—((S)-2-((S)-3-hydroxypyrrolidin-1-yl)-1-phenylethyl)-N-methyl-2-((8-(methylsulfonamido)-1-(methylsulfonyl)-1,2,3,4-tetrahydroquinolin-5-yl)oxy)acetamide;
N—((S)-2-((S)-3-hydroxypyrrolidin-1-yl)-1-phenylethyl)-N-methyl-2-((5-(methylsulfonamido)-1-(methylsulfonyl)-1,2,3,4-tetrahydroquinolin-8-yl)oxy)acetamide;
N—((S)-2-((S)-3-hydroxypyrrolidin-1-yl)-1-phenylethyl)-N-methyl-2-((8-(piperidine-1-sulfonamido)quinolin-5-yl)oxy)acetamide;
N—((S)-2-((S)-3-hydroxypyrrolidin-1-yl)-1-phenylethyl)-N-methyl-2-((8-(piperidine-1-sulfonamido)-1,2,3,4-tetrahydroquinolin-5-yl)oxy)acetamide;
N—((S)-2-((S)-3-hydroxypyrrolidin-1-yl)-1-phenylethyl)-N-methyl-2-((8-(morpholine-4-sulfonamido)quinolin-5-yl)oxy)acetamide;
N—((S)-2-((S)-3-hydroxypyrrolidin-1-yl)-1-phenylethyl)-N-methyl-2-((8-(morpholine-4-sulfonamido)-1,2,3,4-tetrahydroquinolin-5-yl)oxy)acetamide;
N—((S)-2-((S)-3-hydroxypyrrolidin-1-yl)-1-phenylethyl)-N-methyl-2-((8-(phenylsulfonamido))quinolin-5-yl)oxy)acetamide;
N—((S)-2-((S)-3-hydroxypyrrolidin-1-yl)-1-phenylethyl)-N-methyl-2-((8-(phenylsufonamido)-1,2,3,4-tetrahydroquinolin-5-yl)oxy)acetamide;
N—((S)-2-((S)-3-hydroxypyrrolidin-1-yl)-1-phenylethyl)-N-methyl-2-(quinolin-2-ylthio)acetamide;
N—((S)-2-((S)-3-hydroxy pyrrolidin-1-yl)-1-phenylethyl)-N-methyl-2-(quinolin-8-ylthio)acetamide;
N—((S)-2-((S)-3-hydroxypyrrolidin-1-yl)-1-phenylethyl)-N-methyl-2-((8-methylsulfonamido)quinolin-5-ylthio)acetamide;
N—((S)-2-((S)-3-hydroxypyrrolidin-1-yl)-1-(methylsulfonamido)phenyl)ethyl)-N-methyl-2-(quinolin-5-yl)thio) acetamide;
N—((S)-2-((S)-3-hydroxypyrrolidin-1-yl)-1-phenylethyl)-N-methyl-2-(1,2,3,4-tetrahydroquinolin-8-ylthio)acetamide;
N—((S)-2-((S)-3-hydroxypyrrolidin-1-yl)-1-(pyridin-4-yl)ethyl)-N-methyl-2-((1,2,3,4-tetrahydroquinolin-8-yl)oxy)acetamide;
N—((S)-2-((S)-3-hydroxypyrrolidin-1-yl)-1-(pyridin-3-yl)ethyl)-N-methyl-2-((1,2,3,4-tetrahydroquinolin-8-yl)oxy)acetamide;
N—((R)-2-((S)-3-hydroxy pyrrolidin-1-yl)-1-(pyridin-2-yl)ethyl)-N-methyl-2-((1,2,3,4-tetrahydroquinolin-8-yl)oxy)acetamide

7. The compound as claimed in claim 1 selected from the group comprising of:
N—((S)-2-((S)-3-hydroxypyrrolidin-1-yl)-1-phenylethyl)-N-methyl-2-(quinolin-2-yloxy) acetamide;
N—((S)-2-((S)-3-hydroxy pyrrolidin-1-yl)-1-phenylethyl)-N-methyl-2-((6-methyl sulfonamido)quinolin-2-yloxy)acetamide;
N—((S)-2-((S)-3-hydroxy pyrrolidin-1-yl)-1-phenylethyl)-N-methyl-2-((4-methyl sulfonamido)quinolin-2-yloxy)acetamide;
N—((S)-2-((S)-3-hydroxy pyrrolidin-1-yl)-1-phenylethyl)-N-methyl-2-((6-methyl sulfonamido)quinolin-4-yloxy)acetamide;
N—((S)-2-((S)-3-hydroxy pyrrolidin-1-yl)-1-phenylethyl)-N-methyl-2-((8-methyl sulfonamido)quinolin-5-yloxy)acetamide;
N—((S)-2-((S)-3-hydroxy pyrrolidin-1-yl)-1-phenylethyl)-N-methyl-2-((5-methyl sulfonamido)quinolin-8-yloxy)acetamide;
N—((S)-2-((S)-3-hydroxy pyrrolidin-1-yl)-1-phenylethyl)-N-methyl-2-((5-nitroquinolin-8-yl)oxy)acetamide;
N—((S)-2-((S)-3-hydroxypyrrolidin-1-yl)-1-phenylethyl)-N-methyl-2-((5-nitro quinolin-1,2,3,4-tetrahydroquinolin-8-yl)oxy)acetamide;
N—((S)-2-((S)-3-hydroxypyrrolidin-1-yl)-1-phenylethyl)-2-(isoquinolin-1-yl oxy)-N-methyl acetamide;
N—((S)-2-((S)-3-hydroxypyrrolidin-1-yl)-1-phenylethyl)-N-methyl-2-((4-methyl sulfonamido)isoquinolin-1-yloxy)acetamide;
N—((S)-2-((S)-3-hydroxypyrrolidin-1-yl)-1-phenylethyl)-N-methyl-2-(1,2,3,4-tetra hydroquinolin-8-yloxy)acetamide;
N—((S)-2-((S)-3-hydroxypyrrolidin-1-yl)-1-phenyl ethyl)-N-methyl-2-((1-(methylsulfonyl)-1,2,3,4-tetrahydroquinolin-8-yl)oxy)acetamide;
N—((S)-2-((S)-3-hydroxypyrrolidin-1-yl)-1-phenylethyl)-N-methyl-2-((1-methyl-1,2,3,4-tetrahydroquinolin-8-yl)oxy)acetamide;
N—((S)-2-((S)-3-hydroxypyrrolidin-1-yl)-1-phenylethyl)-N-methyl-2-((5-(methyl sulfonamido)-1,2,3,4-tetrahydroquinolin-8-yl)oxy)acetamide;
N—((S)-2-((S)-3-hydroxypyrrolidin-1-yl)-1-phenylethyl)-N-methyl-2-((8-(methyl sulfonamido)-1,2,3,4-tetrahydroquinolin-5-yl)oxy)acetamide;
2-((benzofuran-5-yloxy)-N—((S)-2-((S)-3-hydroxy-pyrrolidin-1-yl)-1-phenylethyl)-N-methylacetamide;
2-(benzofuran-6-yloxy)-N—((S)-2-((S)-3-hydroxypyrrolidin-1-yl)-1-phenyl ethyl)-N-methyl acetamide;
2-((2,3-dihydrobenzofuran-6-yl)oxy)-N—((S)-2-((S)-3-hydroxypyrrolidin-1-yl)-1-phenylethyl)-N-methylacetamide;
N—((S)-2-((S)-3-hydroxypyrrolidin-1-yl)-1-phenylethyl)-N-methyl-2-(quinolin-6-yl oxy) acetamide;
N—((S)-2-((S)-3-hydroxypyrrolidin-1-yl)-1-phenyl ethyl)-N-methyl-2-((1-(methylsulfonyl)-1,2,3,4-tetrahydroquinolin-6-yl)oxy)acetamide;
N—((S)-2-((S)-3-hydroxypyrrolidin-1-yl)-1-phenylethyl)-N-methyl-2-((1,2,3,4-tetrahydro quinolin-6-yl)oxy)acetamide;
N—((S)-2-((S)-3-hydroxy pyrrolidin-1-yl)-1-phenyl ethyl)-N-methyl-2-((1-methyl-8-(methyl sulfonamido)-1,2,3,4-tetrahydroquinolin-5-yl)oxy)acetamide;
N—((S)-2-((S)-3-hydroxypyrrolidin-1-yl)-1-phenylethyl)-N-methyl-2-((8-((N-methyl sulfamoyl)amino)quinolin-5-yl)oxy)-acetamide;
2-((8-((N,N-dimethylsulfamoyl)amino)quinolin-5-yl)oxy)-N—((S)-2-((S)-3-hydroxy pyrrolidin-1-yl)-1-phenyl ethyl)-N-methyl-acetamide;
2-((8-((N,N-diethylsulfamoyl)amino)quinolin-5-yl)oxy)-N—((S)-2-((S)-3-hydroxy pyrrolidin-1-yl)-1-phenyl ethyl)-N-methyl-acetamide;
2-((8-((chloromethylsulfonamido)quinolin-5-yl)oxy)-N—((S)-2-((S)-3-hydroxypyrrolidin-1-yl)-1-phenylethyl)-N-methyl-acetamide;
N—((S)-2-((S)-3-hydroxypyrrolidin-1-yl-)-1-phenylethyl)-N-methyl-2-((8-(methyl sulfonamido)quinoxain-5-yl)oxy)acetamide;
2-((7-bromoquinazolin-2-yl)oxy)-N—((S)-2-((S)-3-hydroxypyrrolidin-1-yl)-1-phenyl ethyl)-N-methyl acetamide;
N—((S)-2-((S)-3-hydroxypyrrolidin-1-yl-)-1-phenylethyl)-2-(indolin-7-yloxy)-N-methyl acetamide;
2-((2,2-dioxido-1,4,5,6-tetrahydro-[1,2,5]thiadiazolo[4,3,2-ij]quinolin-7-yl)oxy-N—((S)-2-((S)-3-hydroxypyrrolidin-1-yl)-1-phenylethyl)-N-methylacetamide;
2-((1-acetyl-8-(methylsulfonamido)-1,2,3,4-tetrahydroquinolin-5-yl)oxy)-N—((S)-2-((S)-3-hydroxypyrrolidin-1-yl)-1-phenylethyl)-N-methylacetamide;
2-((8-((N,N-dimethylsulfamoyl)amino)-1,2,3,4-tetrahydroquinolin-5-yl)oxy)-N—((S)-2-((S)-3-hydroxypyrrolidin-1-yl)-1-phenylethyl)-N-methylacetamide;
2-((8-(2-aminoacetamido)quinolin-5-yl)oxy)-N—((S)-2-((S)-3-hydroxypyrrolidin-1-yl)-1-phenylethyl)-N-methylacetamide;
2-((8-(2-hydroxy acetamido)quinolin-5-yl)oxy)-N—((S)-2-((S)-3-hydroxy pyrrolidin-1-yl)-1-phenylethyl)-N-methylacetamide;
N—((S)-2-((S)-3-hydroxypyrrolidin-1-yl)-1-phenylethyl)-2-((8-(2-methoxy acetamido) quinolin-5-yl)oxy)-N-methylacetamide;
N—((S)-2-((S)-3-hydroxypyrrolidin-1-yl)-1-phenylethyl)-N-methyl-2-((8-sulfonamido) quinolin-5-yloxy)acetamide;
N—((S)-2-((S)-3-hydroxypyrrolidin-1-yl)-1-phenylethyl)-N-methyl-2-((8-sulfonamido)-1,2,3,4-tetrahydroquinolin-5-yloxy)acetamide;
N—((S)-2-((S)-3-hydroxypyrrolidin-1-yl)-1-phenylethyl)-N-methyl-2-((8-(pyrrolidine-1-sulfonamido)quinolin-5-yloxy)acetamide;
N—((S)-2-((S)-3-hydroxypyrrolidin-1-yl)-1-phenylethyl)-N-methyl-2-((8-(pyrrolidine-1-sulfonamido)-1,2,3,4-tetrahydroquinolin-5-yloxy)acetamide;
2-((8-guanidinoquinolin-5-yl)oxy)-N—((S)-2-((S)-3-hydroxypyrrolidin-1-yl)-1-phenyl ethyl)-N-methylacetamide;
2-((8-(2-hydroxy ethyl)amino)quinolin-5-yl)oxy)-N—((S)-2-((S)-3-hydroxypyrrolidin-1-yl)-1-phenylethyl)-N-methylacetamide;
N—((S)-2-((S)-3-hydroxypyrrolidin-1-yl)-1-(3-nitrophenyl)ethyl)-N-methyl-2-((8-(methyl sulfonamido)quinolin-5-yl)oxy) acetamide;
N—((S)-1-(3-aminophenyl)-2-((S)-3-hydroxypyrrolidin-1-yl)ethyl)-N-methyl-2-((8-(methyl sulfonamido)quinolin-5-yl)oxy) acetamide;
N—((S)-2-((S)-3-hydroxypyrrolidin-1-yl)-1-(methylsulfonamido)phenyl)ethyl)-N-methyl-2-((8-(methylsulfonamido)quinolin-5-yl)oxy) acetamide;
N—((S)-1-(3-acetamidophenyl)-2-((S)-3-hydroxypyrrolidin-1-yl)ethyl)-N-methyl-2-((8-(methylsulfonamido)quinolin-5-yl)oxy) acetamide;
N—((S)-2-((S)-3-hydroxypyrrolidin-1-yl)-1-(methylsulfonamido)phenyl)ethyl)-N-methyl-2-(quinolin-5-yl)oxy) acetamide;
N—((S)-1-(3-dimethyl aminophenyl)-2-((S)-3-hydroxypyrrolidin-1-yl)ethyl)-N-methyl-2-((8-(methylsulfonamido)quinolin-5-yl)oxy) acetamide;
N—((S)-1-(3-hydroxyphenyl)-2-((S)-3-hydroxypyrrolidin-1-yl)ethyl)-N-methyl-2-((8-(methylsulfonamido)quinolin-5-yl)oxy) acetamide;
N—((S)-2-((S)-3-hydroxypyrrolidin-1-yl)-1-(3-methoxyphenyl)ethyl)-N-methyl-2-((8-(methylsulfonamido)quinolin-5-yl)oxy) acetamide;
Ethyl-2-(3 ((S)-2-((S)-3-hydroxypyrrolidin-1-yl)-1-N-methyl-2-((8-(methylsulfonamido) quinolin-5-yl)oxy)acetamido)ethyl) phenoxy)acetate;
2-(3 ((S)-2-((S)-3-hydroxypyrrolidin-1-yl)-1-N-methyl-2-((8-(methylsulfonamido)quinolin-5-yl)oxy)acetamido)ethyl) phenoxy)acetic acid;
N—((S)-1-(3-fluorophenyl)-2-((S)-3-hydroxypyrrolidin-1-yl)ethyl)-N-methyl-2-((8-(methyl sulfonamido)quinolin-5-yl)oxy) acetamide;
N—((S)-2-((S)-3-hydroxypyrrolidin-1-yl)-1-(3-(trifluoromethyl)phenyl)ethyl)-N-methyl-2-((8-(methylsulfonamido)quinolin-5-yl)oxy) acetamide;
N—((S)-2-((S)-3-hydroxypyrrolidin-1-yl)-1-(3-methyl)ethyl)-N-methyl-2-((8-(methylsulfonamido)quinolin-5-yl)oxy)acetamide;
2-(benzofuran-6-yloxy)-N—((S)-2-((S)-3-hydroxypyrrolidin-1-yl)-1-(3-nitrophenyl)ethyl)-N-methyl acetamide;
2-(benzofuran-6-yloxy)-N—((S)-2-((S)-3-hydroxypyrrolidin-1-yl)-1-(3-(methylsulfonamido)phenyl)ethyl)-N-methylacetamide;
2-(benzo[d][1,3]dioxol-5-yloxy)-N—((S)-2-((S)-3-hydroxypyrrolidin-1-yl)-1-(3-(methyl sulfonamido)phenyl)ethyl)-N-methylacetamide;
Methyl-(3-((S)-1-(2-(benzo[d][1,3]dioxol-5-yloxy)-N-methylacetamido)-2-((S)-3-hydroxypyrrolidin-1-yl)-ethyl)phenyl)carbamate;

8. A pharmaceutical composition comprising a therapeutically effective amount of a compound of Formula (I) as claimed in claim 1 and optionally one or more pharmaceutically acceptable carriers, diluents or excipients.

9. A method of treating or preventing a kappa opioid receptor-associated disease which comprising administering to a patient in need thereof an effective amount of a compound of formula (I) as claimed in claim 1 or its suitable pharmaceutical composition.

10. A compound of formula (I) or their pharmaceutical composition useful as a medicament as KOR agonist and suitable for humans and other warm blooded animals.

11. Use of a compound as claimed in claim 1 in the preparation of a medicament for the treatment or prevention of visceral pain, hyperalgesia, rheumatoid arthritic inflammation, osteoarthritic inflammation, IBD inflammation, IBS inflammation, ocular inflammation, otitic inflammation or autoimmune inflammation.

12. A pharmaceutical composition comprising the compounds of formula (I) as claimed in claim 1 in combination with one or more pharmaceutically active agents selected from group comprising opioids, cannabinoids, antidepressants, anticonvulsants, neuroleptics, antihistamines, acetaminophen, corticosteroids, ion channel blocking agents, non-steroidal anti-inflammatory drugs (NSAIDs) and diuretics or pharmaceutically acceptable salts thereof.

13. The use of the pharmaceutical composition as claimed in claim 12 for the treatment of visceral pain, hyperalgesia, rheumatoid arthritic inflammation, osteoarthritic inflammation, IBD inflammation, IBS inflammation, ocular inflammation, otitic inflammation or autoimmune inflammation.
